SEQUENCE LISTING (31 sequences)

SEQ ID NO: 1 - RNA, 20 nt, synthetic construct
  source 1..20: mol_type = other RNA
ggcatgtccgaattgccatc

SEQ ID NO: 2 - DNA, 127 bp, synthetic construct
  source 1..127: mol_type = other DNA
tccccccaaagctctccctgcaagtgacagaactgagctgaaatagacatcaggcacacttgctggcacactcaagcagctccattcgaatggcaatacagacatgccagcctagagggatcagtcg

SEQ ID NO: 3 - DNA, 23 bp, synthetic construct
  source 1..23: mol_type = other DNA; note = Forward PCR primer for Rs1emR209C mice
gactaggcttcctctttctcttt

SEQ ID NO: 4 - DNA, 21 bp, synthetic construct
  source 1..21: mol_type = other DNA; note = Reverse PCR primer for Rs1emR209C mice
agatatagccccattcatccc

SEQ ID NO: 5 - DNA, 23 bp, synthetic construct
  source 1..23: mol_type = other DNA; note = Reverse primer for screeing Rs1emR209C mice. The primer pairs used for screening tmutation site of Rs1 gene
aaatccttattggcattgaatcc

SEQ ID NO: 6 - DNA, 21 bp, synthetic construct
  source 1..21: mol_type = other DNA; note = Forward primer for sXBP1
ctgagtccgaatcaggtgcag

SEQ ID NO: 7 - DNA, 21 bp, synthetic construct
  source 1..21: mol_type = other DNA; note = Reverse primer for sXBP1
gtccatgggaagatgttctgg

SEQ ID NO: 8 - DNA, 20 bp, synthetic construct
  source 1..20: mol_type = other DNA; note = Forward primer for ATF4
gtggccaagcacttgaaacc

SEQ ID NO: 9 - DNA, 20 bp, synthetic construct
  source 1..20: mol_type = other DNA; note = Reverse primer for ATF4
ggaaaaggcatcctccttgc

SEQ ID NO: 10 - DNA, 24 bp, synthetic construct
  source 1..24: mating_type = Forward primer for HSPA5; mol_type = other DNA
ttcagccaattatcagcaaactct

SEQ ID NO: 11 - DNA, 26 bp, synthetic construct
  source 1..26: mol_type = other DNA; note = Reverse primer for HSPA5
ttttctgatgtatcctcttcaccagt

SEQ ID NO: 12 - DNA, 22 bp, synthetic construct
  source 1..22: mol_type = other DNA; note = Forward primer for DDIT3
catacaccaccacacctgaaag

SEQ ID NO: 13 - DNA, 22 bp, synthetic construct
  source 1..22: mol_type = other DNA; note = Reverse primer for DDIT3
ccgtttcctagttcttccttgc

SEQ ID NO: 14 - DNA, 22 bp, synthetic construct
  source 1..22: mol_type = other DNA; note = Forward primer for DNAJC3
acgcctttgacggtgccgatta

SEQ ID NO: 15 - DNA, 22 bp, synthetic construct
  source 1..22: mol_type = other DNA; note = Forward primer for GRP94
aagtcgctgatggctttcctgg

SEQ ID NO: 16 - DNA, 20 bp, synthetic construct
  source 1..20: mol_type = other DNA; note = Forward primer for GRP94
gaccttcgggttcgtcagag

SEQ ID NO: 17 - DNA, 20 bp, synthetic construct
  source 1..20: mol_type = other DNA; note = Reverse primer for GRP94
agccttctcggcttttaccc

SEQ ID NO: 18 - DNA, 20 bp, synthetic construct
  source 1..20: mol_type = other DNA; note = Forwad primer for beta-actin
ttgctgacaggatgcagaag

SEQ ID NO: 19 - DNA, 20 bp, synthetic construct
  source 1..20: mol_type = other DNA; note = Reverse primer for beta-actin
gtacttgcgctcaggaggag

SEQ ID NO: 20 - DNA, 23 bp, synthetic construct
  source 1..23: mol_type = other DNA; note = Forward primer for LAMP1
cagcactctttgaggtgaaaaac

SEQ ID NO: 21 - DNA, 21 bp, synthetic construct
  source 1..21: mol_type = other DNA; note = Reverse primer for LAMP1
acgatctgagaaccattcgca

SEQ ID NO: 22 - DNA, 22 bp, synthetic construct
  source 1..22: mol_type = other DNA; note = Forward primer for LAMP2
atatgtgcaacaaagagcaggt

SEQ ID NO: 23 - DNA, 23 bp, synthetic construct
  source 1..23: mol_type = other DNA; note = Reverse primer for LAMP2
tgccaattaggtaagcaatcact

SEQ ID NO: 24 - DNA, 19 bp, synthetic construct
  source 1..19: mol_type = other DNA; note = Forward primer for SOD1
cagaaggcaagcggtgaac

SEQ ID NO: 25 - DNA, 23 bp, synthetic construct
  source 1..23: mol_type = other DNA; note = Reverse primer for SOD1
cagccttgtgtattgtccccata

SEQ ID NO: 26 - DNA, 20 bp, synthetic construct
  source 1..20: mol_type = other DNA; note = Forward primer for Catalse
ggacgctcagcttttcattc

SEQ ID NO: 27 - DNA, 20 bp, synthetic construct
  source 1..20: mol_type = other DNA; note = Reverse primer for Catalse
ggacgctcagcttttcattc

SEQ ID NO: 28 - DNA, 20 bp, synthetic construct
  source 1..20: mol_type = other DNA; note = Forward primer for Caspase 3
tgcagcatgctgaagctgta

SEQ ID NO: 29 - DNA, 20 bp, synthetic construct
  source 1..20: mol_type = other DNA; note = Reverse primer for Caspase 3
gagcatggacacaatacacg

SEQ ID NO: 30 - DNA, 21 bp, synthetic construct
  source 1..21: mol_type = other DNA; note = Forward primer for Fas
gctgcagacatgctgtggatc

SEQ ID NO: 31 - DNA, 21 bp, synthetic construct
  source 1..21: mol_type = other DNA; note = Reverse primer for Fas
tcacagccaggagaatcgcag

(19) United States
(12) Patent Application Publication
(10) Pub. No.: US 2026/0115323 A1
(43) Pub. Date: Apr. 30, 2026

(54) METHOD FOR TREATING X-LINKED RETINOSCHISIS

(71) Applicant: Taipei Veterans General Hospital, Taipei City (TW)

(72) Inventors: Shih-Hwa CHIOU, Taipei City (TW); Shih-Jen CHEN, Taipei City (TW); De-Kuang HWANG, Taipei City (TW); Yueh CHIEN, Taipei City (TW); Yi-Ping YANG, Taipei City (TW); Shih-Jie CHOU, Taipei City (TW); Tai-Chi LIN, Taipei City (TW)

(73) Assignee: Taipei Veterans General Hospital, Taipei City (TW)

(21) Appl. No.: 19/222,731

(22) Filed: May 29, 2025

Related U.S. Application Data
(60) Provisional application No. 63/706,489, filed on Oct. 11, 2024.

(51) Int. Cl. A61K 48/00; A61K 38/17; A61P 27/02; C12Q 1/6883; G01N 33/50

(52) U.S. Cl. CPC A61K 48/0058; A61K 38/1732; A61P 27/02; C12Q 1/6883; G01N 33/5091; C12Q 2600/156

(57) ABSTRACT

The present invention provides a multiomics approach, which integrate single-cell RNA-sequencing (scRNA-seq) and spatiotemporal transcriptomics (ST) offering potential for dissecting transcriptional networks and revealing cell-cell interactions involved in biomolecular pathomechanisms. The present invention also provides a multimodal approach combining high-throughput scRNA-seq and ST to elucidate XLRS-specific transcriptomic signatures in two XLRS-like models with retinal splitting phenotypes, including genetically engineered (Rs1emR209C) mice and patient-derived retinal organoids harboring the same patient-specific p.R209C mutation. Through multiomics transcriptomic analysis, the endoplasmic reticulum (ER) stress/eIF2 signaling, mTOR pathway, and the regulation of eIF4 and p70S6K pathways as chronically enriched and highly conserved disease pathways between two XLRS-like models are identified. Western blots and proteomics analysis validated the occurrence of unfolded protein responses, chronic eIF2α signaling activation, and chronic ER stress-induced apoptosis. Furthermore, therapeutic targeting of the chronic ER stress/eIF2α pathway activation synergistically enhanced the efficacy of AAV mediated RS1 gene delivery, ultimately improving bipolar cell integrity, postsynaptic transmission, disorganized retinal architecture and electrophysiological responses. Collectively, the complex transcriptomic signatures obtained from Rs1emR209C mice and patient-derived retinal organoids using the multiomics approach provide opportunities to unravel potential therapeutic targets for incurable retinal diseases, such as XLRS.

### RELATED APPLICATION

This non-provisional application claims the benefit under 35 U.S.C. § 119(e) to U.S. Provisional Application No. 63/706,489, filed on Oct. 11, 2024, which is hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 8, 2026, is named “CSH0026US-sequence listing.xml” and is 34,056 bytes in size. The sequence listing contained in this .XML file is part of the specification and is hereby incorporated by reference herein in its entirety.

### FIELD OF THE INVENTION

The present invention relates to a new method for treating a retinal disease, such as X-linked retinoschisis (X-linked juvenile retinoschisis; XLRS).

### BACKGROUND OF THE INVENTION

X-linked retinoschisis (X-linked juvenile retinoschisis; XLRS) is an early-onset X-linked inherited retinal disorder that leads to structural splitting of the neurosensory retina and severe visual impairment [1]. Retinoschisin-1 (RS1) is a 224-amino acid protein consisting of two conserved regions: an N-terminal signal sequence (aa 1-23) and a discoidin domain (aa 64-219). Secreted by retinal cells, RS1 forms a homo-octameric complex through disulfide bonds, maintaining retinal structural integrity by binding to photoreceptors and bipolar cells [2]. The pathogenic mechanisms of XLRS have been extensively investigated by numerous elegant studies using Rs1-knockout mice which perfectly recapitulated XLRS phenotypes [2b, 3]. Although AAV based RS1 gene delivery has indeed shown promising efficacy in rescuing retinoschisis in Rs1-knockout mice [2b, 4], AAV-based RS1 gene therapy unexpectedly yielded unsatisfactory outcomes after an 18-month treatment in a clinical trial [5]. However, the exact factors contributed to the ineffective treatment of RS1 gene therapy in XLRS patients remain unknown. To further optimize treatment strategies for XLRS, it is critically important to elucidate the precise pathogenic transcriptomic mechanisms.

Disease modeling is crucial for recapitulating phenotypes, studying pathogenesis, and conducting drug screening, thereby enhancing data reliability and clinical translation. In XLRS, over 190 mutations in the Retinoschisin-1 (RS1) gene, primarily located within the discoidin domain [6], have been identified and are widely recognized as the primary cause of XLRS pathogenesis [7]. Despite the valuable insights gained from XLRS research using Rs1-knockout mice [2b, 3-4], this model does not incorporate the specific clinically relevant mutations found in patients. Mutations in the RS1 gene can interfere with its octamer structure formation, leading to the production of misfolded RS1 protein. This misfolding impedes the protein's transport to the cell surface, significantly contributing to the pathologies associated with XLRS [8]. In this context, some research groups have developed knock-in models with patient-specific Rs1 mutations, providing crucial insights into how specific mutations contribute to pathological phenotypes in XLRS [9]. For example, Chen et al. developed a RS1-knock-in mice harboring the causative mutation (RS1, p. Y65X) and these mice exhibited decreased b-wave amplitude in the electroretinogram with the splitting of inner nuclear layer (INL), shortened inner segments and reduced outer segment numbers [9a]. Liu et al. further generated knock-in mice with different patient-specific point mutations, i.e., C59S and R141C point mutants, and demonstrated that the early-onset XLRS-like disease phenotype is genotype-dependent [9b]. Regarding in vitro platforms modeling XLRS, patient-induced pluripotent stem cell (iPSC)-derived organoids also harbor patient-specific mutations and hold promising potential for personalized therapy [10]. Utilizing their 3D structure and functions, these organoids bridge the gap between in vitro studies and clinical applications [11]. Patient iPSC-derived organoids have been used to model neurodegeneration diseases [12] and cystic fibrosis [13]. Previously, we generated retinal organoids from XLRS patient iPSCs, and these patient-derived retinal organoids replicate patient specific mutations and splitting phenotypes [10a]. These observations highlighted the importance of the distinct contribution of specific point mutations to disease severity in XLRS.

Modern omics technologies have shown promising potential in identifying disease biomarkers and screening potential therapeutics, therefore exploiting their merits in biomedical or translational applications [14]. Single-cell RNA sequencing (scRNA-seq) is a recently rising technology and powerful tool for investigating cellular heterogeneity and measuring gene expression at an individual cell level [15]. The technique has been extensively used for biomarker. identification [14], pathogenesis investigation [16], and disease diagnosis [17]. Remarkably, scRNA-seq has also been utilized in human retinas and retina organoids to examine the changes in transcriptomic landscape during eye development [18] and the alterations of biological pathways in ocular diseases, including age-related macular degeneration, autoimmune retinopathy, and retinoblastoma [19]. More recently, the spatial transcriptomics (ST) technique has been developed utilizing in situ experiments to understand the gene expression and cell-cell interaction spatially. Relying on such technologies, new subtypes of retinal cells have been identified based on their spatial arrangement [20]. ScRNA-seq and ST have been combined to decipher the molecular mechanisms during retinal development [21]. However, these integrative transcriptomic approaches have not yet been utilized in retinal diseases.

Accordingly, it is still desirable to develop a new method for treating a retinal disease, such as X-linked retinoschisis (XLRS).

### BRIEF SUMMARY OF THE INVENTION

Accordingly, the present invention provides a multimodal sequencing platform that integrates scRNA-seq and ST may allow the spatial assessment of the differential gene expression between individual cells within the interested niche at single-cell levels, further providing us the opportunities for exploring disease pathogenesis of incurable vision disorders, such as XLRS.

In one aspect, the present invention provides a multiomics approach, which integrate single-cell RNA-sequencing (scRNA-seq) and spatiotemporal transcriptomics (ST) offering potential for dissecting transcriptional networks and revealing cell-cell interactions involved in biomolecular pathomechanisms.

In another aspect, the present invention provides a multimodal system, comprising high-throughput scRNA-seq and ST to elucidate XLRS-specific transcriptomic signatures.

The present invention also provides two disease models: a genetically engineered mice Rs1emR209C, and a patient-derived retinal organoids harboring the patient-specific p.R209C mutation.

In the invention, the disease model is a X-linked retinoschisis (XLRS)-like model.

According to the invention, the endoplasmic reticulum (ER) stress/eIF2 signaling, mTOR pathway, and the regulation of eIF4 and p70S6K pathways are identified as chronically enriched and highly conserved disease pathways.

In a further aspect, the present invention provides a method for treating retinal disease in a subject, which comprising administering to the subject an AAV mediated RS1 gene delivery together with a therapeutic agent targeting the endoplasmic reticulum (ER) stress/eIF2 signaling, mTOR pathway, and the regulation of eIF4 and p70S6K pathways, which ultimately improves bipolar cell integrity, postsynaptic transmission, disorganized retinal architecture and electrophysiological responses.

In one example of the present invention, the retinal disease is X-linked retinoschisis (XLRS).

In the present invention, a multiomic approach integrating ST and scRNA-seq was proposed to investigate the intricate transcriptomic signatures associated with XLRS. CRISPR-Cas9 technology was utilized to create genetically engineered mice carrying the patient-specific p.R209 mutation (Rs1emR209C). These Rs1emR209C mice exhibited typical XLRS-like phenotypes, including retinal splitting and impaired electrophysiological function. Using the integrated multiomic approach, we analyzed the transcriptomic signatures of the Rs1emR209C mouse retina as well as the XLRS patient derived retinal organoids, as described in our previous work [10a]. By identifying overlapping transcriptomic signatures between these two models, we pinpointed the most enriched disease related pathways and delineated potential pathogenic mechanisms. Through subsequent wet-lab experiments and liquid chromatography-mass spectrometry (LC-MS)-based proteomic analysis, we validated the chronic activation and enrichment of identified disease-related pathways. Notably, therapeutic targeting of the chronic activation of ER stress/eIF2 signaling using salubrinal, a small molecule specific inhibitor that suppresses chronic ER stress-induced apoptosis [22] and alleviates ER workload [23], mitigated XLRS-like phenotypes in Rs1emR209C retinas. Combining this targeted therapy with AAV-based RS1 gene delivery demonstrated synergistic efficacy, suggesting a novel and effective treatment strategy for the currently incurable XLRS.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows that a multimodal approach integrating high-throughput scRNA-seq and ST to elucidate XLRS-specific transcriptomic signatures in genetically engineered (Rs1emR209C) mice and patient-specific iPSC-derived 3D-retinal organoids (iPSC-3DRO) harboring the patient-specific R209C mutation; wherein the most enriched disease pathway was identified and targeted therapeutically, showing synergistic efficacy when combined with AAV-based Rs1 delivery in ameliorating XLRS phenotypes.

FIGS. 2A-2I shows the characterizing XLRS patient-derived retinal organoids and genetically engineered Rs1 knock-in mice carrying patient-specific point mutation p. R209C. FIG. 2A provides the schematic illustration showing the disease modeling using XLRS patient-derived retinal organoids and genetically engineered Rs1 knock-in mice (Rs1emR209C mice). Note that the patient-derived retinal organoids and Rs1emR209C mice carried the point mutation p. R209C identical to that of the enrolled XLRS patient. FIG. 2B shows the results of clinical examination of XLRS patient with p. R209C point mutation by OCT imaging and color fundus photography. FIG. 2C provides the design of the CRISPR/Cas9 genome editing system to generate Rs1emR209C mice by introducing sgRNA to target the PAM sequence on the wild-type allele. FIG. 2D provides the confirmation of introducing point mutation p. R209C into Rs1 in Rs1emR209C mice using Sanger sequencing. FIG. 2E shows the OCT imaging and FIG. 2(F) shows the H & E staining of Rs1emR209C and age matched wild-type mouse retinas at different ages. FIG. 2G shows the IF staining shows the expression of bipolar cell marker, PRKCA (green), and postsynaptic marker, DLG4 (red) in Rs1emR209C and age-matched wild-type mouse retinas at different ages. Nuclei were stained with DAPI (blue). FIG. 2H provides IF staining and quantification of GFAP stained müller glia (green) and in Rs1emR209C and wild-type retinas at different ages. FIG. 2(I) Dark-adapted ERG responses in Rs1emR209C and age-matched wild-type mouse retinas at different ages. Scale bar=60 □m.

FIGS. 3A-3N show the generation of XLRS patient-specific iPSC differentiated retinal organoids and Rs1emR209C mice. FIG. 3A shows the phenotypic determination of normal and XLRS patient-specific iPSC-differentiated retinal organoids at postinduction 150 days under bright field microscopy and H & E staining. FIG. 3B The brightfield images of healthy control and XLRS patient-derived retinal organoids at 90 days, 120 days, and 150 days postinduction. FIG. 3C shows the Sanger Sequencing result of human iPSC control (top) and XLRS human patient iPSC (bottom). FIG. 3D shows the confirmation of RS1 point mutation site of XLRS patient-specific iPSCs using Sanger sequencing. FIG. 3E provides a graph showing PCR targets and primer sequences to determine wild-type and knock-in allele. FIG. 3F provides a scheme of the breeding setup to generate Rs1emR209C mice colony. FIG. 3G shows the result of Sanger Sequencing showed nucleotide changes in the founder Rs1emR209C mice. FIG. 3H shows the Sanger Sequencing of wild-type and Rs1emR209C mice. FIG. 3I provides the representative images and FIG. 3J provides the quantification of DAPI stained photoreceptor cell density in the ONL from Rs1emR209C and wild-type retinas at different ages. FIG. 3K provides the quantification of GRAP positive areas from Rs1emR209C and wild-type retinas at different ages, including FIG. 3L showing the scotopic awave, FIG. 3M showing scotopic b-wave, and FIG. 3N showing the a-wave implicit time of Rs1emR209C and age matched wild-type mouse retinas at different ages.

FIGS. 4A-4K show the ScRNAseq analysis of Rs1emR209C mice and XLRS patient iPSC-derived retinal organoids. FIG. 4A provides a schematic illustration showing the multiomic approach integrating scRNA-seq and ST to identify overlapping disease pathways on patient-derived retinal organoids and Rs1emR209C mice. The transcriptomic signatures were identified by deep scRNA-seq analysis from both models. The overlapping transcriptomic signatures identified by scRNA-seq delineated the crucial disease-related pathways. ST was used for validating the disease-related pathways identified by scRNA-seq. FIG. 4B provides the diagram showing the scRNA-seq experimental procedures of XLRS patient-derived retinal organoids and Rs1emR209C mice. FIG. 4C shows the IF staining of RS1 (green) and bipolar cell marker PKCA (red) in Rs1emR209C and wild-type retinas at different ages. Nuclei were stained with DAPI (blue). FIG. 4D provides the UMAP visualization and cell clustering of Rs1emR209C and age matched wild-type mouse retinas. FIG. 4E shows the relative proportions of cell clusters in Rs1emR209C and wildtype retinas at different ages. FIG. 4F shows the up-regulated and down-regulated DEGs of scRNA-seq dataset in bipolar cells from Rs1emR209C and wild-type mouse retinas at different ages. FIG. 4G provides the IPA results showing the enriched pathways in the bipolar cells, cones, and rods of the Rs1emR209C retinas at indicated times. FIG. 4H provides the Venn diagrams show the number of overlapped pathways enriched in bipolar cells, cones, and rods between Rs1emR209C retinas and XLRS patient-specific retinal organoids. FIG. 4I shows the overlapped pathways enriched in bipolar cells, cones, and rods of Rs1emR209C retinas and XLRS patient-specific retinal organoids. FIG. 4J provides the Venn diagram showing the number of overlapped enriched pathways across bipolar cells, cones, and rods in Rs1emR209C retinas and XLRS patient-specific retinal organoids. FIG. 4K provides the gene interaction analysis revealing the interaction network among the genes involved in eIF2 signaling, mTOR and the regulation of the eIF4 and p70S6K signaling pathways.

FIGS. 5A-5F shows the DEG identification and functional analysis of human retinal organoids and mouse retinas. The Left of FIG. 5A provides the heat map of average expression of each gene in each cell type in mouse retinas. The Right of FIG. 5A provides the heat map of average expression of each gene in each cell type in human retinal organoids. The Upper of FIG. 5B provides a separate view of the expression of cell-type specific marker genes in each cell type in mouse retinas. The Lower of FIG. 5B provides a separate view of the expression of cell-type specific marker genes in each cell type in human retinal organoids. FIG. 5C shows the clustering of different cell types based on cell type-specific markers in XLRS patient-specific RS/R209C/y mutation and control iPSC differentiated retinal organoids. FIG. 5D shows the up-regulated and down-regulated DEGs of scRNA-seq dataset in bipolar cells from XLRS patient iPSC- and ctrl iPSC-derived retinal organoids at different post-induction days. FIG. 5E Upper provides the volcano plot showing the DEGs in cone and bipolar cells of 3-week-old, 6-month-old and 12-month-old mouse retinas. FIG. 5E Lower provides the volcano plot showing the DEGs in rod, cone and bipolar cells of human retinal organoids. FIG. 5F shows the enrichment of eIF2 signaling and eIF2-related signaling pathways in human retinal organoids (Upper) and mouse retinas (Lower).

FIGS. 6A-6H show the Chronic enrichment of ER stress/eIF2α pathway in RS1-expressing cells in Rs1emR209C mouse retinas. FIG. 6A provides the Xenium analysis showing the visualization of selected marker genes to identify retinal ganglion cells, rod and cone photoreceptors, retinal pigment epithelium, microglia, and endothelial cells. The original H&E-stained section is presented in the upper left corner. FIG. 6B provides the Xenium analysis showing the enrichment of eIF2α pathway, FIG. 6C provides the regulation of eIF4 and p70S6K signaling pathway, and FIG. 6D provides the mTOR signaling pathway in Rs1emR209C retinas compared to age-matched wild-type retinas. Transcripts per area were quantified and shown as the lower subpanel in FIGS. 6B-6D, i.e., panels B-D (N=3, mean±s.e.). FIG. 6E provides the using of CytAssist Visium to match the gene expression captured spots in the spatial transcriptome (right) in the H&E-stained images of 3 week-old wild-type and Rs1emR209C retinas (left). The captured spots were further divided into different retinal layers, including the retinal ganglion cell layer (blue), inner cell layer (orange), outer cell layer (green), and retinal pigment epithelium (red). FIG. 6F provides the heatmap showing the expression of CytAssist Visium-detected enriched genes in bulk RNAseq and the rod cell data from scRNAseq. CytAssist Visium-captured spots shows the gene expression of (Panel G; Upper, FIG. 6G) Edn2 and (FIG. 6H, Panel H; Upper) Gnb3 in wild-type and RsemR209C retinas. scRNA-seq and UMAP visualization show the distribution of Fdn2 and Gnb3 expression in cell clusters (FIG. 6G-6H, Panels G, H; Lower).

FIGS. 7A-7E show that Visualization of retinal cell types using Xenium analysis. FIG. 7A provides Xenium analysis visualizing selected marker genes associated with specific retinal cell types. FIG. 7B provides the identification of specific retinal cell types using selected marker genes in mouse retinas. FIG. 7C shows the analysis of the eIF2 signaling pathway after completing a layered analysis of the retinal cells from Rs1emR209C retinas and wild-type retinas. FIG. 7D provides the functional analysis of overlapping enriched genes in RNAseq, scRNAseq and spatial transcriptome. FIG. 7E provides the heatmap showing the expression of CytAssist Visium-detected enriched genes in bulk RNAseq and the rod, cone, and bipolar cell data from scRNAseq. CytAssist Visium-captured spots showing the gene expression of Fdn2 and Gnb3 in wild-type and Rs1emR209C retinas.

FIG. 8 shows the comparison of selected gene expression in Rs1emR209C and wild-type mouse retinas. qRT-PCR results of the gene expression associated with the ER stress pathway and other disease-related pathways in Rs1emR209C and wild-type mouse retinas.

FIGS. 9A-9Q show the misfolding of RS1 protein activates the ER stress/eIF2 pathway in Rs1emR209C mouse retinas. FIG. 9A provides the nonreducing SDS polyacrylamide gradient gels showing the failure of RS1 homo-octameric complex in Rs1emR209C mouse retinas (upper). In reducing gels, a large reduction of RS1 protein was observed in the lysates from Rs1emR209C mouse retinas (lower). FIG. 9B provides the quantification of RS1 protein of wild-type and Rs1emR209C mouse retinas in the reducing gel. Western blot shows increased (1) BIP protein amount (FIG. 9C), (2) the phosphorylation of eIF2α (FIG. 9D), (3) ATF4 and CHOP protein amount in Rs1emR209C retinas compared to wild-type retinas (FIG. 9E). FIG. 9F provides the quantification of BIP protein amount, FIG. 9G provides the quantification of eIF2α phosphorylation, FIG. 9(H) provides the quantification of ATF4, and FIG. 9I provides the quantification of CHOP protein amount (N=3, mean±s.e.). FIG. 9J shows the Puromycin incorporation assay indicating failure of protein synthesis in Rs1emR209C retinas (N=2, mean±s.e.). FIG. 9K provides the quantification of protein synthesis through puromycin incorporation assay. FIG. 9L provides the TUNEL assay showing increased apoptosis in the Rs1emR209C retinas.

FIG. 9M provides the IF staining showing increased phosphorylation of PERK in Rs1emR209C retinas at 6 months and 12 months, but no PERK phosphorylation was detected in wild-type retinas at any given age. FIG. 9N provides the Mass spectrometry (LC/MS)-based proteomic analysis showing the reduction in overall translation in the Rs1emR209C retinas (N=4). FIG. 9O provides the Comparison of the score sequest HT: sequest HT between wild-type and Rs1emR209C retinas at 3-week ages (N=3, mean±s.e.). One-way t-test, *p-value <0.05. FIG. 9P provide the heatmap comparing the expression of the candidate proteins involved in XLRS pathologies between wild-type and Rs1emR209C retinas at 3-week ages. FIG. 9Q provides the schematic illustration shows RS1 protein misfolding chronically enriched ER stress and eIF2α pathway, impaired protein synthesis, leading to increased ER stress-induced apoptosis in Rs1emR209C retinas.

FIG. 10 shows the schematic procedures for salubrinal treatment. Experimental procedures showing the phenotypic examination of salubrinal-treated Rs1emR209C mice using OCT imaging, H&E staining, and electroretinogram.

FIGS. 11A-11P show the therapeutic targeting the chronic ER stress/eIF2 pathway activation ameliorates retinoschisis and improves retinal electrophysiological functions. FIG. 11A provides the OCT imaging of Rs1emR209C retinas with or without salubrinal treatment. FIG. 11B provides the quantification of the schisis area based on the OCT imaging data of Rs1emR209C retinas with or without salubrinal treatment (N=10, mean±s.e.). FIG. 11C provides the quantification of the ONL thickness based on the OCT imaging of Rs1emR209C retinas with or without salubrinal treatment (N=10, mean±s.e.). FIG. 11D shows the H & E staining of Rs1emR209C retinas with or without salubrinal treatment. FIG. 11E provides the quantification of the schisis area based on the H&E staining of Rs1emR209C retinas with or without salubrinal treatment (N=10, mean±s.e.). FIG. 11F provides the quantification of the ONL thickness based on the H&E staining data of Rs1emR209C retinas with or without salubrinal treatment (N=10, mean±s.e.). FIG. 11G shows the intensity-response relation for the dark adapted ERG of Rs1emR209C retinas with or without salubrinal treatment. FIG. 11H shows the scotopic a-waves, FIG. 11I shows scotopic b-waves, and FIG. 11J shows the a-wave implicit time of Rs1emR209C retinas with and without salubrinal treatment. In panels H, I, and J, the results are mean±s.e. of eight independent experiments. FIG. 11K shows IF staining of bipolar cell marker PKCA (red), FIG. 11L shows postsynaptic marker DLG4 (yellow), and FIG. 11M shows photoreceptor marker recoverin (purple) in Rs1emR209C retinas treated with salubrinal or PBS. Nuclei were stained with DAPI (blue). FIG. 11(N) shows the quantification of the PKCA-positive cells, and intensity of the area positive for DLG4 (FIG. 11O), and recoverin in Rs1emR209C retinas treated with salubrinal or PBS (FIG. 11P). In FIGS. 11N, 11O and 11P, i.e., panels N, O, and P, the results are mean±s.e. of three independent experiments.

FIGS. 12A-12L show the in situ spatial transcriptomics of differential gene expression in Rs1emR209C mouse retinas. FIG. 12A shows the using of the Xenium ST to visualize several cell types in Rs1emR209C mouse retina receiving the administration of salubrinal or PBS. Xenium analysis and quantification of the transcripts per area of the Xenium data showing the treatment effect of salubrinal on the enrichment of eIF2α pathway (FIG. 12B), regulation of eIF4 and p70S6K signaling pathway (FIG. 12C), and mTOR signaling pathway in Rs1emR209C retinas compared to PBS treated retinas (FIG. 12(D)) (N=3, mean±s.e.). Xenium analysis and IF show the enrichment of Gnb3 (FIG. 12(E)) and Edn2 at transcriptomic (left) and protein levels (middle and right) in Rs1emR209C retinas compared to age-matched wildtype retinas (FIG. 12F). Xenium analysis and IF show the effect of salubrinal treatment on the enrichment of Gnb3 (FIG. 12G) and Edn2 gene at transcriptomic (left) and protein levels (middle and right) in Rs1emR209C retinas compared to PBS-treated retina (FIG. 12H). The quantification of (Gnb3 (FIG. 12I) and Edn2 (FIG. 12J) expression based on the Xenium analysis and IF data for Rs1emR209C retinas and wild-type retinas (N=3, mean±s.e.). The quantification of Gnb3 (FIG. 12K) and Edn2 (FIG. 12L) expression based on the Xenium analysis and IF data for Rs1emR209C retinas treated with salubrinal or PBS (N=3, mean±s.e.).

FIGS. 13A-13M show the combination of salubrinal administration and AAV-based RS1 gene delivery synergistically ameliorates retinoschisis and improves retinal electrophysiological functions in Rs1emR209C mouse retinas. FIG. 13A provides the experimental design showing the combination of salubrinal administration and AAV-based RS1 gene delivery in Rs1emR209C retinas. FIG. 13B provides the CHOP staining and FIG. 13C provides the TUNEL assay in PBS-treated Rs1emR209C retinas, AAV8-based RS1 gene delivery, and the combination of salubrinal and AAV8-based RS1 delivery. FIG. 13D provides OCT imaging of Rs1emR209C PBS treated retinas, AAV8-based RS1 gene delivery, and the combination of salubrinal and AAV8-based RS1 delivery. The schisis splitting area (pink) is presented in the lower right corner. FIG. 13E provides the quantification of CHOP protein content (N=4, mean±s.e.), FIG. 13F provides the quantification of apoptotic signals (N=4, mean±s.e.), FIG. 13G provides the area of schisis splitting cavities (N=7, mean±s.e.), and FIG. 13H shows ONL thickness (N=8, mean±s.e.) in Rs1emR209C retinas with indicated treatment. FIG. 13I shows the intensity-response relation for the dark-adapted full-field ERG of PBS-treated Rs1emR209C mouse retinas, AAV8-based RS1 gene delivery, and the combination of salubrinal and AAV8-based RS1 delivery. FIG. 13J provides the comparison of the scotopic a-wave amplitudes, FIG. 13(K) provides the comparison of the scotopic b-wave amplitudes, and FIG. 13L provides the comparison of the a-wave implicit time of the PBS-treated Rs1emR209C retinas, AAV8-based RS1 gene delivery, and the combination of salubrinal and AAV8-based RS1 delivery (N=8, mean±s.e.). FIG. 13M provides a schematic diagram shows the enrichment of ER stress/eIF2 signaling pathways in XLRS pathogenesis. Misfolded RS1 protein increased ER stress, BIP, phosphorylated eIF2, ATF4, and CHOP. This signaling cascade leads to the increase of apoptosis of retinal cells. Salubrinal that ameliorates ER stress and attenuates the eIF2 pathway exhibited a synergistic efficacy with AAV-based RS1 gene delivery in the treatment of XLRS.

FIG. 14 shows the schematic illustration showing the use of multiomic approaches to identify XLRS related disease pathways in in vitro and in vivo XLRS-like models. For disease modeling (Upper), patient's peripheral blood was collected for the generation of patient iPSCs and patient iPSC-derived retinal organoids. These patient-derived retinal organoids carried patient's genetic background and unique splitting features. Meanwhile, using a CRISPR-Cas9-based technologies, we also generated the genetically engineered mice (Rs1emR209C mice) which exhibited XLRS-like retinoschisis phenotypes. Sanger sequencing confirmed that both XLRS-like models harbored the same patient-specific Rs1 point mutation. For the multiomic approach (Middle), we subjected the dissociated single cells from both patient-derived retinal organoids and Rs1emR209C mouse retina to scRNA-seq. The transcriptomic signatures of both XLRS-like models identified by scRNA-seq were integrated and overlapped to obtain the highly enriched and conserved pathways between two XLRS-like models. In situ spatial transcriptomics was used to validate the enrichment of identified disease-related pathways. The signal transduction of the disease-related pathways were verified using immunofluorescence and Western blot. For the therapeutic targeting of the most enriched disease-related pathway, a pharmacological agent was administered to target the most enriched disease pathway in the Rs1emR209C mice. The efficacy of therapeutic targeting on the enrichment of disease pathways were verified using in situ spatial transcriptomics. Its efficacy on disease phenotypes were examined using OCT and ERG. Therapeutic targeting plus AAV8-mediated Rs1 gene delivery further showed synergistic efficacy in Rs1emR209C mice. The therapeutic targeting inactivates the disease pathways and the gene therapy provided new wildtype RS1 protein.

### DETAILED DESCRIPTION OF THE INVENTION

Unless defined otherwise, all technical and scientific terms used herein have the same meaning as commonly understood by a person skilled in the art to which this invention belongs.

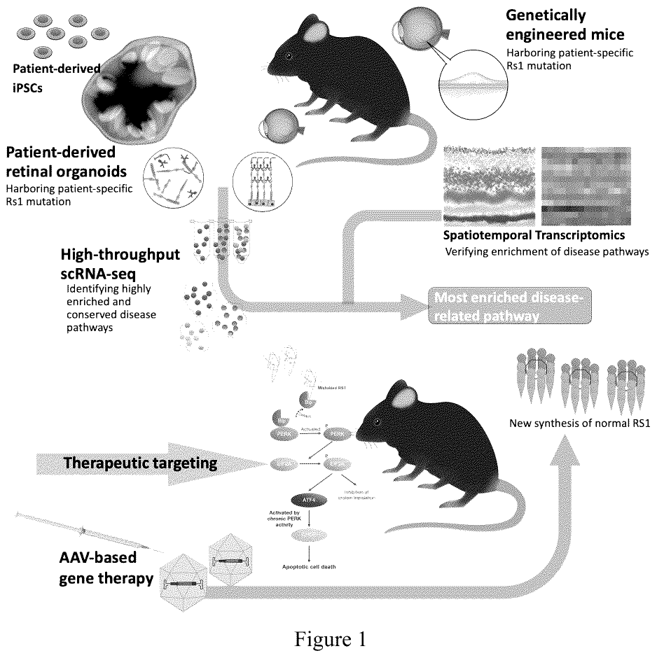

The present invention provides a new method for treating X-linked retinoschisis (XLRS). As demonstrated in FIG. 1, a multimodal approach integrating high-throughput scRNA-seq and ST to elucidate XLRS-specific transcriptomic signatures in genetically engineered (Rs1emR209C) mice and patient-specific iPSC-derived 3D-retinal organoids (iPSC-3DRO) harboring the patient-specific R209C mutation. The most enriched disease pathway was identified and targeted therapeutically, showing synergistic efficacy when combined with AAV-based Rs1 delivery in ameliorating XLRS phenotypes.

X-linked retinoschisis (XLRS) is an inherited retinal disorder with severe retinoschisis and visual impairments. Multiomics approaches integrate single-cell RNA-sequencing (scRNA-seq) and spatiotemporal transcriptomics (ST) offering potential for dissecting transcriptional networks and revealing cell-cell interactions involved in biomolecular pathomechanisms. Herein, we demonstrated a multimodal approach combining high-throughput scRNA-seq and ST to elucidate XLRS-specific transcriptomic signatures in two XLRS-like models with retinal splitting phenotypes, including genetically engineered (Rs1emR209C) mice and patient-derived retinal organoids harboring the same patient-specific p.R209C mutation. Through multiomics transcriptomic analysis, we identified the endoplasmic reticulum (ER) stress/eIF2 signaling, mTOR pathway, and the regulation of eIF4 and p70S6K pathways as chronically enriched and highly conserved disease pathways between two XLRS-like models. Western blots and proteomics analysis validated the occurrence of unfolded protein responses, chronic eIF2α signaling activation, and chronic ER stress-induced apoptosis. Furthermore, therapeutic targeting of the chronic ER stress/eIF2α pathway activation synergistically enhanced the efficacy of AAV mediated RS1 gene delivery, ultimately improving bipolar cell integrity, postsynaptic transmission, disorganized retinal architecture and electrophysiological responses. Collectively, the complex transcriptomic signatures obtained from RsemR209C mice and patient-derived retinal organoids using the multiomics approach provide opportunities to unravel potential therapeutic targets for incurable retinal diseases, such as XLRS.

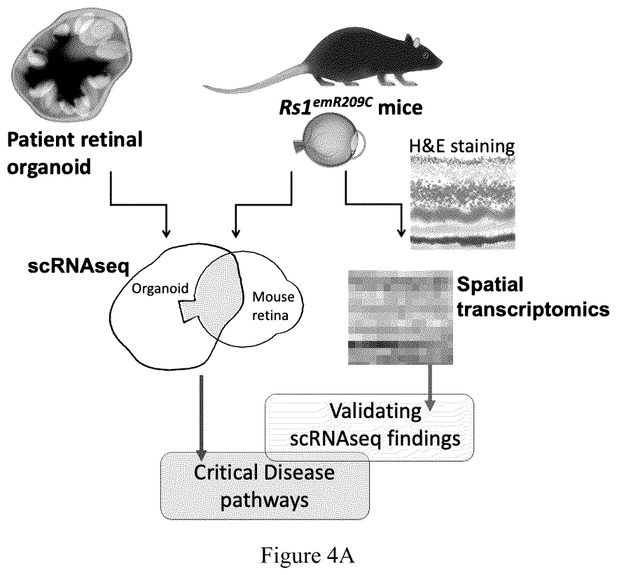

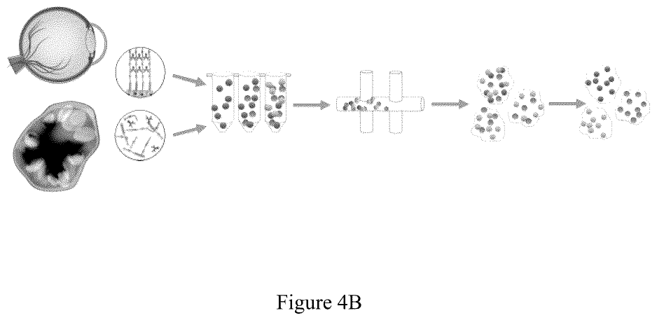

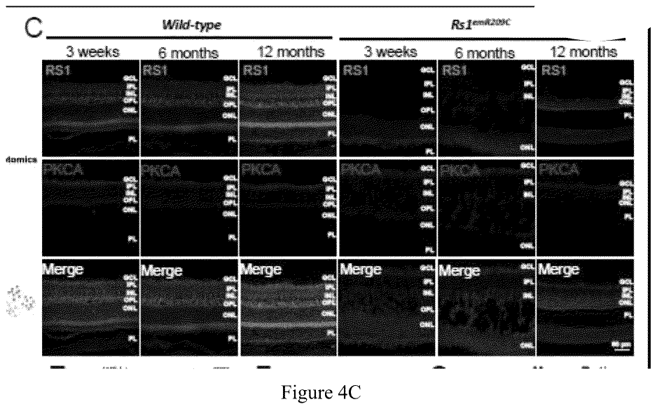

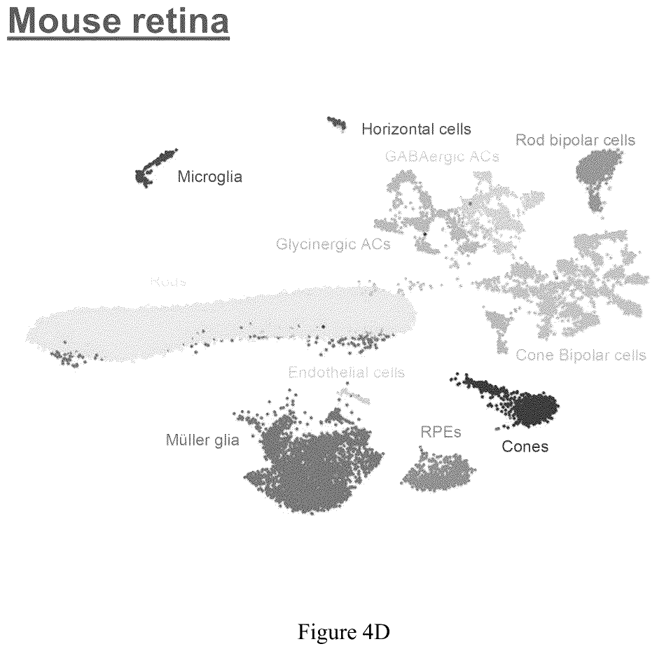

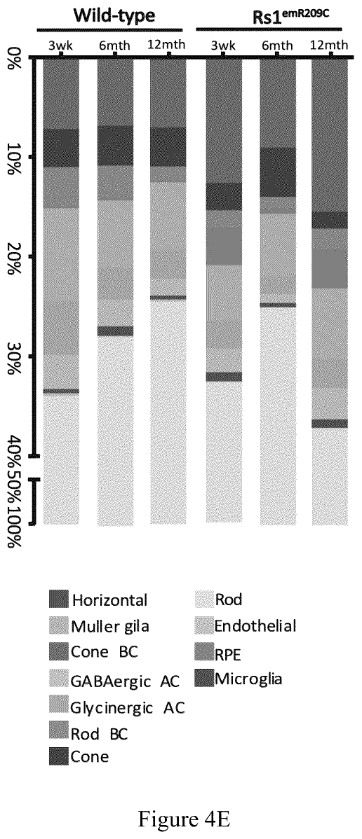

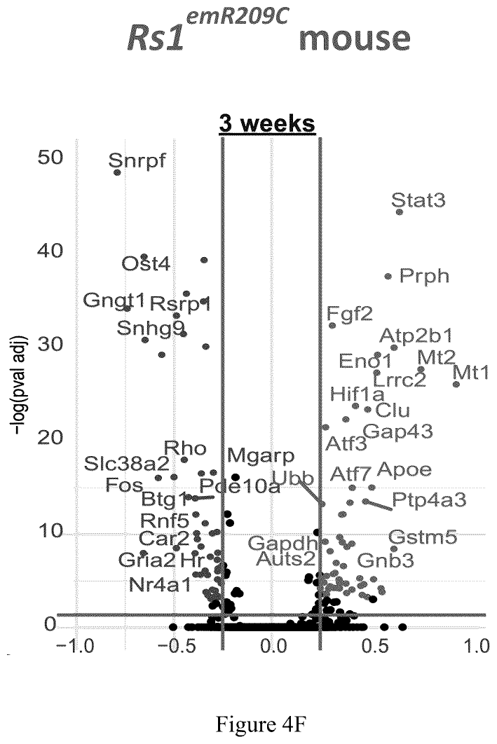

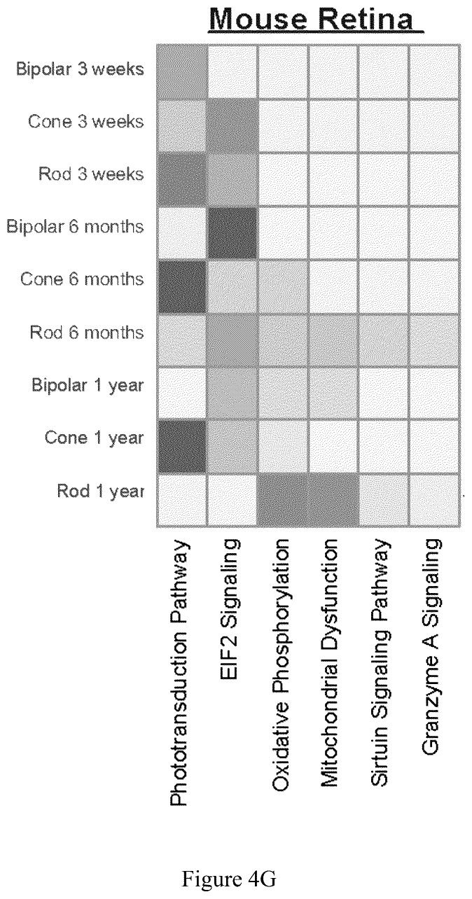

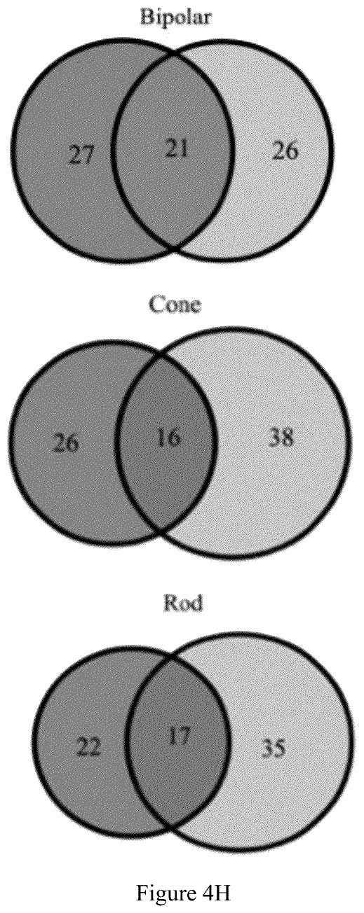

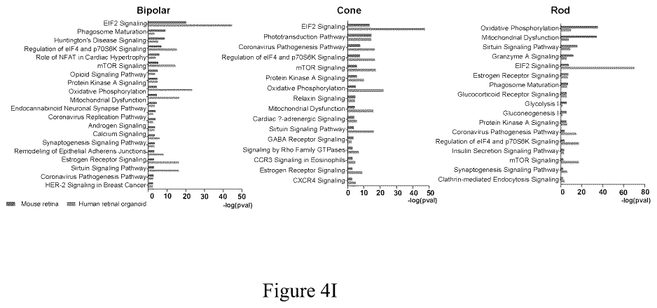

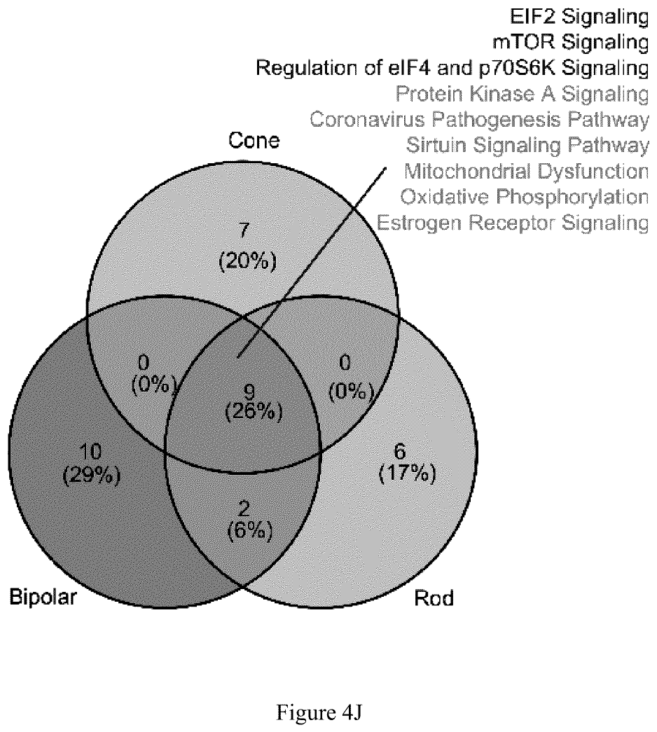

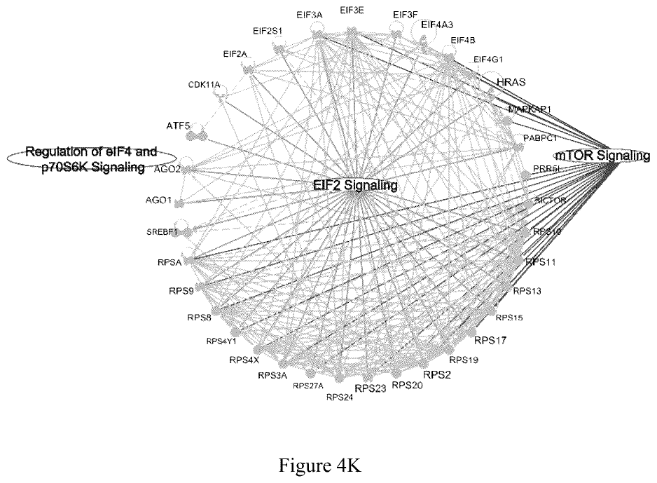

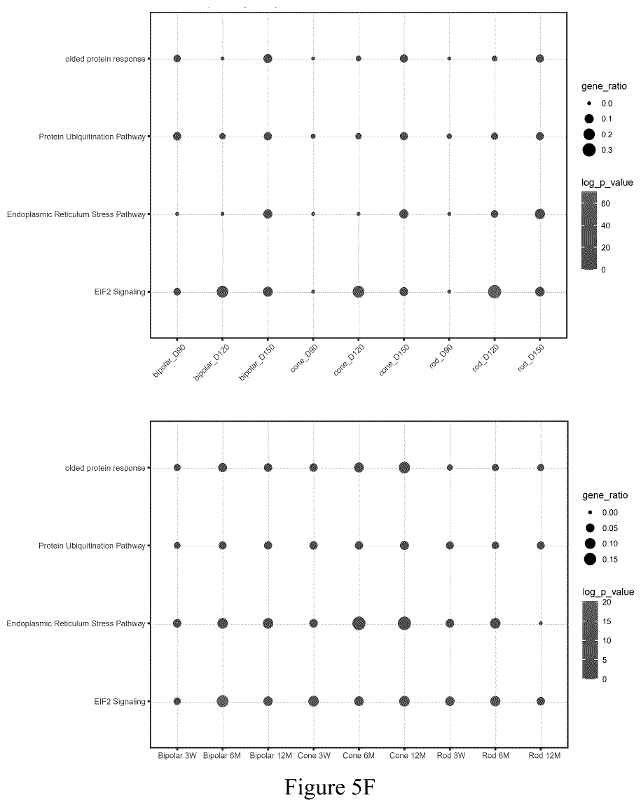

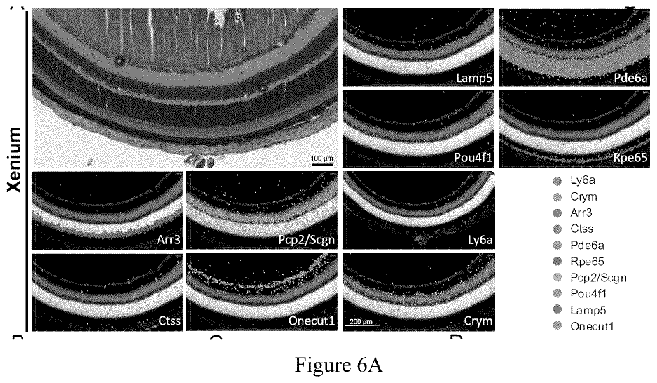

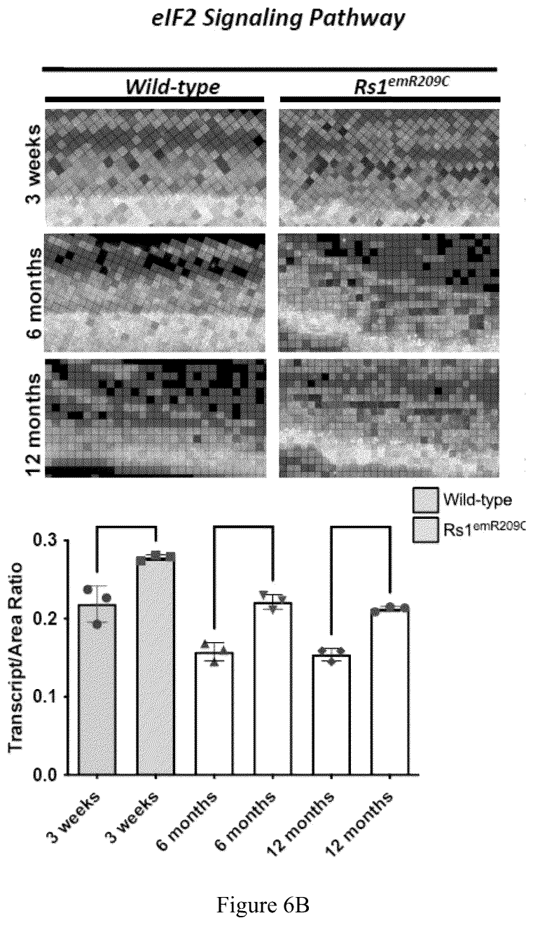

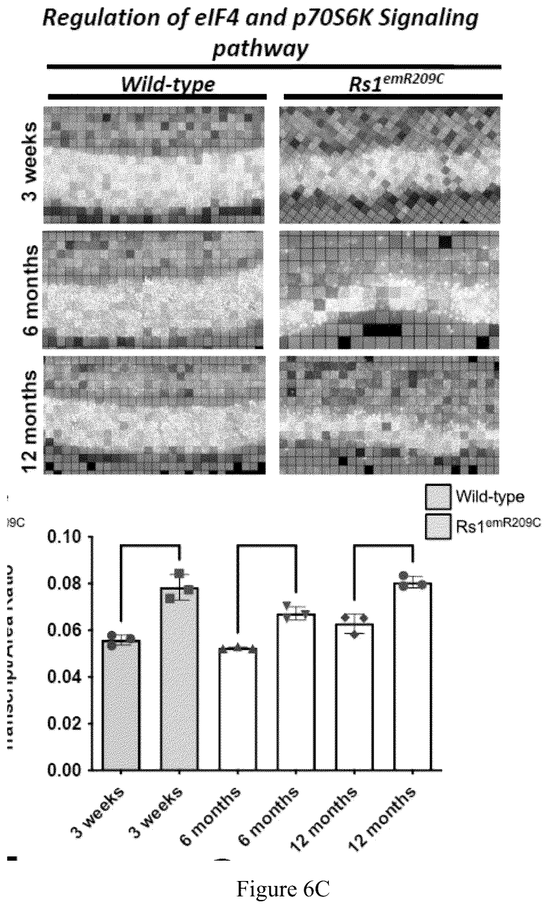

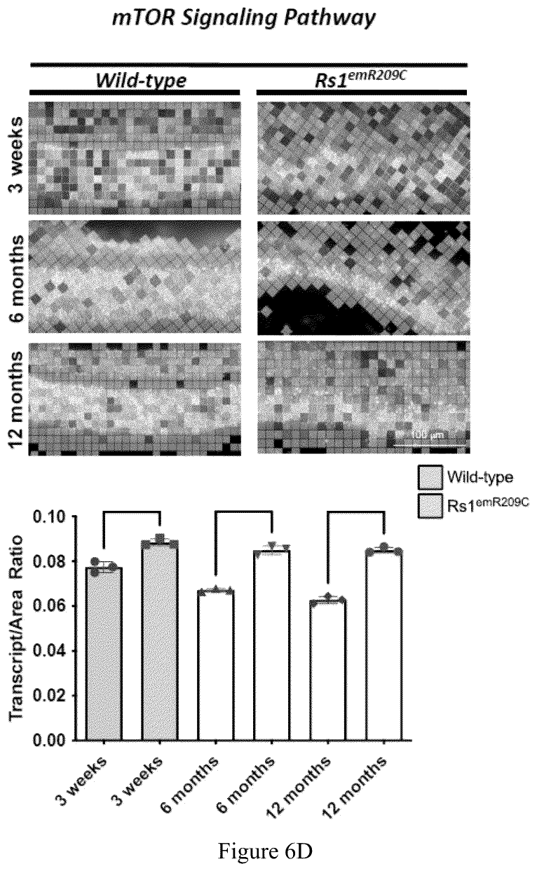

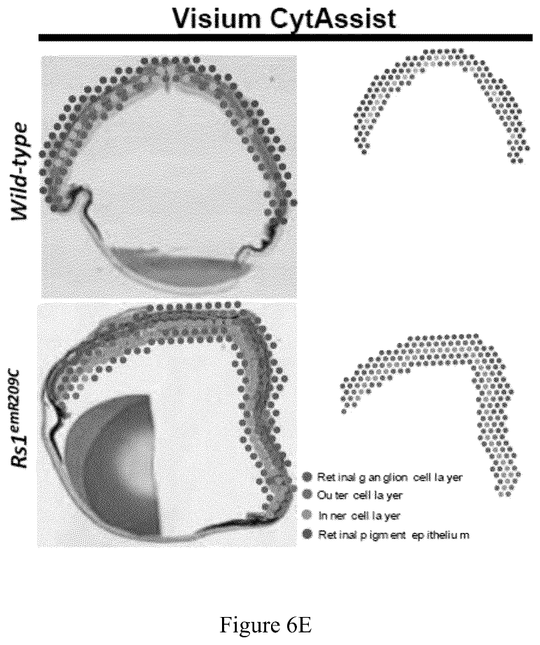

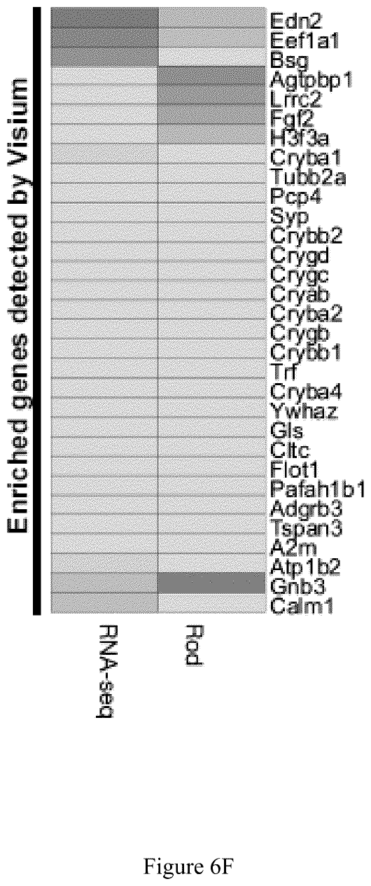

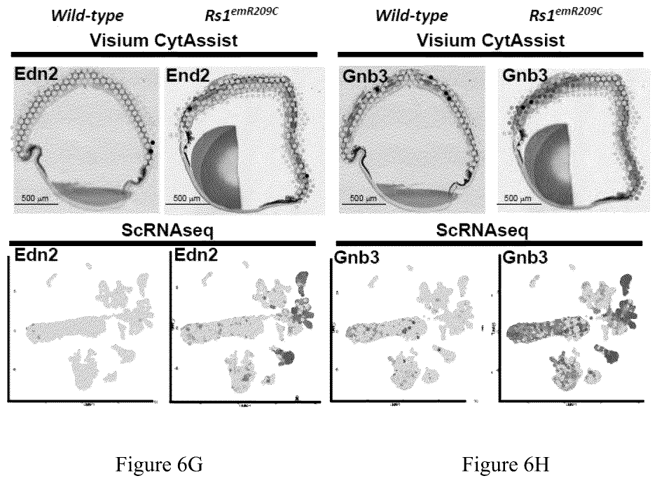

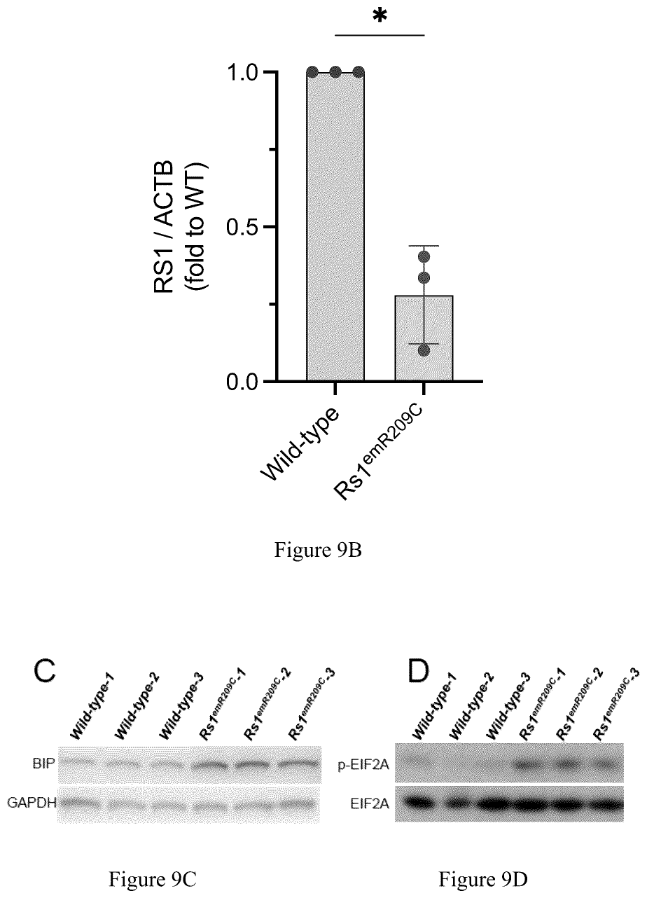

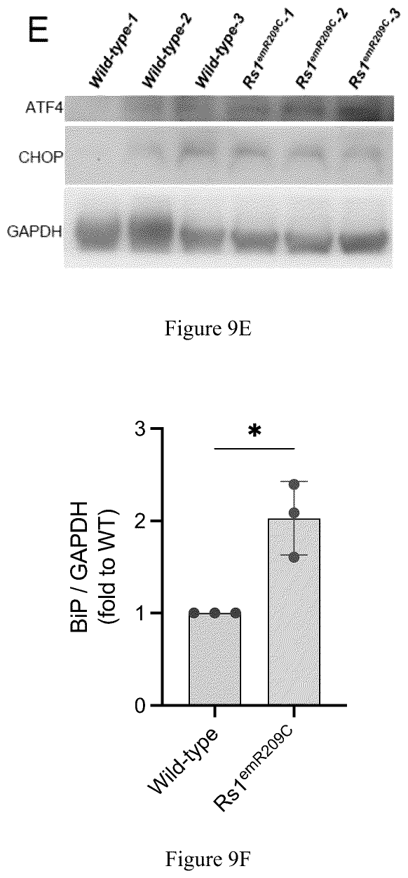

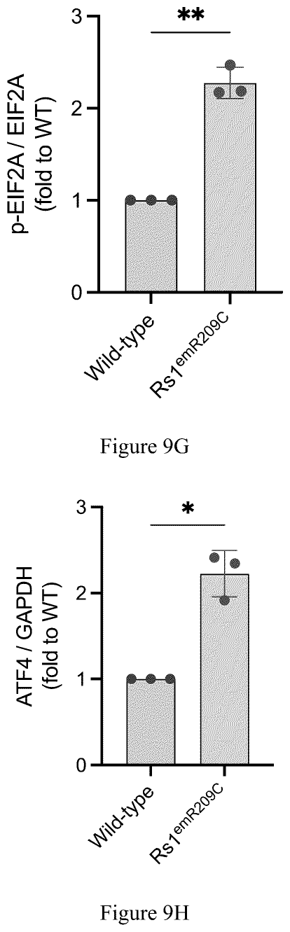

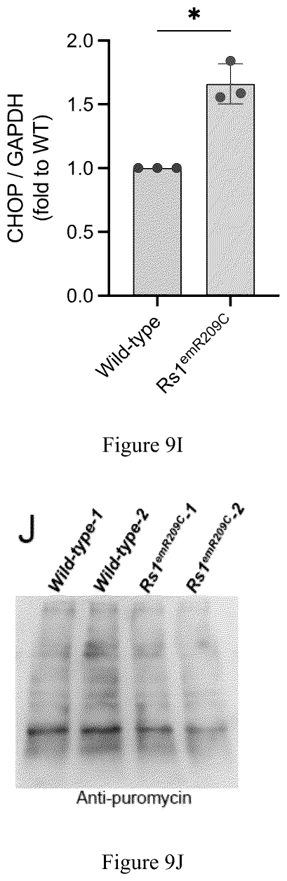

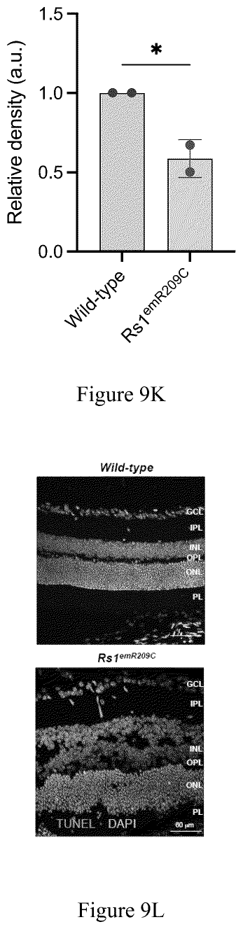

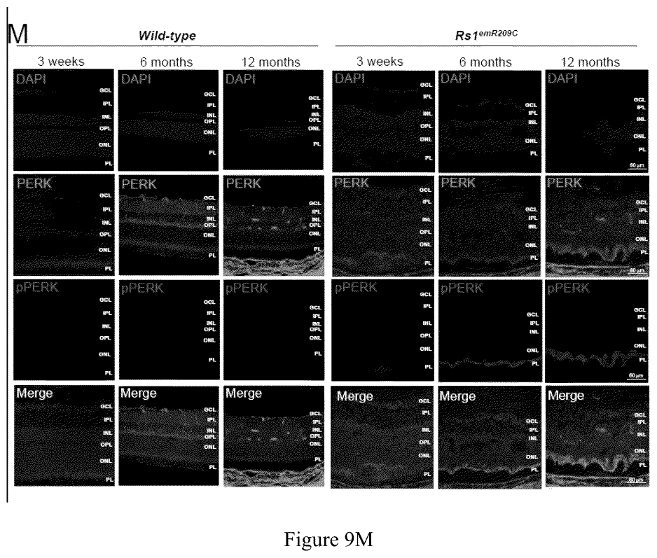

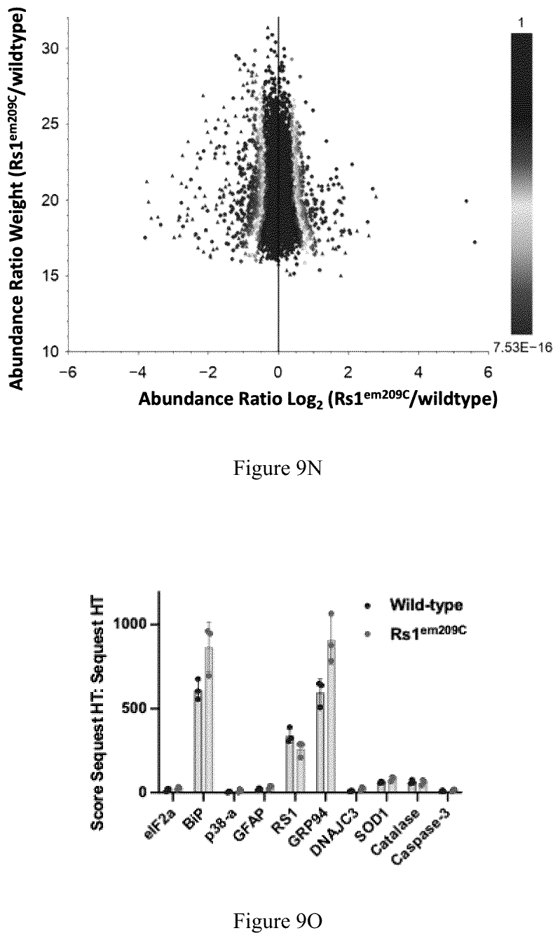

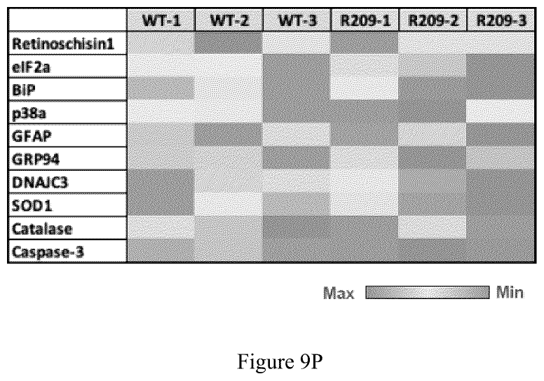

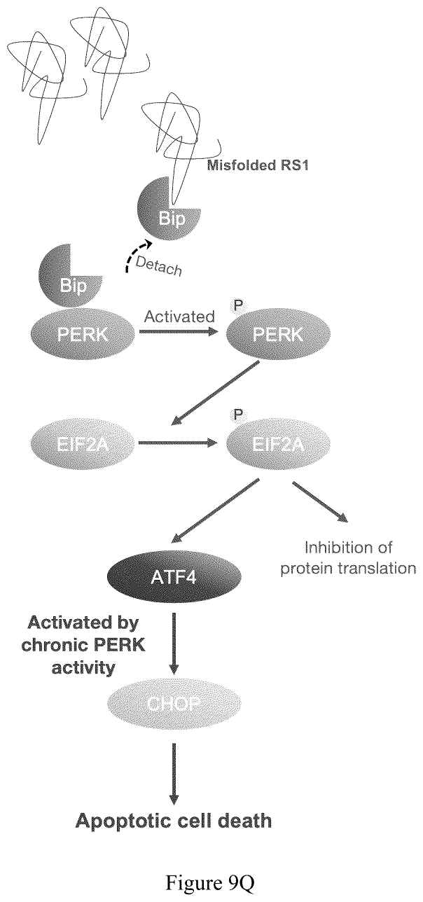

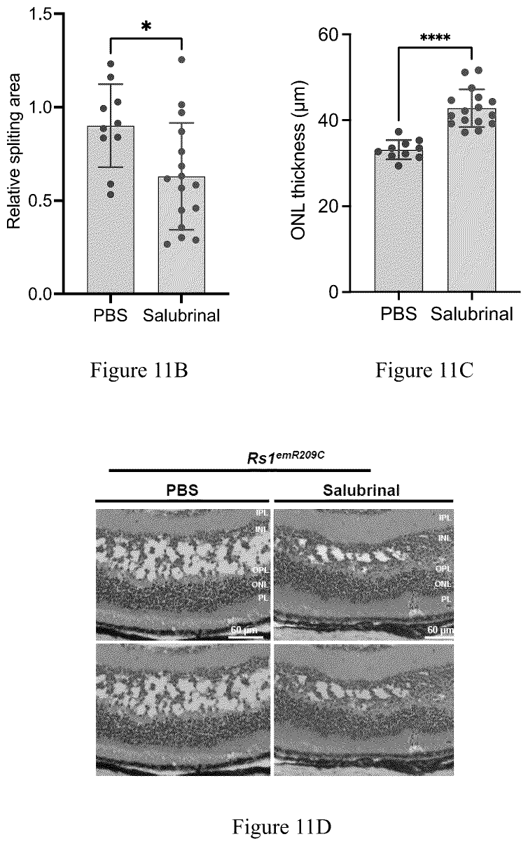

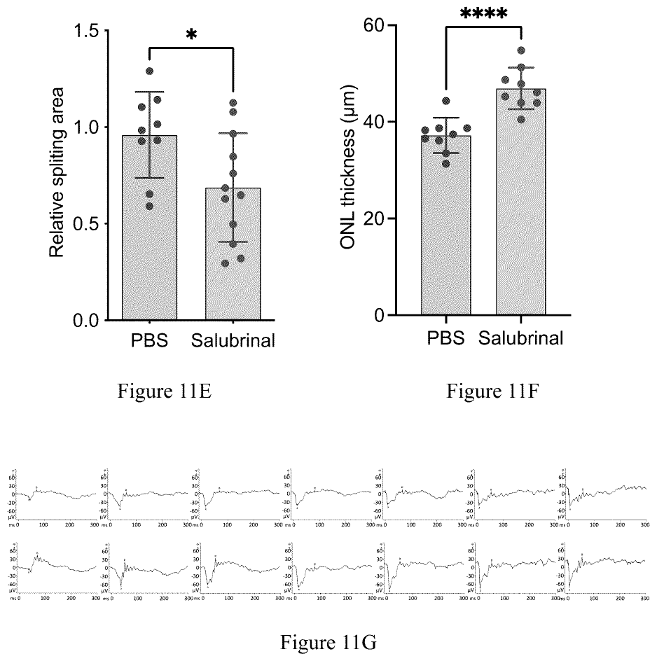

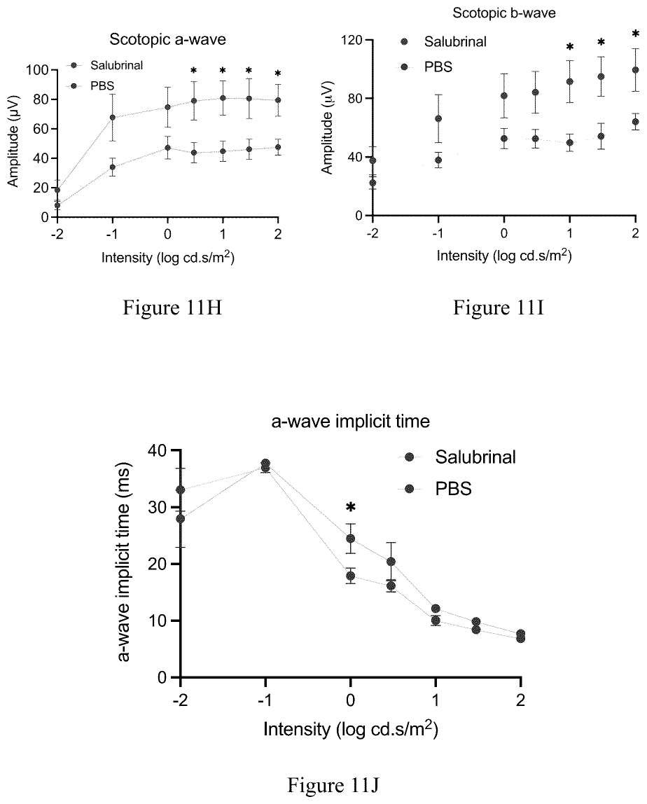

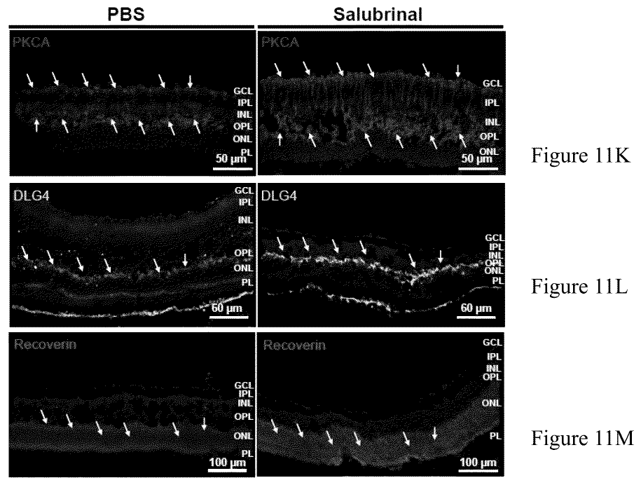

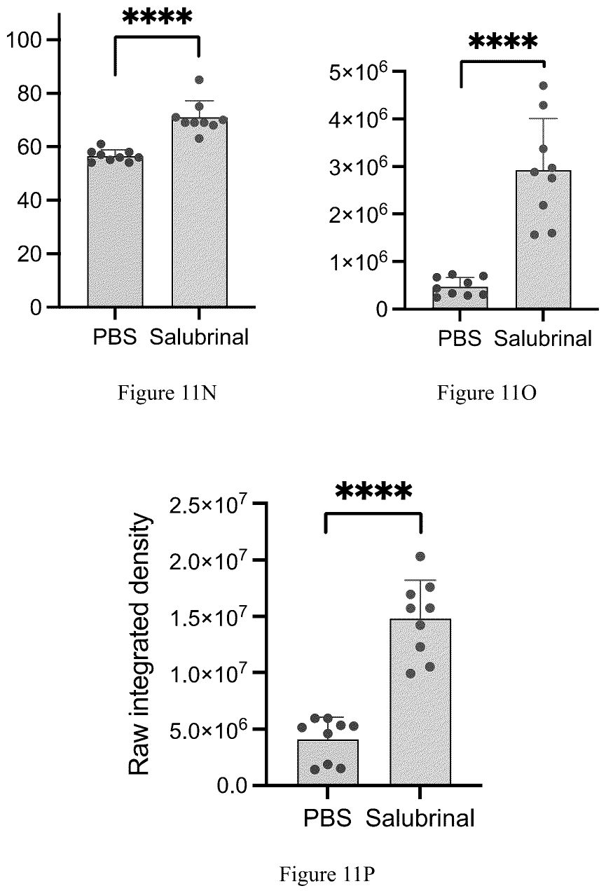

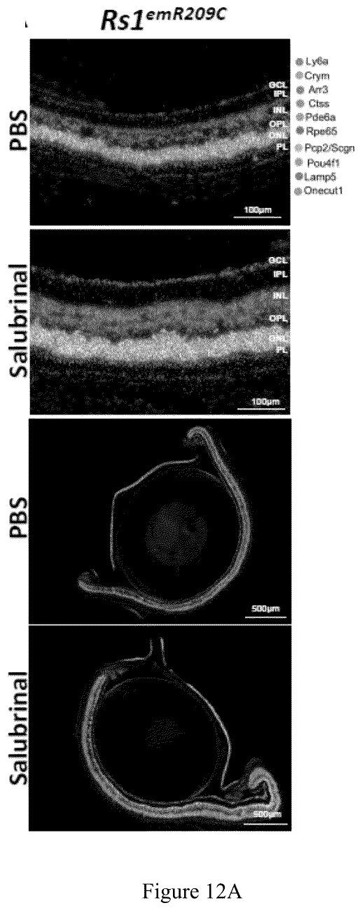

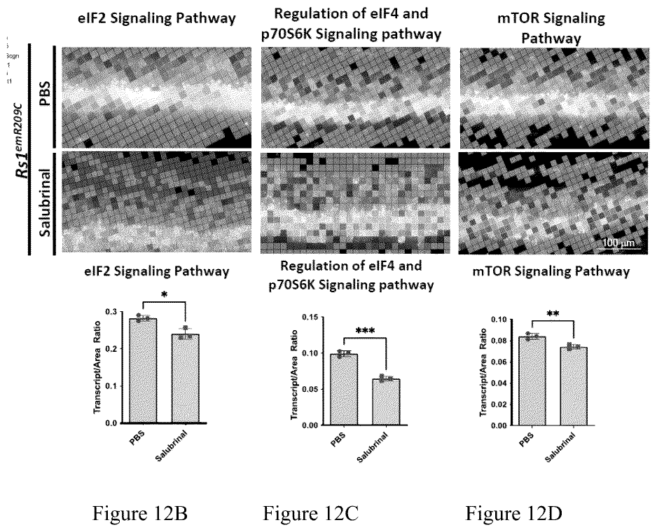

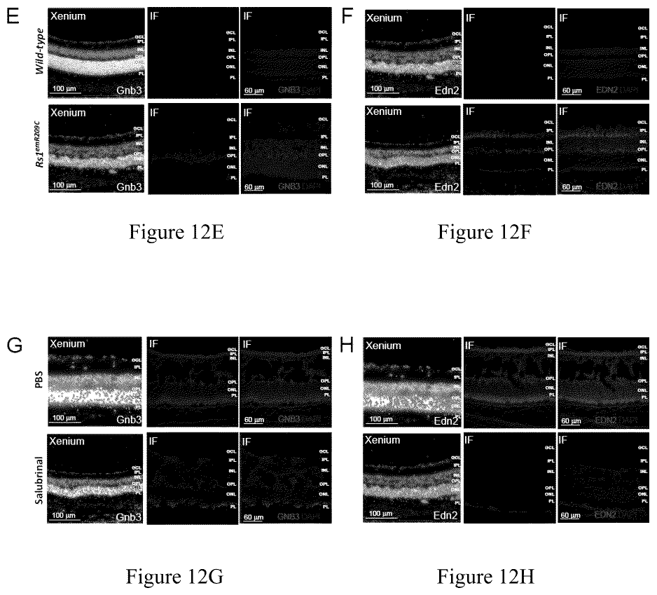

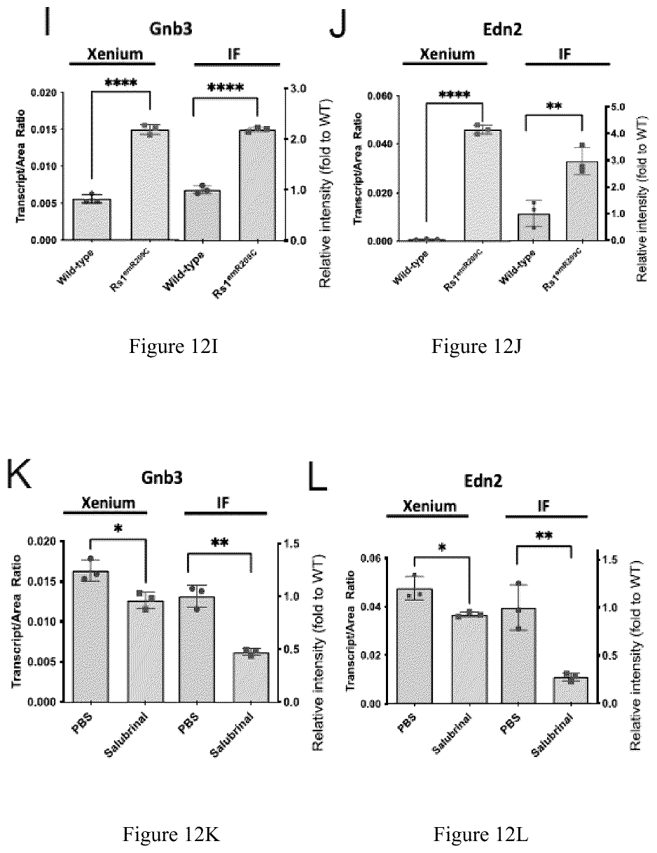

Advanced scRNA-seq and ST allow precise exploration of disease-specific transcriptomic signatures by analyzing differentially expressed genes at the single-cell level and within their spatial context [14, 20]. The eye consists of numerous small, intricate components that work together to focus on objects and transmit sensory information to the brain. Notably, the combination of scRNA-seq and ST was employed to elucidate the transcriptomic mechanisms underlying retinal development [21]. In the present study, we employed a multimodal approach combining ST and scRNA-seq and integrated transcriptomic data from Rs1_{emR209C} mouse retinas and patient iPSC-derived retinal organoids, both harboring the same patient-specific RS1 point mutations p.R209C. Using this multimodal spatiotemporal platform, we demonstrated that eIF2α signaling, the most enriched and highly conserved pathway, was chronically activated during disease progression in both in vivo and in vitro models (FIGS. 4 and 6). Through Western blots and LC/MS-based proteomics, we found that this chronic eIF2α signaling activation is accompanied with the failure of RS1 protein folding, impaired protein synthesis, and ER stress induced apoptosis (FIG. 9). Salubrinal, a small molecule inhibitor that targets the GADD34/protein phosphatase 1 complex to alleviate ER's workload and specifically inhibit ER stress-induced apoptosis [22-23], was used to therapeutically target the chronic activation of eIF2α signaling, demonstrating remarkable efficacy in mitigating retinoschsis and impaired electroretinogram responses (FIG. 11). Spatiotemporal transcriptomics validated the restoration of chronically enriched pathways after salubrinal treatment (FIG. 12). Importantly, salubrinal treatment improved the integrity of bipolar cells across the IPL, INL and OPL layers, increased postsynaptic neuron density in the OPL, and attenuated photoreceptor loss in the OPL layers in Rs1_{emR209C} retinas (FIG. 11). The combination of AAV-based Rs1 delivery and salubrinal demonstrated synergistic efficacy in alleviating XLRS-like phenotypes within 1 month.

The unfolded protein response (UPR) is triggered by ER stress and initiates when glucose regulated proteins (GRPs) detach from three key ER transmembrane sensors: IRE1, ATF6, and PERK. In the UPR pathways, PERK plays a critical role by phosphorylating eIF2α [28]. Under normal conditions, this phosphorylation triggers a protective response by broadly reducing overall protein synthesis [29]. However, when ER stress becomes prolonged or severe, phosphorylated PERK can no longer restore proteostatic balance and shifts towards a pro-apoptotic pathway by activating the pro-apoptotic transcription factor CHOP, ultimately leading to apoptotic cell death [30]. Chronic ER stress has been implicated in various neurodegenerative diseases, including Alzheimer's [36] and Parkinson's disease [37]. However, as an early-onset retinal degenerative disease, the role of chronic ER stress in XLRS has not yet been reported. Using non-reducing gels, we observed the formation of misfolded RS1 protein in Rs1_{emR209C} retinas. Western blot and LC/MS proteomics analysis revealed an enrichment of GRPs, such as BiP and GRP94 (FIG. 9). The accumulation of GRPs is widely recognized as a trigger for UPR pathways, including the PERK/eIF2α branch. In Rs1_{emR209C} retinas, the prolonged accumulation of misfolded RS1 protein may impair the eIF2α pathway's ability to maintain proteostasis, leading to CHOP activation and ER stress-associated apoptosis. Our findings of enriched CHOP protein levels and increased TUNEL+ cells in Rs1_{emR209C} retinas (FIG. 9) support this interpretation: the chronic accumulation of misfolded RS1 proteins contributes to excessive ER stress, ultimately activating apoptotic cell death mechanisms. Salubrinal treatment that can ease ER stress, restore the proteostatic balance, and mitigate ER stress-induced apoptosis significantly alleviated the severity of XLRS-like phenotypes and improved sensory transmission in Rs1_{emR209C} retinas (FIG. 11). The findings of targeting study supported that chronic ER stress accumulation, the prolonged activation of eIF2α signaling, and ER stress-associated apoptosis serve a critical role in the disruption of retinal architecture, progressive retinoschisis, and impaired visual function in Rs1emR209C retinas. Other highly enriched pathways identified by our multiomic approach included the mTOR pathway and the eIF4 and p70S6K pathways, both of which respond to ER stress [38]. Gnb3 and Edn2, two other enriched factors, were associated with impaired phototransduction [26] and gliosis [27], respectively. Additionally, ST also showed that salubrinal suppressed the aforementioned enriched pathways, and the expression of Gnb3 and Fdn2 in Rs1emR209C retinas. It is worthwhile to investigate the chronic activation of ER stress/eIF2 signaling in regulating the expression of Gnb3 and Edn2, as well as their downstream interactions in progressive ocular disorders and severe retinal degeneration.

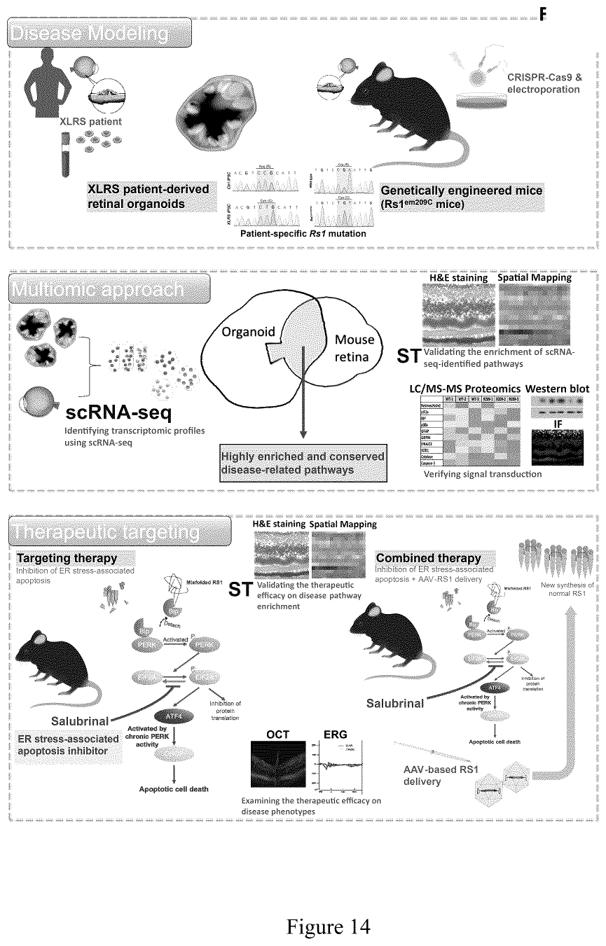

Although inconsistent outcomes in clinical trials of RS1 gene delivery have been attributed to potential systemic immunogenicity against AAV and vector-induced ocular inflammation [5], the exact mechanisms underlying the treatment's ineffectiveness in XLRS patients remain unclear. It is worth noting that in a single-center, consecutive, retrospective study, 132 participants with confirmed XLRS, followed between 1999 and 2020, were found to harbor 66 RS1 variants, 7 of which were novel, and exhibited diverse disease phenotypes [7]. In vivo studies using knock-in mice with various patient-specific mutations have demonstrated that genotype is a key determinant of XLRS-like phenotype severity [9b]. These clinical and in vivo findings both highlight the distinct role specific point mutations play in disease severity in XLRS. Our observations from the retinas of Rs1_{emR209C} knock-in mice showed that the causative p. R209C mutation disrupts the octameric structure of the RS1 protein, leading to the accumulation of misfolded RS1 protein. This, in turn, results in the chronic activation of eIF2α pathways, contributing to XLRS pathologies (FIG. 9). Remarkably, the combination of salubrinal and AAV8-based RS1 delivery significantly reduced both CHOP levels and the number of apoptotic cells, demonstrating a synergistic effect in mitigating retinoschisis and improving impaired ERG responses (FIG. 14). In contrast, AAV8-based RS1 delivery did not alter CHOP expression or prevent downstream ER stress-associated apoptotic cell death triggered by the misfolded RS1 protein. In addition, building on salubrinal's ability to alleviate ER stress and its associated apoptosis, our data suggest that therapeutically targeting the chronic activation of the eIF2α pathway, in combination with AA Vbased gene delivery, offers a novel and effective strategy for treating XLRS. Nevertheless, a limitation of the current study is that it focuses solely on XLRS models harboring p.R209C mutations. Further investigation is warranted to explore chronic ER stress-induced apoptosis and uncover additional biomolecular pathways involved in the pathogenesis associated with other RS1 point mutations in XLRS.

It could be concluded in the present invention that using two disease models with patient-specific gene mutations and identifying new transcriptional signatures through multiomics approaches can enhance our understanding of disease mechanisms and improve the development of treatment strategies in XLRS. In addition, future application of personalized medicine-based multimodal transcriptomic approach, not only enabling precise deciphering of the molecular pathogenesis of unexplored ophthalmic degeneration but also discovering new pharmaceutical strategies for precision-medicine-based therapeutics targeting for incurable severe hereditary diseases, including XLRS.

### Example

### Materials and Methods

### Generation of XLRS-Like Genetically Engineered Mice Carrying RS1 Mutations at c.625T (p.R209C)

The Transgenic Mouse Model Core Facility of the National Core Facility for Biopharmaceuticals, Ministry of Science and Technology, Taiwan, and the Gene Knockout Core Laboratory of National Taiwan University Centers of Genomic and Precision Medicine provided all the necessary techniques for the production of Rs1emR209C mice. The generation of these mice involved the utilization of CRISPR/Cas9 technology. The generation of Rs1emR209C mice with mutation of the specific nucleotide in the Rs1 gene involved the utilization of CRISPR/Cas9 technology. For Rs1emR209C mice, the sgRNA was designed to guide Cas9 cleavage in a targeted sequence of exon 6 of the Rs1 gene. The mutation changed the amino acid arginine (codon CGA) at #209 to cysteine (codon TGT) in the Rs1 gene. Selection of the sgRNA sequences following the online resources, including the sgRNA Designer: CRISPRko and Cas-OFFinder. The used sgRNAs were <3 mismatches and <25 off-target sites. The sgRNA target sequence with PAM sites (NGG) was 5′-GGCATGTCCGAATTGCCATC-3′ (SEQ ID NO: 1). The single-stranded ODN sequence was 5′-TCCCCCCAAAGCTCTCCCTGCAAGTGACAGAACTGAGCTGAAATAGACAT CAGGCACACTTGCTGGCACACTCAAGCAGCTCCATTCGAATGGCAATACA GACATGCCAGCCTAGAGGGATCAGTCG-3′ (SEQ ID NO: 2) and was produced by Integrated DNA Technologies (IDT). Preparation of sgRNA and Cas9 RNA for electroporation followed the commercial protocol, AmpliCap-Max™ T7 High Yield Message Maker kit (CELLSCRIPT® C-ACM04037). Electroporation was performed on fertilized eggs from C57BL/6J mice. After in vitro fertilization, the zygotes (1-cell stage) were subjected to electroporation with Cas9, single guide RNA (sgRNA), and ssODN. The zygotes (2-cell stage) were subsequently transferred to the foster mother, after which the founder mice were obtained. The founder mice were backcrossed to C57BL/6 mice for three generations to minimize off-target CRISPR/Cas9 gene editing changes in each line. Gene sequences of founder mice were validated by PCR, TA cloning, and sequencing. The PCR conditions for genotyping were 95° C. for 5 min, followed by 40 cycles of 95° C. for 30 sec, 58° C. for 30 sec and 72° C. for 30 sec, and a final extension at 72° C. for 7 min. For Rs1emR209C mice, the PCR primer sequences were

(SEQ ID NO: 3)
5′-GACTAGGCTTCCTCTTTCTCTTT-3′;
(SEQ ID NO: 4)
5′-AGATATAGCCCCATTCATCCC-3′

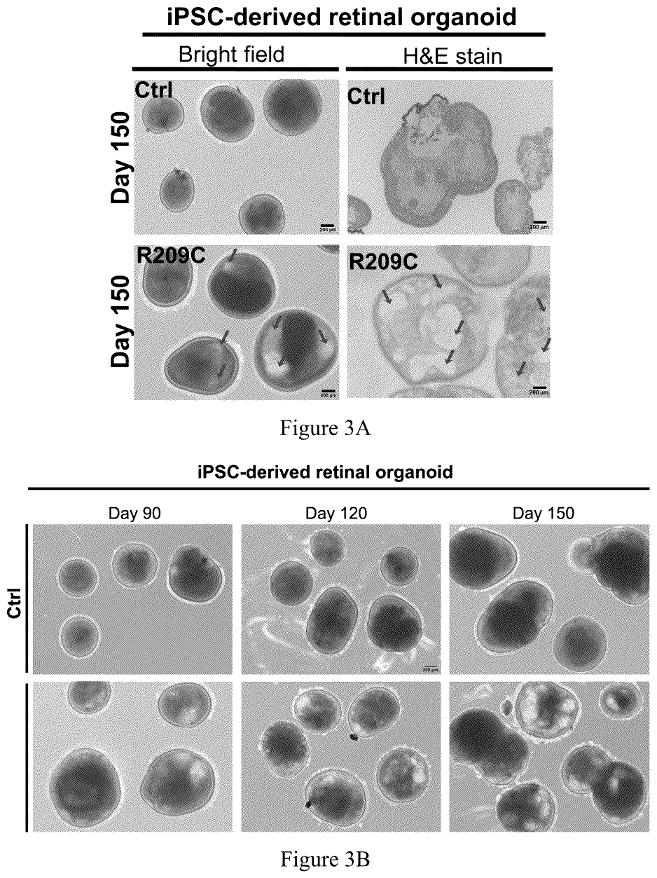

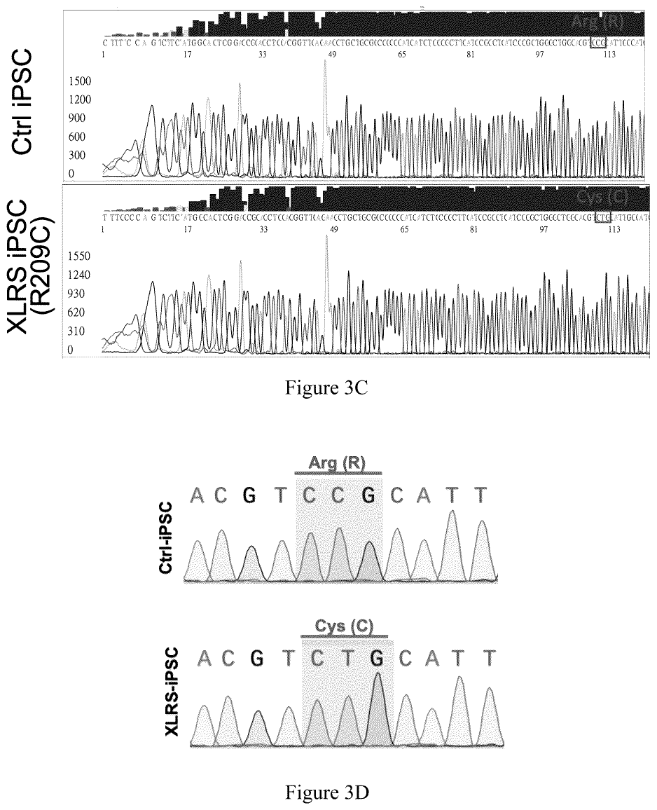

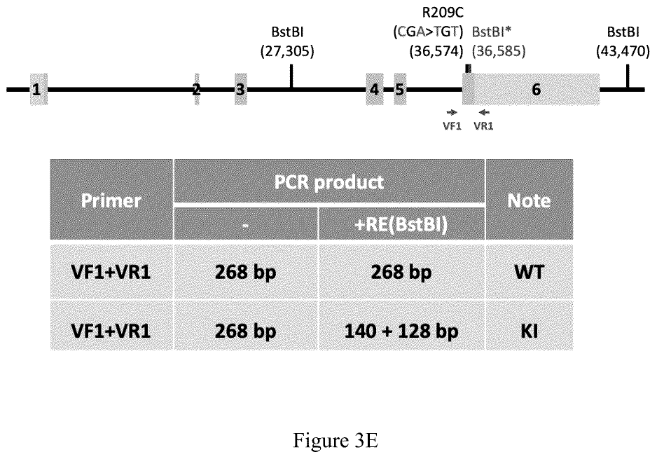

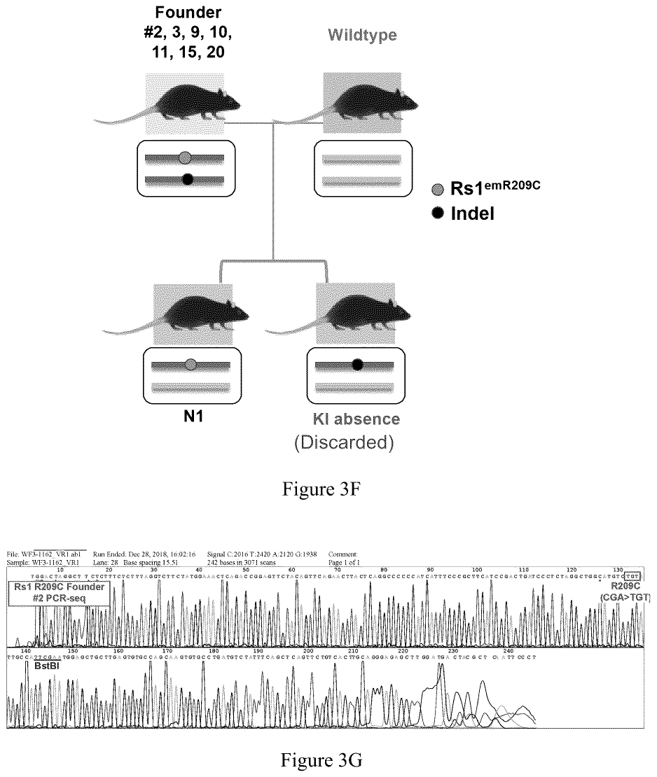

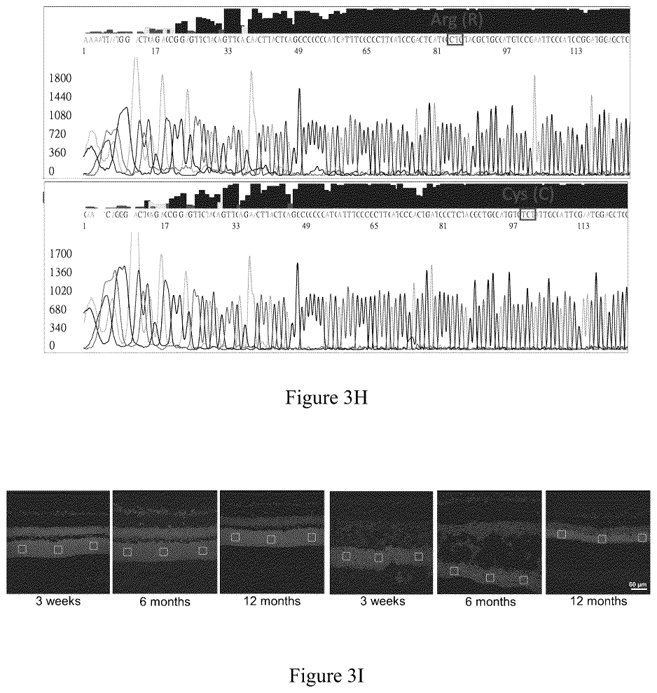

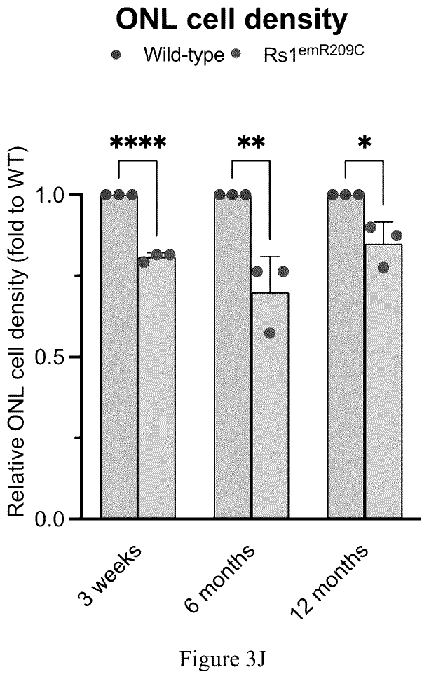

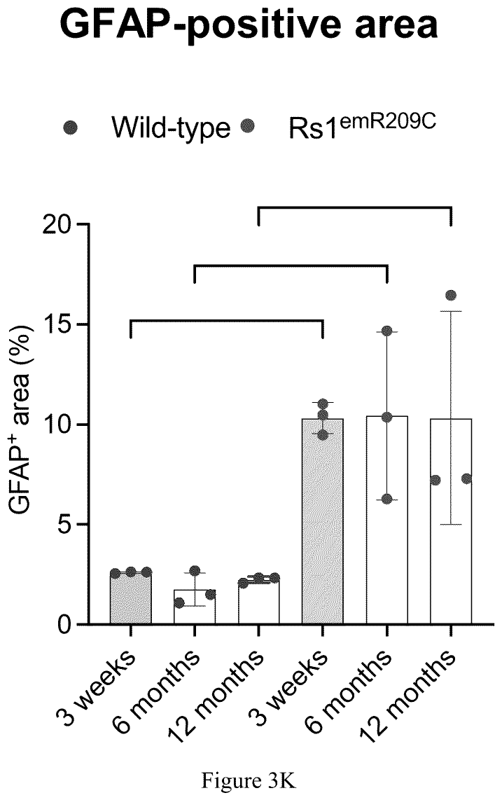

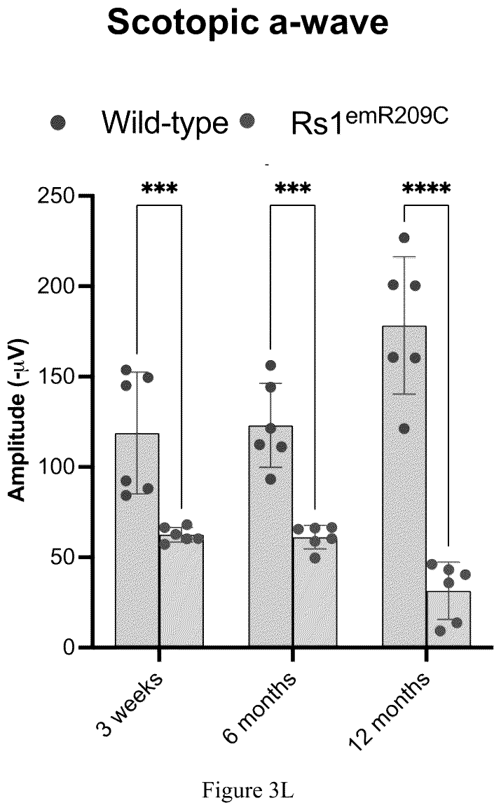

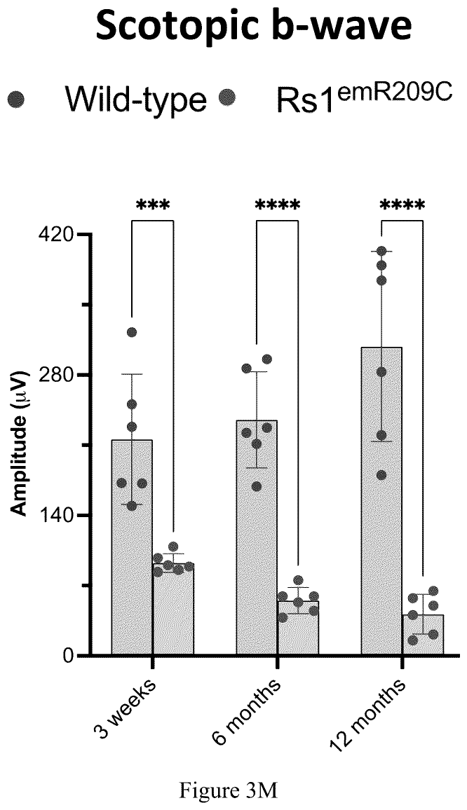

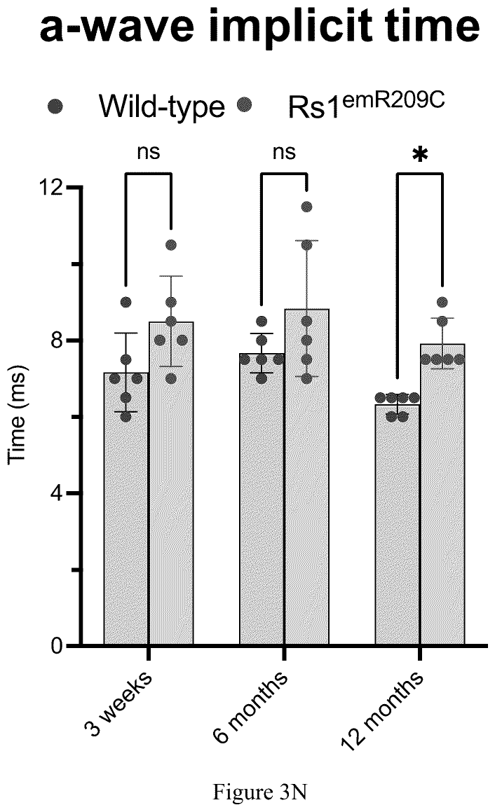

Protocol of TA-cloning followed by the T3 Cloning kit (ZGene Biotech Inc., #CT301-02). Finally, 4 male and 2 female founders for Rs1emR209C mice were selected. To minimize the off-target effect induced by CRISPR/Cas9 technology, the selected founders were individually backcrossed to C57BL/6 wild-type mice for 3 generations and then intercrossed to obtain homozygous knock-in mice for further experiments. Finally, mice with a knock-in patient-specific RS1 mutation were generated. (FIG. 3E-3G).

Maintenance and Differentiation of Human iPSCs into Retinal Organoids

Human induced pluripotent stem cells (hiPSCs) were maintained on Geltrex coated dishes in StemFlex medium (Gibco), adhering to the manufacturer's instructions. The cells were regularly passaged every 5-7 days upon reaching an approximate confluence of 80%. For the generation of retinal organoids, a previously established method was utilized with minor modifications [3]. In a nutshell, hiPSCs were detached using Vercene (Gibco) and converted into single cells. These cells were then cultured in a low attachment dish, along with a mixture of StemFlex and neural induction medium (NIM), supplemented with 10 μM ROCK inhibitor Y27632 (Sigma). The NIM consisted of DMEM/F12, 1% N2 supplement, 1×NEAAs, and 2 μg/ml heparin (Sigma). This culture setup initiated the formation of embryonic bodies on Day 0. Gradual transition to NIM was achieved by adjusting the medium ratio to mTeSRI/NIM on Day 3 and Day 5. On Day 7, the embryonic bodies (EBs) were transferred to six-well plates with NIM supplemented with 10% FBS. By Day 16, the medium was switched to retinal differentiation medium (RDM) consisting of DMEM/F12, 2% B27 supplement without vitamin A, 1×NEAAs, and penicillin/streptomycin. From Days 18 to 25, the central portions of neural clusters were manually separated and further cultured in suspension with RDM, allowing the formation of three-dimensional optic cup structures. Around Days 30 to 40, the retinal organoids were manually isolated. For long-term suspension culture, the medium was supplemented with 10% fetal bovine serum, 100 mM Taurine, and 2 mM GlutaMAX, starting from Day 35. From Days 60 to 90, the culture medium was supplemented with 1 μM retinoic acid to facilitate photoreceptor maturation. After Day 90, N2 supplement replaced B27 in the medium. The cell culture medium was regularly changed every 2-3 days until the desired developmental stage was achieved.

### Sanger Sequencing

Mutational detection of the Rs1 gene was performed following a previously described protocol with slight modifications. Genomic DNA was isolated from the tail of wild-type and Rs1emR209C mice. The primer pairs used for screening the mutation site of the Rs1 gene were designed as follows:

(SEQ ID NO: 3)
5′-GACTAGGCTTCCTCTTTCTCTTT-3′
and
(SEQ ID NO: 5)
5′-AAATCCTTATTGGCATTGAATCC-3′

The size of the PCR products was 268 bp. The sequencing products were analyzed on an ABI PRISM 3700 Genetic Analyzer (Applied Biosystems).

### Optical Coherence Tomography (OCT)

The MICRON IV imaging system (Phoenix-Micron) was utilized for OCT imaging to monitor retinal structural changes and cavity formation. Prior to imaging, mice were subjected to overnight dark adaptation and then anesthetized using 2% to 4% isoflurane inhalation. Topical tropicamide and phenylephrine were administered to achieve pupil dilation. Rectangular volume scans were performed, collecting a total of 100 B-scans. These scans covered an area centered on the optic nerve (ON) head. Additionally, two linear B-scans, averaging 30 frames each, were acquired from the nasal to temporal pole through the ON head.

### Electroretinogram (ERG)

Full-field ERGs were performed on male Rs1emR209C and wild-type mice to assess retinal function. The experiments were conducted using a Celeris system (Diagnosys). Mice were dark-adapted overnight and anesthetized by inhalation of 2% to 4% isoflurane. Pupil dilation was achieved with topical tropicamide and phenylephrine. Gold loop or wire electrodes were placed on the cornea. For the differential electrode, a gold wire was employed, and the tail was equipped with a connected ground wire. In the dark-adapted ERG, flash intensities ranging from −2 to 2 log cd·s/m2 were used. Responses were frequency filtered, and a-waves and b-waves were measured. The bwave-to-a-wave ratio (b/a) was calculated to assess the effects on the isolated postsynaptic response. The ERG signals were amplified, filtered, and analyzed using Espion software (Diagnosys). Throughout the recording sessions, the body temperature was monitored, and lubricating eye gel was administered to prevent corneal dryness.

### ScRNAseq Library Preparation and Sequencing

Retinas of wild-type and Rs1emR209C mice at 3 weeks, 6 months and 12 months were harvested for scRNAseq. After dissection, the retinas were initially immersed in Hanks' balanced salt solution (HBSS, Sigma), followed by dissociation of retinal cells using the MACS Neural Tissue Dissociation Kit for postnatal neurons (Miltenyi Biotec, 130-094-802) as per the manufacturer's instructions. Subsequently, the dissociated cells were filtered through a 40 μm nylon cell strainer, and washed with washing buffer (1×HBSS, 0.04% BSA). In each sample, a total of 16,000 retinal single cells (at a density of 1000 cells/μm) were resuspended in a washing buffer and prepared for scRNAseq library construction. The library construction for scRNAseq was carried out using the Chromium Next GEM Single Cell 3′ Kit v3.1 on a Chromium Connect instrument (10× Genomics). Subsequently, the libraries were sequenced on an Illumina NovaSeq 6000 platform with a target of 20,000 reads per cell.

ScRNAseq data processing The demultiplexing of sequences from each individual Illumina sequencing dataset was carried out using bcl2fastq v2.20.0.422 (Illumina). Subsequently, the processed sequencing reads were analyzed using 10× Genomics Cell Ranger version 6.0, with the reads aligned to the mouse reference genome mm10 version 3.0.0. To ensure data quality, various steps such as QC filtering, clustering, dimensionality reduction, visualization, and differential gene expression analysis were performed using Seurat v4.04 with R v4.1.0. Cells were filtered out based on the following criteria: (1) exclusion of doublets, (2) exclusion of cells expressing 15% or more mitochondrial genes, (3) exclusion of cells with fewer than 200 expressed genes (considered as droplets or cellular debris), and (4) exclusion of cells expressing 7,500 or more genes. Each dataset was then subjected to log-normalization using Seurat's NormalizeData function with default parameters. Using Seurat's FindVariableFeature function, we identified 2,000 features in the dataset that displayed significant cell-to cell variation. Subsequently, we applied linear regression against the number of reads to scale the data using Seurat's ScaleData function with default parameters. The variable genes were then projected onto a lower-dimensional space using principal component analysis (PCA) through Seurat's RunPCA function with default parameters. The number of principal components was determined by examining the plot of variance explained, and a value of 30 principal components (Npcs=30) was selected. Datasets were integrated using the Seurat integrated default workflow. A shared nearest neighbor graph was constructed based on the Euclidean distance in the low-dimensional subspace using Seurat's FindNeighbors with dims=1:30 and default parameters. Integrated datasets then underwent nonlinear dimensional reduction and visualization using UMAP. Clusters were identified using a resolution of 0.5 and the Leiden algorithm for the integrated datasets. Cell types were assigned to each cell based on their highest cell type module score created from wild-type controls.

### Differential Expression Analysis and Functional Enrichment

Differential expression analysis was performed using Seurat's FindMarker function to identify DEGs between Rs1emR209C and wild-type groups and between different samples and sample groups within each cluster. DEGs were selected based on a significance threshold of p-value <0.05. Pathway enrichment analysis was conducted using Ingenuity Pathway Analysis (IPA) to further understand the biological significance of DEGs. This involved annotating gene lists with functional categories and pathways and examining enriched processes in each cluster. The strength of associations was represented by −log FC. Pathways with an adjusted p-value of <0.05 were considered significant. Moreover, to investigate the potential functions and interactions of the DEGs, GO, GSVA, and IPA were performed.

### Bulk RNA Sequencing and Analysis

To extract RNA from retinas of wild-type and Rs1emR209C mice at 3 weeks, 6 months, and 12 months of age, TRIzol (Invitrogen) was used following the provided instructions. The purified RNA was employed for constructing sequencing libraries using the TruSeq Stranded mRNA Library Prep Kit (Illumina, San Diego, CA, USA) according to the manufacturer's guidelines. Initially, mRNA was isolated from 1 μg of total RNA using oligo(dT)-coupled magnetic beads and fragmented at an elevated temperature. Subsequently, first-strand cDNA was synthesized using reverse transcriptase and random primers. Double-strand cDNA was generated, and DNA fragments underwent adenylation at the 3′ ends before ligating adaptors. The resulting products were enriched by PCR and purified using the AMPure XP system (Beckman Coulter, Beverly, USA). The quality of the libraries was assessed using the Qsep400 System (Bioptic Inc., Taiwan), and their quantity was determined with the Qubit 2.0 Fluorometer (Thermo Scientific, Waltham, MA, USA). The qualified libraries were then sequenced on an Illumina NovaSeq 6000 platform, generating 150 bp paired-end reads. The original image data were converted into sequence data through base calling, resulting in raw data or raw reads saved as FASTQ files. All FASTQ sequences were aligned to the mouse genome mm9 (GRCm37). For the alignment and quantification steps, Kallisto was used for file format conversion. DESeq2 (version 1.22.2) was employed for differential expression gene analysis. Genes exhibiting a 1.5-fold expression over the background with a q-value (FDR) less than 0.05 were considered as differentially expressed genes (DEGs) in each cluster.

### Spatial Transcriptomics

### Xenium In Situ

The custom gene panel for retinas was designed based on the scRNA-seq data.

Xenium In Situ Gene Expression technology was performed using the Xenium in Situ Gene Expression Reagent Kits (10× Genomics) following the manufacturer's instructions. Briefly, formalin-fixed, paraffin-embedded mice retina tissue sections (5 μm) were cut and placed on the Xenium Slides (10× Genomics, PN-3000941). After probe hybridization, ligation, and amplification, we immediately loaded the Xenium slide onto the Xenium Analyzer. Post Xenium, retina sections were stained with hematoxylin and eosin to obtain the histological data that can be combined with gene expression data from the same tissue section. Gene expression data was analyzed using Xenium Explorer software. Initially, we clustered the known gene sets, identifying corresponding cells and their cell types. Subsequently, we conducted a more in-depth analysis of the target pathogenic pathway. Each transcript was transformed into a heatmap, with darker colors indicating a higher transcript count. Comparisons were made across different samples.

### Cytassist Visium

The 5 μm sections of paraffin-embedded retinas from both wild-type and Rs1emR209C samples underwent deparaffinization, H&E staining, and imaging using a Nikon Eclipse T2 microscope, following the 10× Genomics protocol (CG000520). For the construction of the sequencing library, the Visium CytAssist FFPE Spatial Gene Expression 6.5 mm (Mouse; 10× Genomics) and the Visium CytAssist instrument (10× Genomics) were utilized. Subsequently, the libraries were sequenced on an Illumina NovaSeq 6000 platform. After library construction and sequencing, the Tissue Microarray (TMA) ‘count’ method in Space Ranger (v2.1.0; 10× Genomics) was employed to align the probe reads to the mouse reference genome (mm10) using a short-read probe alignment algorithm. The resulting count matrix, along with the accompanying H&E images, were further analyzed using the R package Seurat (v.4.3.0). The gene-count matrices were normalized using SCTransform and integrated into a single object for joint processing. Integration of datasets was performed using Seurat's default workflow for integration. A shared nearest neighbor graph was constructed based on the top 30 principal components (PCs) and default conditions using Seurat's FindNeighbors function, utilizing the Euclidean distance in the low-dimensional subspace. The expression levels of specific genes were visualized using the SpatialDimPlot and SpatialFeaturePlot functions. Differential expression gene analysis was conducted using the FindMarkers function.

### Immunohistochemistry

Immunohistochemistry staining was performed as previously described. The floating retinal organoids were collected, fixed with 4% paraformaldehyde for 30 min, paraffin-embedded, and sectioned. The mouse retinas were collected, fixed with Hartmann's fixative, paraffin-embedded, and sectioned. Paraffin-embedded retinal organoids and retinas were stained with H&E. For IF, mouse eyeballs were embedded in Tissue-Tek O.C.T. Compound (Sakura), and frozen sectioned using a cryostat (Leica). After blocking and permeating steps, the cells were incubated with primary antibodies and secondary antibodies. Images were acquired with an Olympus FV3000 laser scanning confocal microscope.

### TUNEL Staining

To detect retinal cell apoptosis, the TUNEL (terminal deoxynucleotidyl transferase dUTP nick-end labeling) In Situ Apoptosis Kit from Elabscience Biotechnology was employed, following the manufacturer's instructions. Initially, each section was treated with proteinase K and incubated at 37° C. for 10 minutes. Subsequently, TdT labeling solution was applied to each section and incubated at 37° C. for 30 minutes. The slides were then washed with PBS three times, with each wash lasting 5 minutes. In addition, frozen retina sections were stained with 4′,6′-diamino-2-phenylindole (DAPI) to facilitate the visualization of the cell nucleus.

### Western Blot

Mouse retinas were lysed using RIPA lysis buffer (Millipore) containing a protease inhibitor cocktail (Roche). Protein concentration normalization was achieved by utilizing Protein Assay Dye Reagent Concentrate from Bio-Rad. Equal amounts of proteins were denatured in either a 10% (reducing conditions) or 6% (nonreducing conditions) Bis-Tris precast polyacrylamide gel, along with an SDS mixture (10 mM Tris, pH 6.8, 1% SDS, 10% glycerol), with or without 4% β-mercaptoethanol. The gel was then transferred onto a membrane using transfer buffer (25 mM Tris, 192 mM glycine, and 20% methanol) for a duration of 2 hours. After blocking with 5% skim milk in TBST for 1 hour, the membranes were incubated overnight at 4° C. with primary antibodies targeting RS1 at a dilution of 1:1000. The membranes were blocked in 5% skim milk in TBST for 1 hour and incubated overnight at 4° C. with primary antibodies against RS1 (1:1000 dilution), p-EIF2A, EIF2A, ATF4, BiP, CHOP, GAPDH, and β-actin (all at a 1:20000 dilution). Following three washes with TBST, the membranes were incubated with HRP-conjugated secondary antibodies (1:1000 dilution in 5% BSA) for 60 minutes at room temperature. Subsequently, the membranes were washed three times with TBST, detected using Immobilon ECL Ultra Western HRP Substrate (Millipore), and imaged using UVP ChemStudio PLUS (Analytik Jena).

### Salubrinal Treatment

Rs1emR209C mice were intraperitoneally injected once daily starting at 3 weeks of age with 1 mg/kg salubrinal (Sigma) at a volume of 100 μL for one month. The vehicle group received an equal volume of phosphate-buffered saline (pH 7.2). Mice were monitored daily for any adverse effects.

### AAV-RS1 Delivery

The design of AAV-RS1 vector and the procedure to deliver AAV-RS1 were conducted based on a previously described protocol with modifications. Rs1emR209C mice at 3-week-old age were subjected to the intravitreal injection of 2×109 viral vector genomes per eye using a Hamilton syringe equipped with a 33-gauge needle. Prior to injection, animals were anesthetized with 2% to 4% isoflurane. Retinal structure and electrophysiological functions were assessed a month after the AAV-mediated RS1 injection.

Primers of qRT-PCR

The primers of qRT-PCR used in the present invention are given in Table 1.

TABLE 1
qRT-PCR PRIMERS

| | Forward primer | Reverse primer |
|---|---|---|
| SXBP1 | CTGAGTCCGAATCAGGTGCAG | GTCCATGGGAAGATGTTCTGG |
| | (SEQ ID NO: 6) | (SEQ ID NO: 7) |
| ATF4 | GTGGCCAAGCACTTGAAACC | GGAAAAGGCATCCTCCTTGC |
| | (SEQ ID BO: 8) | (SEQ ID BO: 9) |
| HSPA5 | TTCAGCCAATTATCAGCAAACT | TTTTCTGATGTATCCTCTTCACCA |
| | CT (SEQ ID BO: 10) | GT (SEQ ID NO: 11) |
| DDIT3 | CATACACCACCACACCTGAAAG | CCGTTTCCTAGTTCTTCCTTGC |
| | (SEQ ID NO: 12) | (SEQ ID NO: 13) |
| DNAJC | ACGCCTTTGACGGTGCCGATTA | AAGTCGCTGATGGCTTTCCTGG |
| 3 | (SEQ ID NO: 14) | (SEQ ID NO: 15) |
| GRP94 | GACCTTCGGGTTCGTCAGAG | AGCCTTCTCGGCTTTTACCC |
| | (SEQ ID NO: 16) | (SEQ ID NO: 17) |
| β-actin | TTGCTGACAGGATGCAGAAG | GTACTTGCGCTCAGGAGGAG |
| | (SEQ ID NO: 18) | (SEQ ID NO: 19) |
| LAMP1 | CAGCACTCTTTGAGGTGAAAAA | ACGATCTGAGAACCATTCGCA |
| | C (SEQ ID NO: 20) | (SEQ ID NO: 21) |
| LAMP2 | ATATGTGCAACAAAGAGCAGGT | TGCCAATTAGGTAAGCAATCACT |
| | (SEQ ID NO: 22) | (SEQ ID NO: 23) |
| SOD1 | CAGAAGGCAAGCGGTGAAC | CAGCCTTGTGTATTGTCCCCATA |
| | (SEQ ID NO: 24) | (SEQ ID NO: 25) |
| Catalse | GGACGCTCAGCTTTTCATTC | TTGTCCAGAAGAGCCTGGAT |
| | (SEQ ID NO: 26) | (SEQ ID NO: 27) |
| Caspase | TGCAGCATGCTGAAGCTGTA | GAGCATGGACACAATACACG |
| 3 | (SEQ ID NO: 28) | (SEQ ID NO: 29) |
| Fas | GCTGCAGACATGCTGTGGATC | TCACAGCCAGGAGAATCGCAG |
| | (SEQ ID NO: 30) | (SEQ ID NO: 31) |

### Information of Antibody

The information of the antibodies used in the present invention is given in Table 2.

TABLE 2
ANTIBODY INFORMATION

| Antibody | Species | Manufacturer | Cat. No. |
|---|---|---|---|
| anti-PKCa | Rabbit | Cell Signaling | 2056 |
| anti-DLG4 | Rabbit | Cell Signaling | 2507 |
| anti-GFAP | Rabbit | abcam | ab7260 |
| anti-RS1 | Mouse | abcam | ab167579 |
| anti-BiP | Rabbit | abcam | ab21685 |
| anti-EIF2A | Rabbit | Cell Signaling | 9722 |
| anti-Phospho-EIF2A | Rabbit | Cell Signaling | 9721 |
| (Ser51) | | | |
| anti-ATF4 | Rabbit | Cell Signaling | 11815 |
| anti-CHOP | Mouse | Cell Signaling | 2895 |
| HRP-conjugated anti- | Mouse | Proteintech | HRP-60004 |
| GAPDH | | | |
| anti-Puromycin | Mouse | Millipore | MABE343 |
| anti-PERK | Rabbit | Cell Signaling | 3192 |
| anti-phospho-PERK | Rabbit | Affinity Biosciences | DF7576 |
| (Thr982) | | | |
| atnti-GNB3 | Rabbit | Proteintech | 10081-1-AP |
| anti-EDN2 | Rabbit | NOVUS | NBP1-87942 |

### Statistical Analysis

The data are presented as the mean±SEM and were analyzed using Microsoft Excel. The statistical analysis was performed using Student's t-test or one-way ANOVA. A difference was considered significant when the p-value was less than 0.05.

### Results

### Modeling XLRS Using Patient-Derived Retinal Organoids and Genetically Engineered Mice

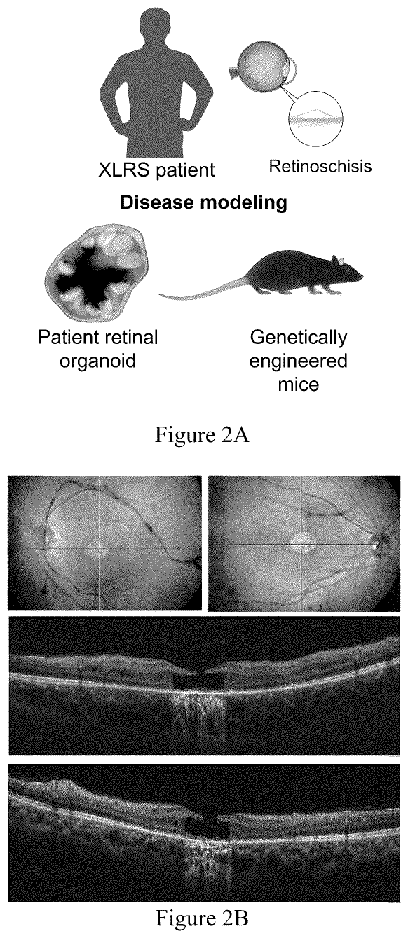

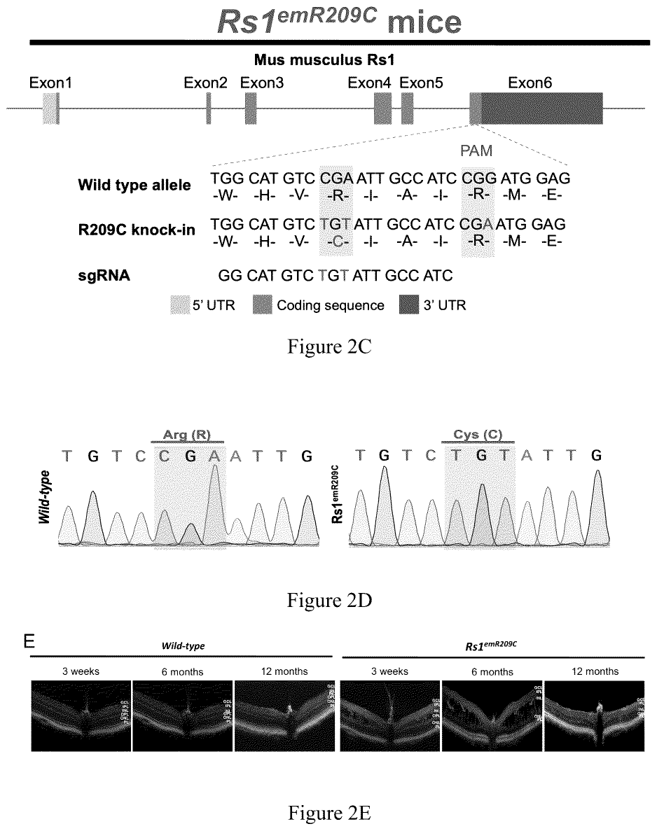

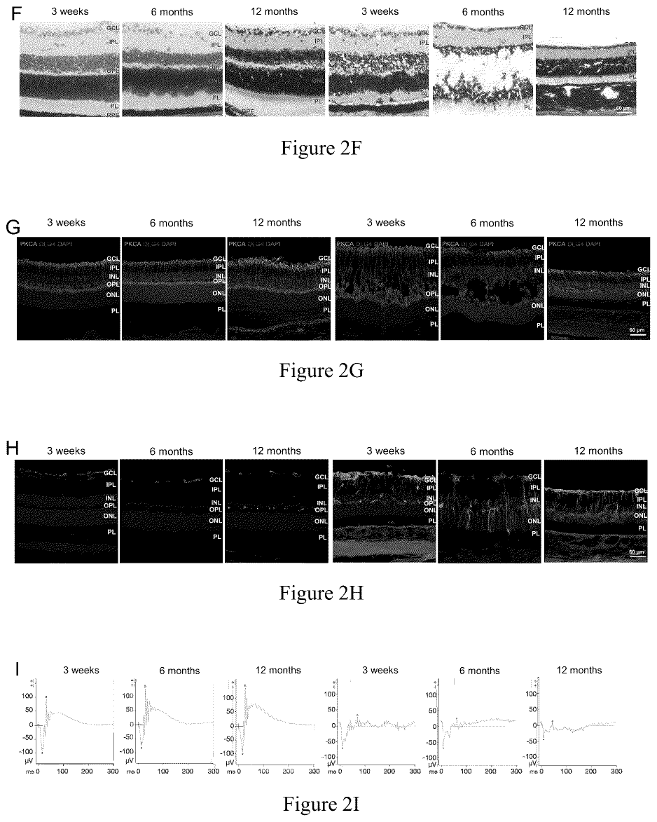

To establish XLRS-like models, we generated in vivo genetically engineered mice harboring the specific point mutations found in XLRS patients and used in vitro patient iPSC-derived retinal organoids carrying the same mutation, as described previously [10a]. We then employed multiomic approaches to collect transcriptomic signatures from both models, aiming to elucidate the molecular pathogenic mechanisms of XLRS (FIG. 2A). With respect to the in vitro XLRS model, we enrolled an XLRS patient who carried the c.625C>T (p. R209C) mutation and exhibited typical XLRS features, as described previously [10a]. The clinical findings of the ophthalmological survey and the patient's fundus photography showed macular atrophy and the characteristic “spoke-wheel” appearance (FIG. 2B; left eye, upper left; right eye, upper right). Optical coherence tomography (OCT) images revealed loss of the inner retina and outer nuclear layer (INL and ONL) in the fovea and mild retinoschisis at the nasal site (FIG. 2B; left eye, middle; right eye, bottom). We then collected peripheral blood mononuclear cells (PBMCs) from the XLRS patient and a healthy control donor to reprogram them into iPSCs and further differentiate them into retinal organoids. The bright-field microscopy and hematoxylin and eosin (H&E) staining data confirmed that, in contrast to the retinal organoids from control subjects, the XLRS patient-specific iPSC-3D-retinal organoids exhibited the XLRS-specific splitting phenotype after 150 days of differentiation (FIGS. 3A-3B), consistent with our previous observations [10a]. Sanger sequencing confirmed the specific Rs1 point mutation and the alteration in amino acids of patient-specific iPSCs, identical to that found in the respective patient (FIGS. 3C-3D). Next, we generated an XLRS knock-in mouse model harboring the same patient-specific p.R209C mutations via CRISPR/Cas9 technology (FIG. 2C). Finally, mice with a knock-in patient-specific RS1 mutation were generated, and Sanger sequencing was performed to confirm the change in the DNA codon CGA (arginine) to TGT (cysteine) in exon 6 of the Rs1 gene (FIG. 2D; FIG. 3H) in the knock-in XLRS mouse model (abbreviated as Rs1emR209C). The disorganization of retinal structures and impaired retinal electrophysiology were subsequently verified by OCT imaging and electroretinogram (ERG), respectively. The chronological changes in the structural integrity of the retinas of Rs1emR209C mice were evaluated via OCT imaging at 3 weeks, 6 months, and 12 months (FIG. 2E). Remarkably, severe retinoschisis was initially observed in the INL at 3 weeks of age, and the size of the retinal splitting cavities increased at 6 months of age in Rs1emR209C mice (FIG. 2E). To examine the lesion foci of retinoschisis in the ONL and INL of Rs1emR209C mice, we performed H&E staining at 3 weeks, 6 months, and 12 months in the wild-type and Rs1emR209C mouse retinas (FIG. 2F). Histological assessment revealed disorganization of the ONL and INL at 3 weeks of age and retinal splitting cavities at 6 months of age in Rs1emR209C mice (FIG. 2F). At 12 months, the aforementioned schisis-like structures were resolved, the outer plexiform layer (OPL) was diminished, and the ONL, INL, outer segment (OS), and inner segment (IS) were shortened in Rs1emR209C mouse retinas compared to those in wild-type mouse retinas (FIGS. 2E and 2F). In contrast, there were no schisis-like lesions or other significant changes in the wild-type mice at any given age (FIGS. 2E and 2F).

We also conducted various experiments to characterize the disease features and eye fundus manifestations of Rs1emR209C mice at the molecular level. First, using immunofluorescence staining with DAPI to stain cell nuclei, we counted the cell numbers in the ONL of wild-type and Rs1emR209C mouse retinas at different ages (FIG. 3I-3J). The cell density in the ONL was lower in the Rs1emR209C mouse retinas at 3 weeks (58.4 versus 72.3 cells/1000 μm2), 6 months (44.6 versus 63.7 cells/1000 μm2) and 12 months (59 versus 69.4 cells/1000 μm2) than in the wild-type retinas at the corresponding ages (FIG. 3I-3J). Protein kinase C-α (PKCA)-positive bipolar cells and DLG4-labeled postsynapses in the OPL confirmed intact neurosensory structures in wild-type retinas, while Rs1emR209C retinas exhibited reduced or absent DLG4 expression, indicating disrupted neurosensory networks in the OPL (FIG. 2G). The reduction in photoreceptor numbers and disrupted OPL neurosensory networks were consistent with the findings in the XLRS mouse model described previously [2b, 3b]. We also assessed the number of Müller glia in Rs1emR209C and control retinas by staining with the Müller glia marker, GFAP. The number of GFAP+Müller glia in the wild-type mouse retinas was similar at all tested time points, and compared with those in the age-matched control retinas, the area of Müller glia in the Rs1emR209C mouse retinas was higher (3 weeks: 2.6 versus 8.8%; 6 months: 1.8 versus 10.0%; 12 months: 2.2 versus 10.3%; FIG. 2H and FIG. 3K). Furthermore, we assessed visual function in wild-type and Rs1emR209C mice using ERG. The electrophysiological responses of the Rs1emR209C mice revealed an decrease in the amplitude of the scotopic a-wave at 3 weeks, complete absence at 6 months and 12 months (FIG. 2I and FIG. 3L), and the disappearance of dark-adapted b-wave responses at all time points compared to those of age-matched controls (FIG. 2I and FIG. 3M). In addition, the Rs1emR209C mice exhibited longer a-wave implicit time at 12 months (FIG. 3N). Altered ERG responses in Rs1emR209C mice supported retinal anatomical abnormalities, highlighting the dysfunctional synaptic transmission between photoreceptors and bipolar cells in XLRS mice. Overall, the Rs1emR209C mice and patient-specific iPSC-derived 3D-retinal organoids recapitulated the retinoschisis-like phenotype of XLRS, and the multiple ophthalmic/eye fundus manifestations of Rs1emR209C mice presented the similar clinical features with disease progression in human XLRS patients.

ScRNA-Seq Profiling of Patient-Derived Retinal Organoids and Retinas from Genetically Engineered Rs1emR209C Mice

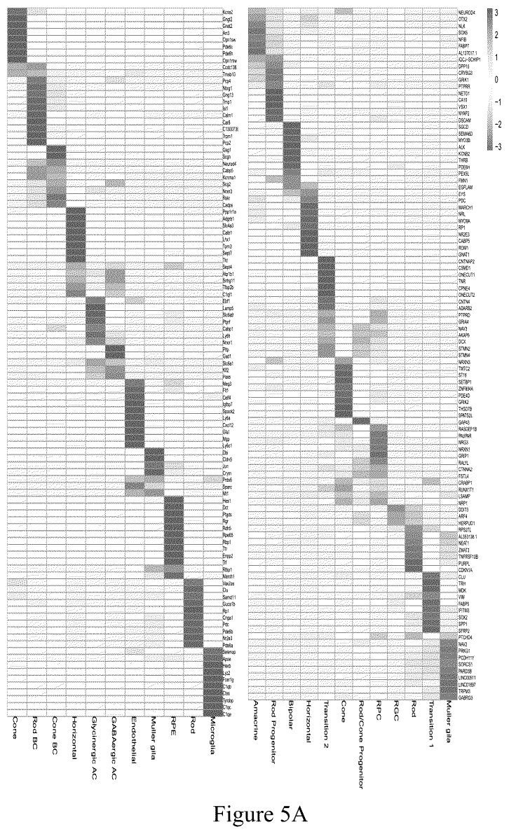

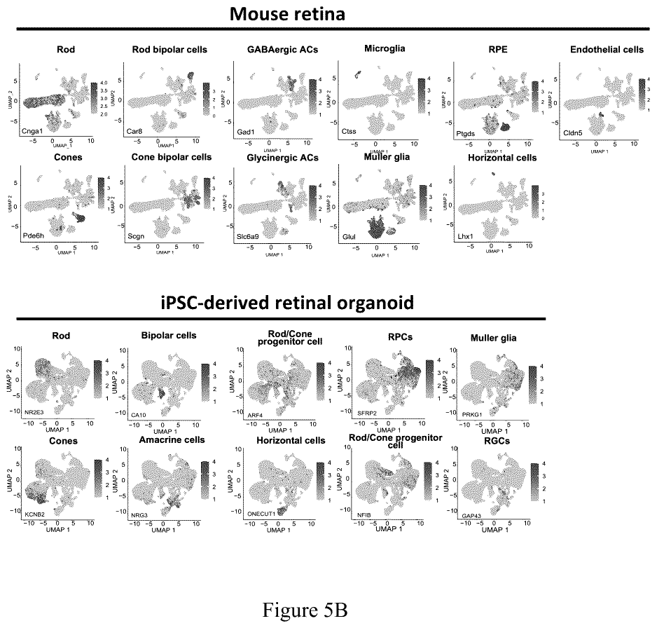

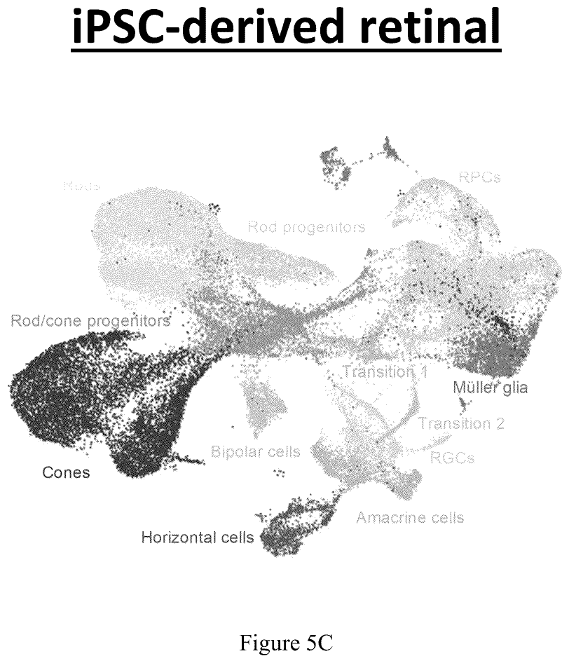

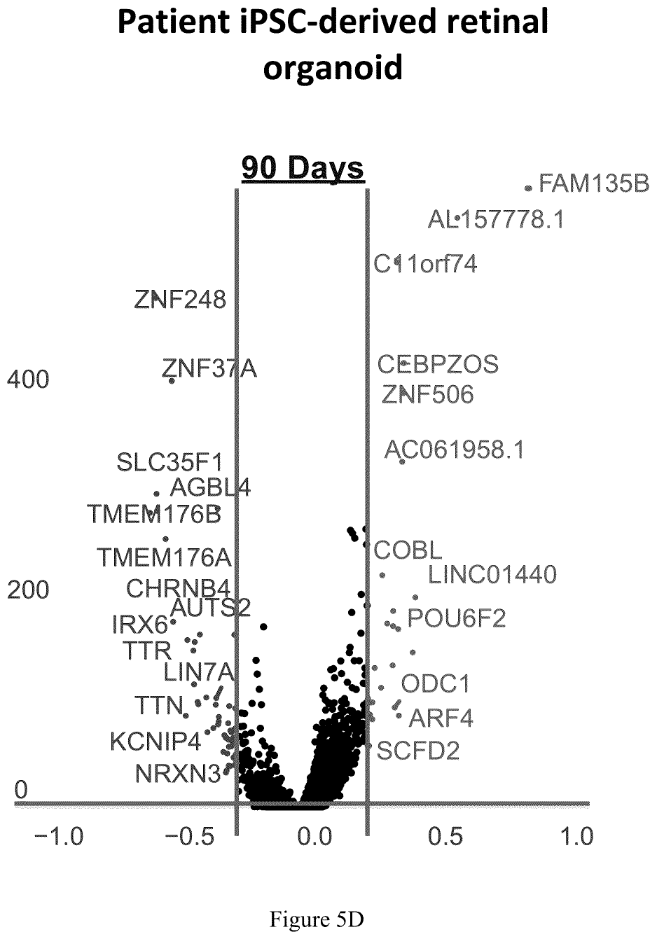

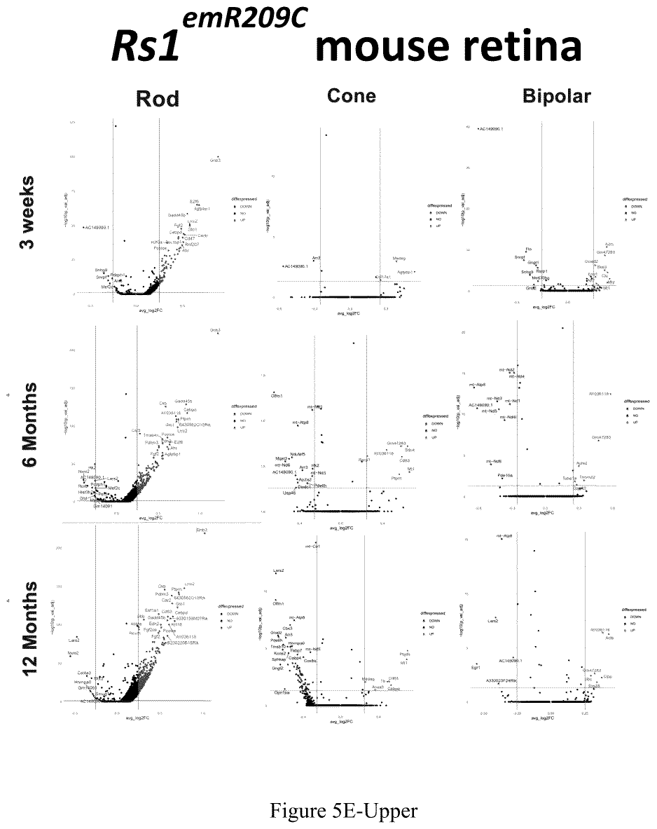

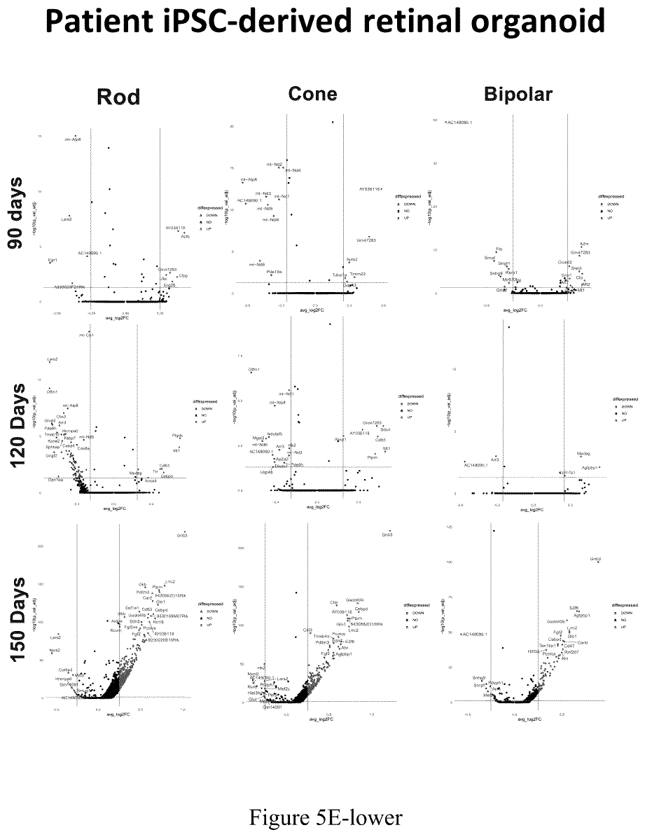

To delineate critical disease-related pathways involved in XLRS pathologies, we proposed a multimodal spatiotemporal platform that applies scRNA-seq and ST on patient-derived retinal organoids and Rs1emR209C mice (FIG. 4A). We collected both patient-derived retinal organoids and Rs1emR209C mice and subjected them to high throughput scRNA-seq, and identified critical disease-related pathways in the overlap of the transcriptomic data from both models and subsequently validated the scRNA-seq findings using ST (FIGS. 4A and 4B). Retinoschisis is predominantly observed in the INL of clinical XLRS patients [24] and RS1 is expressed in bipolar cells and photoreceptors in the retina [25]. Thus, we performed immunofluorescence staining to verify the expression and localization of the RS1 protein and the integrity of bipolar cells in wildtype and Rs1emR209C retinas prior to scRNA-seq. In wild-type mice, the RS1 protein was predominantly localized to the IS, OPL, and INL, in which OPL and IPL were positively stained by the well-known bipolar cell marker PKCA (FIG. 4C). In contrast, the Rs1emR209C retina exhibited a significant reduction in RS1 expression in bipolar cells and photoreceptors, along with disorganization of PKCA-expressing bipolar cells (FIG. 4C). These data validated the localization of RS1 protein and its disease-related expression pattern in Rs1emR209C mice. In scRNA-seq, uniform manifold approximation and projection (UMAP) plots were generated for visualization. By analyzing retinas from Rs1emR209C and wild-type mice, we identified 11 different clustered cell types, including rod bipolar cells, cone bipolar cells, cones, rods, and Müller glia, among a total of 37,342 cells and 21,014 genes (FIG. 4D; FIG. 5A, left; Supplementary FIG. 4B, upper). For the retinal organoids derived from patient and control iPSCs, 10 different cell types, including rods, cones, and bipolar cells, were classified among a total of 42,293 cells and 30,579 genes (FIG. 5C, FIG. 5A, right; FIG. 5B, Lower). Leveraging the advantages of Rs1emR209C mouse retinas with naïve 3D tissue architecture and patient-specific mutations, we delved into the clusters between Rs1emR209C retinas and wild-type retinas at all tested time points to identify the vulnerable retinal cell types in XLRS (FIG. 4E). Across all groups, the total cell count of each group was equivalent. In wild-type retinas, rod cells displayed a modest increase after 3 weeks of age and remained stable from 6 months onward. Müller glia, on the other hand, remained relatively unaffected across all ages in wild-type mice. With the Rs1emR209C mice ages, there was a noticeable decreasing trend in the proportions of rod and cone cells, while the proportion of Müller glia gradually increased (FIG. 4E). Consistent with the observations in FIG. 3I-3J, the scRNA-seq-based approach also indicated a reduction in photoreceptors and an increase in Müller glia in Rs1emR209C retinas.

Next, we performed a deep scRNA-seq analysis of bipolar cells and photoreceptors to dissect the XLRS pathologies and disease-related genes in both Rs1emR209C mice and XLRS patient derived retinal organoids. Along with the clustering of specific retinal cell types using scRNA-seq from Rs1emR209C mouse retinas and patient-derived retinal organoids, we identified down- and upregulated differentially expressed genes (DEGs) (avg log 2FC>0.25, p-value adj<0.05) in the Rs1emR209C retina and the patient-specific retinal organoid bipolar cells at different time points (FIG. 4F; FIG. 5D-5E). To elucidate the pathways influenced by the mutation of Rs1 in both in vivo and in vitro models, we performed the Ingenuity Pathway Analysis (IPA) of the DEGs in bipolar cells and photoreceptors at different time points (FIG. 4G; FIG. 5F). The IPA results revealed that endoplasmic reticulum (ER) stress related signaling pathways, including eukaryotic initiation factor 2 (eIF2) signaling, the protein ubiquitination pathway, and the unfolded protein response pathway, were consistently enriched in the bipolar cells, cones, and rods from the Rs1emR209C retinas and XLRS patient-specific retinal organoids at all tested time points (FIG. 4G; FIG. 5F). To further identify the pathways that are crucial and highly conserved in bipolar cells and photoreceptors between the mouse model and patient-derived organoids, we found several shared pathways between the two aforementioned experimental platforms in bipolar cells, rods, and cone cells (21 pathways in bipolar cells, 16 pathways in cones, 17 pathways in rods; FIG. 4H). The overlapped pathways among in vivo and in vitro XLRS models of bipolar cells, and cone and rod cells were listed in FIG. 4I, and further overlapping of listed pathways was conducted to identify the most vulnerable pathways to Rs1 mutation across three cell types (FIG. 4J). The results demonstrated that eIF2 signaling was the most enriched pathway in Rs1-expressing cell types from the two aforementioned XLRS-like models. In addition to eIF2 signaling, mTOR signaling, and the regulation of the eIF4 and p70S6K signaling pathways were also significantly enriched in Rs1emR209C retinas and patient-derived retinal organoids (FIG. 4J). Gene interaction analysis further revealed the close interaction network among the genes involved in eIF2 signaling, mTOR and the regulation of the eIF4 and p70S6K signaling pathways (FIG. 4K). Collectively, we combined the transcriptomic information from both models and found eIF2 signaling and other concomitant partners as highly enriched and conserved pathways in Rs1-expressing cells from Rs1emR209C mouse retinas and XLRS patient iPSC-derived 3D retinal organoids.

In Situ Spatiotemporal Transcriptomics of Rs1emR209C Retinas Validated the Enrichment of Disease-Related Pathways Identified by scRNA-Seq

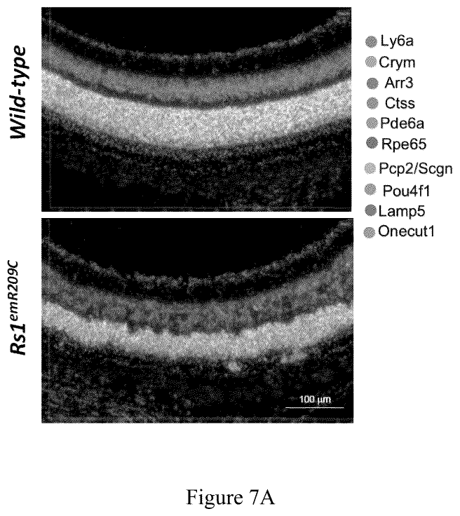

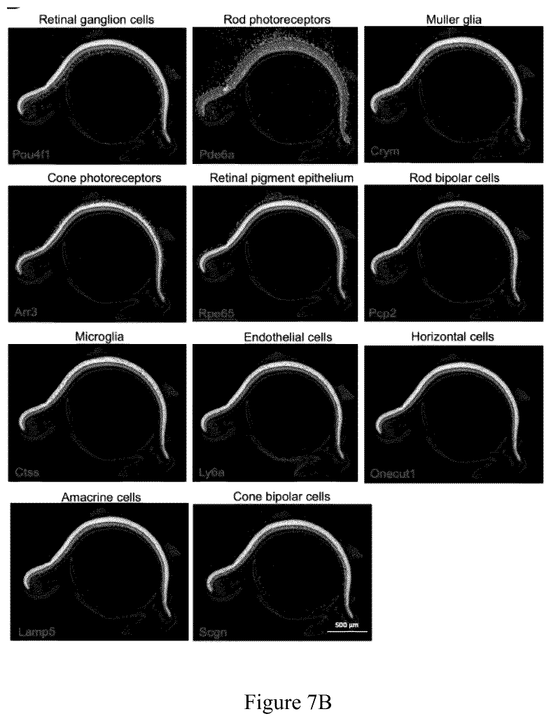

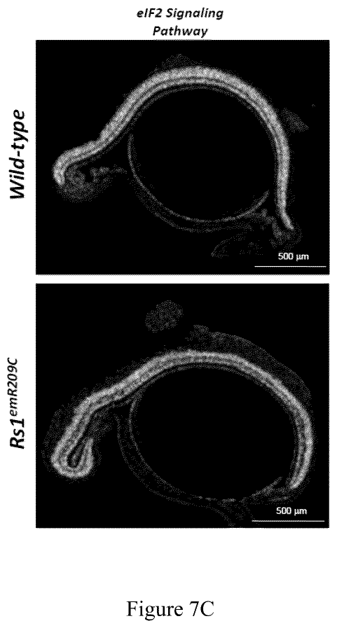

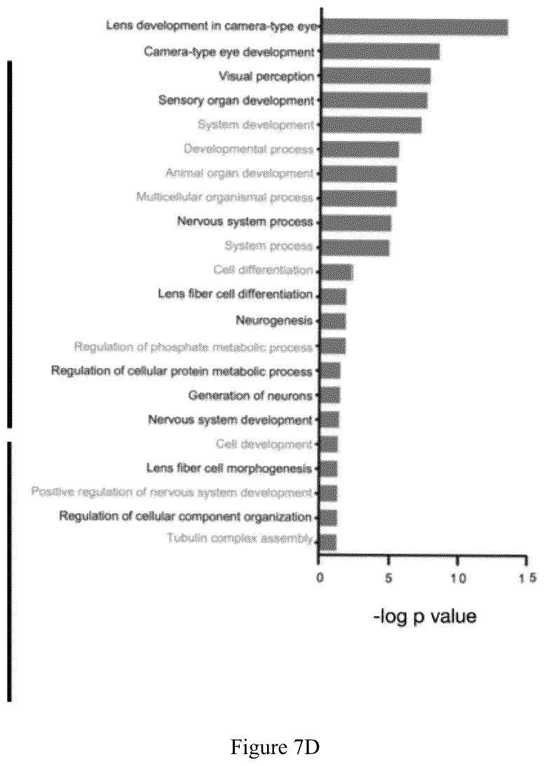

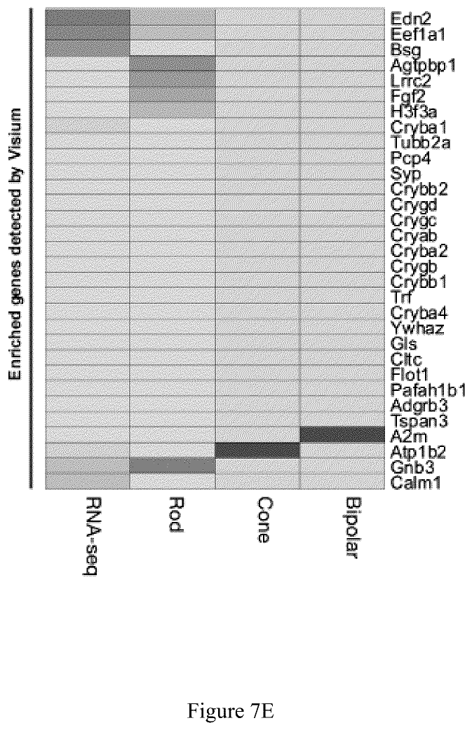

Xenium, a novel in situ ST platform, is capable of capturing mRNA within cells and employs spatial labeling methods to project genetic data onto H&E-stained or confocal images. Following the Xenium analysis, we first conducted marker gene analysis to visualize known cell markers and identify corresponding cells. We identified retinal ganglion cells, rod and cone photoreceptors, retinal pigment epithelium, microglia, and endothelial cells by staining for each specific marker, including Ly6a, Crym, Arr3, Ctss, Pde6a, Rpe65, Pep2 Scgn, Pou4f1, Lamp5, and Onecut1 (FIG. 6A; Upper left inset: H&E-stained sections, the rest: Detection of gene expression using Xenium; FIGS. 7A and 7B). Given that our findings identified eIF2 signaling as the most enriched disease-related pathway in the overlapping transcriptomic signatures from Rs1emR209C retinas and patient-derived retinal organoids, we then focused on analyzing the eIF2 signaling pathway after completing a layered analysis of the retinal cells (FIG. 6B; FIG. 7C). In addition to assessing eIF2 signaling, we simultaneously assessed other enriched pathways, such as the mTOR pathway and the regulation of eIF4 and p70S6K pathways (FIGS. 6C and 6D), which were previously identified through scRNA-seq in bipolar, rod, and cone cells (FIG. 4). Consistent with our scRNA-seq findings (FIG. 4), the spatial mapping of selected genes indicated enrichment of eIF2 signaling, mTOR, and the regulation of eIF4 and p70S6K pathways at all given ages during the 1-year observation in Rs1emR209C mouse retinas (FIGS. 6B, 6C and 6D).

In addition to Xenium, we applied another ST platform, Visium CytAssist, to spatially map gene expression in 3-week-old wild-type and Rs1emR209C mouse retinas (FIG. 6E). The location of the captured spots matched that of the H&E-stained wild-type and mutant retinas, and these spots were further classified into 4 retinal layers, namely, the retinal ganglion cell layer, the inner cell layer, the outer cell layer, and the retinal pigment epithelium (FIG. 6E). The functional analysis of the shared DEGs in the ST, scRNA-seq, and bulk RNA-seq data indicated developmental abnormalities in the mutant retinas (FIG. 7D). In addition, among 34 enriched genes recognized in Visium CytAssist, (nb3 and Edn2 were also up-regulated in the bulk RNA-seq and scRNA-seq of rod cells (FIG. 6F, FIG. 7E). (nb3 and Edn2 are associated with the abnormalities in retinal phototransduction [26] and gliosis [27], respectively. In both Visium CytAssist and scRNA-seq, remarkable enrichment of Edn2 and Gnb3 was observed in the Rs1emR209C retinas compared to the wild-type retinas (FIGS. 6G and 6H). As shown by scRNA-seq, Gnb3 and Edn2 were particularly enriched predominantly in the rod cells and Müller glia of Rs1emR209C retinas (FIGS. 6G and 6H, lower). Collectively, our ST data provided spatiotemporal transcriptomic evidence that eIF2 signaling, the mTOR pathway, and the regulation of eIF4 and p70S6K pathways were all chronically enriched in XLRS pathogenesis, validating the crucial disease-related pathways identified by scRNA-seq.

### Validation of the Enrichment of Endoplasmic Reticulum Stress/eIF2α Pathway Pathways in Rs1EmR209C Retinas

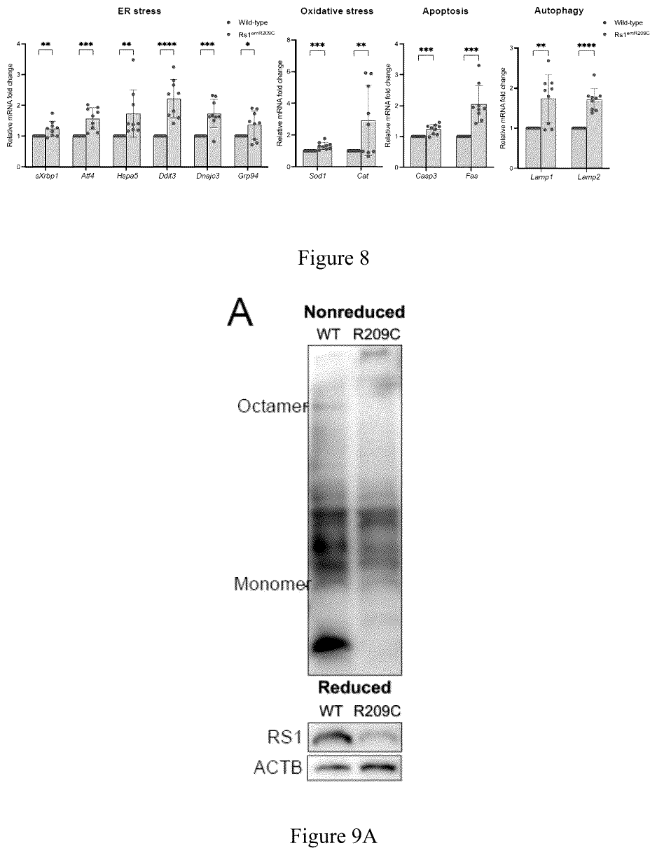

Endoplasmic reticulum (ER) stress is well recognized for triggering the activation of unfolded protein response (UPR) pathways. This process begins when glucose-regulated proteins (GRPs) dissociate from three key ER transmembrane sensors: Inositol-Requiring Protein 1 (IRE1), Activating Transcription Factor 6 (ATF6), and Protein Kinase R-like ER Kinase (PERK). Within the UPR signaling cascades, PERK plays a crucial role by phosphorylating eIF2α, which subsequently initiates downstream signaling to inhibit protein synthesis. Meanwhile, IRE1 and ATF6 contribute to protein homeostasis by enhancing protein folding and degrading misfolded proteins, respectively [28]. Under normal conditions, phosphorylated PERK-induced eIF2α phosphorylation leads to a protective response by globally reducing protein production and selectively promoting the translation of Activating Transcription Factor 4 (ATF4) [29]. However, when ER stress persists, this adaptive UPR response is overwhelmed, causing phosphorylated PERK to switch to a pro-apoptotic pathway by activating CHOP, ultimately resulting in cell death [30]. Along with the observations of the most enriched eIF2 pathways identified by scRNA-seq and ST in Rs1emR209C retinas, we next validated the possible involvement of these critical disease related pathways and of other noxious cellular events in XLRS pathologies. We performed quantitative real-time PCR (qRT-PCR) to examine and compare the expression of indicated genes between Rs1emR209C retinas and wild-type retinas. Overall, we verified the upregulation of genes associated with conserved ER stress pathway (sXbp1 and Atf4) and ER stress target genes (Hspa5, Ddit3, Dnajc3, and Grp94) in Rs1emR209C retinas compared with wild-type retinas (FIG. 8A). Meanwhile, we also observed the upregulation of apoptosis-related genes (Casp3 and Fas), oxidative stress-related genes (Sod1 and (at), and autophagy-related genes (Lamp1 and Lamp2) in the Rs1emR209C retinas (FIG. 8A).

Mutations in RS1 can disrupt its octamer structure and produce misfolded RS1 protein, which hinders its transport to the cell surface [8]. To verify whether the patient-specific RS1 mutation also induces RS1 misfolding in Rs1emR209C mouse retinas, we first examined whether the Rs1 knock-in R209C mutation affects the formation of the RS1 protein homo-octameric complex. We analyzed the lysates from the retinas of both wild-type and Rs1emR209C mice using nonreducing SDS polyacrylamide gradient gels (FIG. 9A upper). As shown in the nonreduced gels, the wildtype RS1 octamer migrated to an expected size of approximately 180 kDa. In contrast, RS1 from Rs1emR209C mice failed to produce a mature complex despite the expression of the monomer (24 kDa; FIG. 9A upper). In the reduced gels, total RS1 protein was largely decreased in mutant retinas compared to wild-type retinas (FIG. 9A lower, and FIG. 9B). These data indicated that the patient-specific R209C mutation impaired protein folding and the formation of the homooctameric complex, consecutively contributing to reduced RS1 protein expression. Impaired protein folding and increased ER stress can induce the accumulation of BiP and the activation of UPRs [31]. Among the three branches of UPR, the eIF2α pathway acutely reduced protein translation. Its downstream effector, ATF4, stimulates the expression of proteins involved in cell recovery and adaptation [30]. Under prolonged ER stress, phosphorylated eIF2α further promotes the activation of ATF4 and CHOP, leading to ER stress-induced apoptosis [28, 30]. Accordingly, we next examined if the patient-specific p.R209C mutation leads to prolonged ER stress in Rs1emR209C mice. Along with the observations of impaired RS1 protein folding, Western blot analysis revealed a robust increase in the protein levels of BiP (FIGS. 9C and 9F) and concomitant phosphorylation of eIF2α (FIGS. 9D and 9G). The protein levels of ATF4 and CHOP were also increased in Rs1emR209C retinas compared with wild-type retinas (FIGS. 9E, 9H, and 9I). These data indicated that long-term failure of RS1 protein folding and assembly contributed to prolonged ER stress and upregulated eIF2α signaling. To examine whether this upregulation of eIF2α signaling affects protein translation, we used a protein synthesis assay to incorporate puromycin into nascent proteins and measure newly synthesized proteins as described previously [32]. As detected by a puromycinylated protein antibody, newly synthesized proteins over a wide range of molecular weights were present in the control retinas. However, protein synthesis was barely detectable in the Rs1emR209C retinas (FIGS. 9J and 9K). Given that CHOP is the effector protein of ER stress-induced apoptosis, we used a TUNEL assay to evaluate apoptosis in the Rs1emR209C retinas. Apoptotic signals were detected in the disorganized ONL in age-matched Rs1emR209C retinas (FIG. 9L). Furthermore, we used IF staining to identify the ER stress-related proteins at the lesion sites in Rs1emR209C retinas and compared the expression of these proteins to wild-type retinas. In the wild-type retinas, no phosphorylated PERK (pPERK) was detected at any given age (FIG. 9M, left). Compared to age-matched wild-type retinas, Rs1emR209C retinas showed no detectable PERK phosphorylation at 3 weeks of age but exhibited a time-dependent increase in PERK phosphorylation in the IS and OS, where photoreceptors are located, at 6 months and 12 months (FIG. 9M, right). Furthermore, we investigated the impact of the Rs1 knock-in p.R209C mutation on proteomic changes in Rs1emR209C retinas using liquid chromatography-mass spectrometry (LC/MS)-based proteomic analysis. At 3 weeks of age, Rs1emR209C retinas exhibited a significant reduction In overall protein expression compared to age-matched wild-type retinas (FIG. 9N). The differences in protein expression between Rs1emR209C and wild-type retinas were further analyzed. Notably, RS1 protein levels were significantly reduced in Rs1emR209C retinas, while ER molecular chaperones BiP and glucose-related protein 94 (GRP94) were upregulated (FIGS. 90 and 9P). In addition to the findings of RS1, BiP, and GRP94, other enriched proteins included p38a (downstream target of the IRE1 UPR branch), GFAP (the Müller glia marker), DNAJC3 (a chaperone of UPR), superoxide dismutase type 1 (SOD1) and catalase (antioxidant enzymes), and Caspase-3 (a key mediator of apoptosis) (FIG. 9O and FIG. 9P). These findings indicate the activation of the UPR and the impaired production of RS1 in Rs1emR209C retinas. Together, the Rs1emR209C mice exhibited XLRS phenotypes accompanied by failure of RS1 octamer structure formation, prolonged accumulation of ER stress and the enrichment of eIF2α pathway, impaired protein synthesis, and increased ER stress-induced apoptosis (FIG. 9Q).

### Therapeutic Targeting of Chronic ER Stress/eIF2α Pathway Activation Ameliorated XLRS Associated Structural and Functional Abnormalities

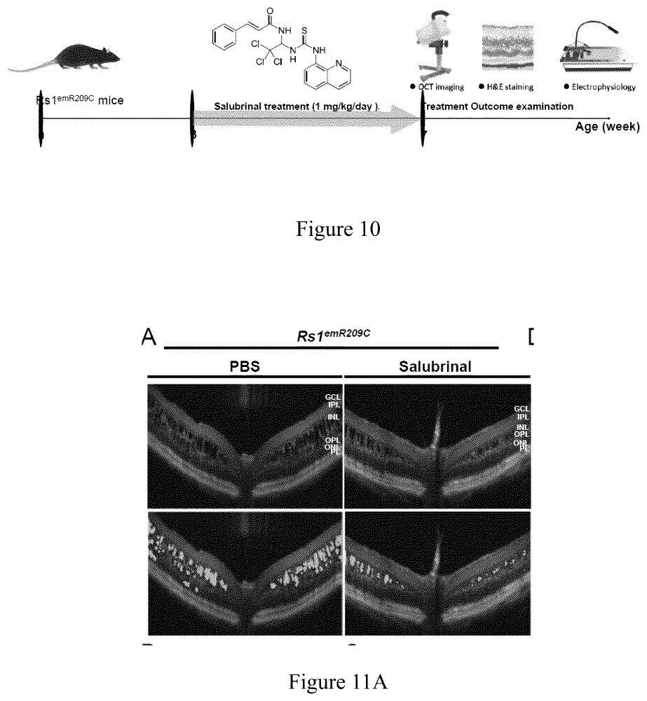

Salubrinal has been identified as a selective inhibitor of the GADD34/protein phosphatase 1 complex, which is responsible for the dephosphorylation of eIF2α [22]. This small molecule exhibits potent efficacy in suppressing apoptosis induced by ER stress, while it does not affect apoptosis unrelated to ER stress [22]. The cytoprotective effects of salubrinal are widely attributed to its ability to prolong eIF2α phosphorylation, which in turn inhibits global protein translation, reduces the ER's workload, and downregulates ER stress markers [23]. Furthermore, some studies also reported that salubrinal might also influence the IRE1/p38-dependent pathway [33], a distinct UPR branch, leading to a reduction in downstream apoptotic cell death. This effect might be associated with the overall decrease in ER stress linked to prolonged eIF2α phosphorylation, though this remains speculative and is not yet fully understood [23]. To test the contribution of dysregulated ER stress and the chronically enriched eIF2α pathway to the XLRS-like phenotypes in Rs1emR209C retinas, we chronically treated mutant mice with salubrinal to inhibit eIF2α phosphatase, which led to constitutive activation of eIF2 pathway signaling and the mitigation of ER unfolded protein load in Rs1emR209C mice. Either salubrinal (1 mg/kg/day) or PBS was intraperitoneally administered to 3-week-old Rs1emR209C mice for 28 consecutive days, after which the treated mice were subjected to phenotypic assessments (FIG. 10A). After a 28-day salubrinal treatment, OCT images showed the reduced area of retinal splitting cavities (FIG. 11A and FIG. 11B) and increased ONL thickness (FIG. 11C) in the salubrinal-treated Rs1emR209C retinas compared to the PBS-treated Rs1emR209C retinas. To verify the treatment efficacy of salubrinal, we further performed H&E staining to assess retinoschisis lesions in the ONL and INL retinal layers of Rs1emR209C retinas treated with either PBS or salubrinal. After salubrinal treatment, the stained images revealed a significant reduction in the number of retinal splitting cavities in the retinas of salubrinal-treated Rs1emR209C mice compared with the retinas of the PBS-treated Rs1emR209C mice (FIG. 11D and FIG. 11E). An increase in ONL thickness was also observed in Rs1emR209C retinas via H&E staining (FIG. 11F). Next, we recorded the dark-adapted ERG responses to light stimuli at various intensities to evaluate the electrophysiological function of the Rs1emR209C retinas (FIG. 11G). Compared to the PBS-treated Rs1emR209C mouse, the light response curves of scotopic a-wave and b-wave were upward-shifted by the administration of salubrinal (FIG. 11H and FIG. 11I). The maximal a-wave amplitudes in the salubrinal-treated retinas were achieved under light stimulation at 1 log cd·s/m2 (salubrinal-treated retinas vs. PBS-treated retinas=80.86 vs. 44.70 μV; FIG. 11G and FIG. 11H). The b-wave amplitude elicited by light stimulation at 1 log cd·s/m2 was 91.41 μV (salubrinal-treated retinas vs. PBS-treated retinas=91.41 vs. 49.71 μV; FIG. 11G and FIG. 11I). The a-wave implicit time was significantly shortened by light stimulation at 0 log cd·s/m2 in the salubrinal-treated retinas than in the PBS-treated retinas of Rs1emR209C mice (salubrinal-treated retinas vs. PBS-treated retinas=17.88 ms vs. 24.44 ms; FIG. 11G and FIG. 11J). In addition to the OCT imaging and electroretinogram findings, we employed immunofluorescence to evaluate the efficacy of salubrinal on the integrity of the neurosensory network in the disorganized retinas of Rs1emR209C mice. Compared to PBS-treated Rs1emR209C retinas, salubrinal treatment significantly increased the presence of PKCA-stained bipolar cells in the IPL, INL, and OPL layers (FIG. 11K and FIG. 11N). Similarly, DLG4-labeled structures were notably enhanced in the OPL layers following salubrinal treatment (FIG. 11L and FIG. 11O). These results indicate that salubrinal rescued bipolar cells and restored the postsynaptic density of neurons in the OPL. Moreover, the enrichment of recoverin in the ONL layer suggested that salubrinal treatment mitigated photoreceptor loss in Rs1emR209C retinas (FIG. 11M and FIG. 11P). Collectively, these immunofluorescence findings demonstrate that salubrinal improved bipolar cell integrity, photoreceptor survival, and postsynaptic transmission from photoreceptors to bipolar cells. This data highlights the potential of targeting ER stress-associated apoptosis to ameliorate the severity and progression of XLRS in Rs1emR209C retinas, supporting the role of chronic eIF2 pathway activation in the disruption of retinal architecture, progressive retinoschisis, and impaired visual function in the XLRS mouse model.

### In Situ Spatiotemporal Transcriptomics Evidence Verifies the Diminishment of Disease-Related Pathways in Salubrinal-Treated Rs1emR209C Mice

To examine the post-treatment outcome on the crucial disease pathways identified by the multimodal transcriptomic approach, we additionally performed Xenium ST to assess the spatial expression profiles of crucial disease pathways in Rs1emR209C mice after therapeutic targeting. Using marker gene analysis, the cell markers were visualized to identify the corresponding retinal cell types in Rs1emR209C mouse retina receiving the administration of salubrinal or PBS. Indicated retinal cell types were shown via staining for specific markers (FIG. 12A). Along with the observations in FIGS. 3, FIG. 6, FIG. 9 and FIG. 11, we focused on the spatiotemporal mapping of DEGs corresponding to eIF2 signaling, mTOR, and the regulation of eIF4 and p70S6K pathways (FIGS. 12B, 12C, and 12D). Among the retina samples from 3-week-old Rs1emR209C mice treated with salubrinal or PBS, we distinguished and analyzed the genes related to eIF2 signaling pathway, mTOR pathway, and the regulation of eIF4 and p70S6K pathways and quantified the signals to compare the differential expression of these genes between treated and untreated mice (FIGS. 12B, 12C, and 12D). Compared to PBS treatment, salubrinal treatment effectively suppressed the enrichment of the genes related to these pathways in the ONL layer. Meanwhile, we also verified the changes in Gnb3 and Fdn2 at the transcript and protein levels using Xenium analysis and immunofluorescence (FIGS. 12E-12H). Consistent with the findings of Visium CytAssist and scRNA-seq, the Xenium results also revealed the upregulation of Gnb3 and Edn2, which were predominantly localized at the ONL of s/emR209C retinas at 3 weeks of age (FIGS. 12E, 12F, 12I and 12J). The protein levels of Gnb3 and Edn2 were also increased in the ONL layer (FIGS. 12E, 12F, 12I and 12J). We also evaluated and compared the expression of Gnb3 and Edn2 in Rs1emR209C retinas treated with salubrinal or PBS (FIGS. 12G, 12H, 12K, and 12L). Xenium and IF data indicated that salubrinal treatment effectively abrogated the upregulation of Gnb3 and Edn2 at both the transcriptional and protein levels (right in FIGS. 12G, 12H, 12K, and 12L). The diminishment of Gnb3 and Edn2 by salubrinal treatment suggested the alleviation of retinal gliosis and abnormal phototransduction. Overall, these data provided spatiotemporal transcriptomic evidence revealing the therapeutic outcome on the crucial disease pathways identified by the multimodal transcriptomic platforms.

### Therapeutic Targeting of Chronic ER Stress/eIF2α Pathway Activation Enhanced the Efficacy of AAV-Mediated RS1 Gene Delivery

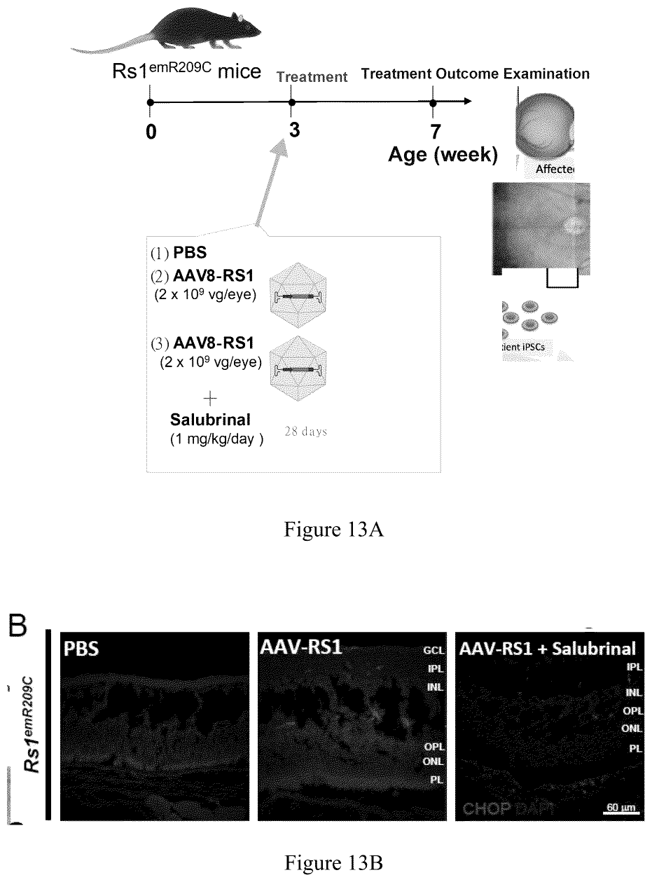

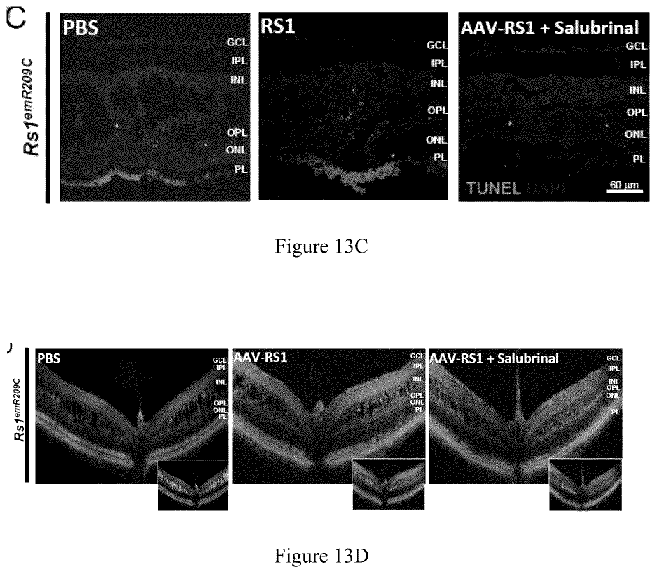

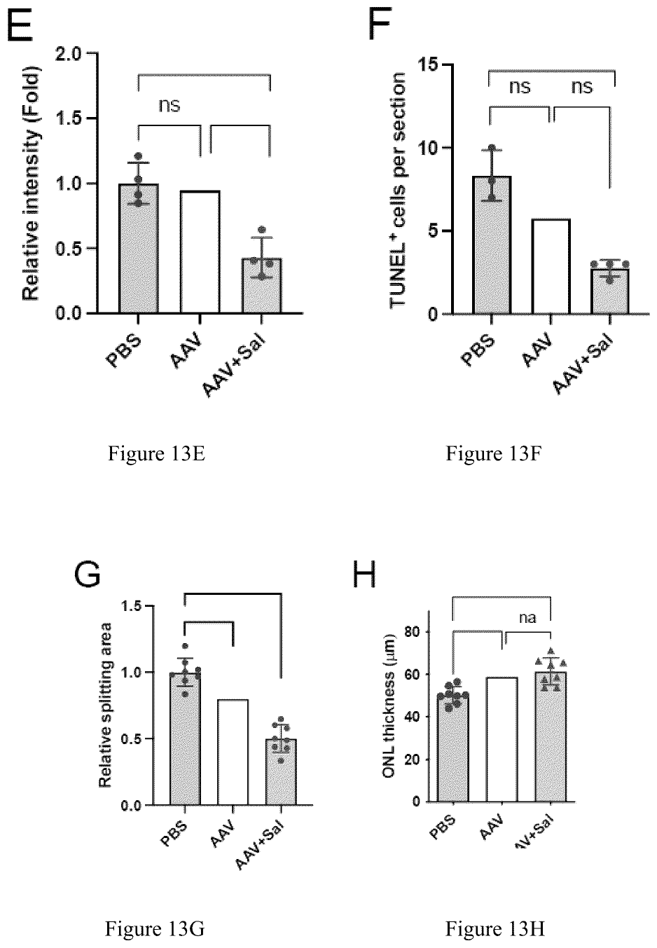

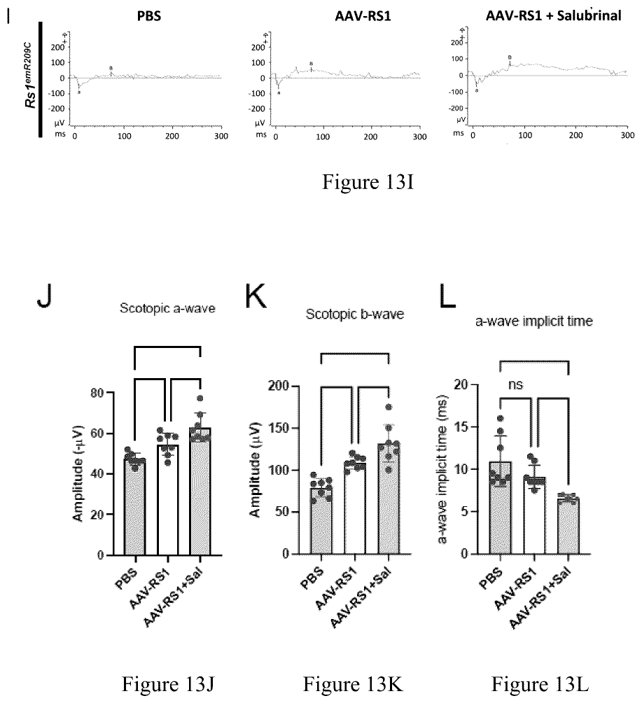

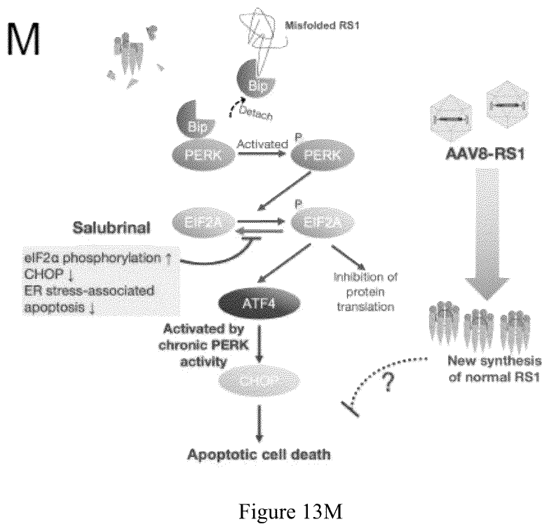

Several studies have reported the impressive efficacy of AAV-based RS1 gene delivery in XLRS mouse models [4a, 4b, 34]. These studies primarily utilized Rs1-knockout mice, and the AAV-based RS1 gene delivery typically exhibited excellent efficacy after approximately 4 to 6 months [2b, 4b, 35]. In a subsequent clinical trial, AAV-mediated RS1 gene delivery generally produced unsatisfactory outcomes, only transiently closing retinoschisis cavities in one out of nine participants after 18 months [5]. The exact mechanisms behind the inconsistent efficacy of RS1 gene delivery remain unclear, likely due to some auxiliary disease mechanisms stemming from RS1 gene mutations. To investigate whether the therapeutic targeting of the chronic eIF2α signaling activation could enhance the efficacy of AAV-based RS1 gene delivery, we intravitreally injected the human RS1 gene through the AAV delivery system (2×109 vg/eye) into 3-week-old Rs1emR209C mice and intraperitoneally injected either salubrinal (1 mg/kg/day) or PBS for 28 consecutive days. Phenotypic examination was also conducted after the 28-day course of the experiment (FIG. 13A). Considering that CHOP is an effector of the eIF2 signaling pathway that contributes to ER stress-induced apoptosis, we used IF and TUNEL assays to compare CHOP expression and changes in apoptotic signals in Rs1emR209C retinas with indicated treatments, respectively. AAV8-mediated RS1 gene delivery did not modify CHOP expression, whereas the combination of AAV8-mediated gene delivery plus salubrinal modestly attenuated CHOP expression (FIGS. 13B and 13E). TUNEL-positive apoptotic signals were consistently observed throughout the disorganized ONL in Rs1emR209C retinas. AAV8-RS1 treatment for 28 days showed a trend in reducing apoptotic cells but did not reach statistical significance (FIGS. 13C and 13F). Notably, salubrinal treatment plus AAV8-RS1 gene delivery effectively prevented the generation of apoptotic signals (FIGS. 13C and 13F).

As detected by OCT imaging, Rs1emR209C mice consistently exhibited severe retinal splitting at 3 weeks of age (FIG. 13D). Compared to those in retinas from PBS-treated Rs1emR209C mice, AAV8-based RS1 gene delivery led to a moderate reduction in retinal cavities, and the administration of salubrinal additionally decreased the cavity areas (FIGS. 13D and 13G). Similarly, an increase in ONL thickness was observed in AAV8-RS1-treated mice combined with salubrinal administration (FIGS. 13D and 13H). Subsequently, we assessed dark-adapted ERG responses to light stimuli at various intensities in Rs1emR209C mice subjected to the indicated treatments (FIGS. 13I-13L). Compared to those of Rs1emR209C mice injected with PBS, the light response curves of the scotopic a-wave and b-wave were mildly increased by AAV8-RS1 but largely increased by the combination of AAV8-RS1 and salubrinal administration (a-wave: PBS treated retinas vs. AAV8-RS1-treated retinas=47.24 vs. 54.47 μV; b-wave: PBS-treated retinas vs. AAV8-RS1-treated retinas=78.97 vs. 108.71 μV; FIGS. 13I-13K). The b-wave amplitude elicited by light stimulation at 1 log cd·s/m2 was 131.8 μV in the AAV8-RS1-salubrinal-treated retinas. The combined treatment of AAV8-RS1 plus salubrinal exhibited a synergistic response in the elevation of b-wave amplitude (PBS-treated retinas vs. AAV8-RS1-salubrinal-treated retinas=78.97 vs. 131.8 μV; FIGS. 13I and 13K). The maximal a-wave amplitude of the retinas was achieved under light stimulation at 1 log cd·s/m2 and the combination of AAV8-RS1 plus salubrinal also elicited the synergistic responses (PBS-treated retinas vs. AAV8-RS1-salubrinal-treated retinas=47.24 μV vs. 62.675 μV; FIGS. 13I and 13J). Collectively, AAV8-RS1 moderately increased the amplitudes of the scotopic a- and b-waves, while the addition of salubrinal synergistically enhanced the efficacy of AAV8-RS1 (FIGS. 13I-13K). The a-wave implicit time after light stimulation at 0 log cd·s/m2 was significantly shorter in the AAV8-RS1-salubrinal treated retinas than in the retinas of Rs1emR209C mice administered the other two treatments (PBS treated retinas vs. AAV8-RS1-treated retinas vs. AAV8-RS1-salubrinal-treated retinas=10.93 vs. 9.125 vs. 6.56 ms; FIG. 13L). Overall, therapeutic targeting of the crucial disease pathways enhanced the efficacy of AAV-mediated RS1 gene delivery (FIG. 13M). Taken together, our data have provided evidence that the chronic activation of ER stress/eIF2α signaling are crucial pathways for XLRS pathogenesis and progression. We demonstrated that multiomic approaches integrating ST and scRNA-seq can effectively explore the complex transcriptomic signatures and pathomechanisms of XLRS both in vitro and in vivo. This platform also offers a personalized medicine-based strategy for developing novel therapeutic targets to treat incurable ophthalmic disorders (FIG. 14).

### CONCLUSION

It was found in the present invention that utilizing two disease models with patient specific gene mutations, combined with the identification of novel transcriptional signatures through multiomics approaches, can deepen our understanding of disease mechanisms and advance the development of treatment strategies for XLRS. Furthermore, future applications of a personalized medicine-based multimodal transcriptomic approach will not only enable precise decoding of the molecular pathogenesis of unexplored ophthalmic degenerations but also aid in discovering new precision-medicine-based therapeutic strategies for severe, incurable hereditary diseases like XLRS.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment. Conversely, various features that are described in the context of a single embodiment or example can also be implemented in multiple embodiments or examples separately or in any appropriate suitable sub-combination.

### REFERENCES

[1] N. D. George, J. R. Yates, A. T. Moore, Br J Ophthalmol 1995, 79 (7), 697, https://doi.org/10.1136/bjo.79.7.697.
[2] a) C. G. Sauer, A. Gehrig, R. Warneke-Wittstock, A. Marquardt, C. C. Ewing, A. Gibson, B. Lorenz, B. Jurklies, B. H. Weber, Nat Genet 1997, 17 (2), 164, https://doi.org/10.1038/ng1097-164; b) Y. Zeng, Y. Takada, S. Kjellstrom, K. Hiriyanna, A. Tanikawa, E. Wawrousek, N. Smaoui, R. Caruso, R. A. Bush, P. A. Sieving, Invest Ophthalmol Vis Sci 2004, 45 (9), 3279, https://doi.org/10.1167/iovs.04-0576.
[3] a) B. H. Weber, H. Schrewe, L. L. Molday, A. Gehrig, K. L. White, M. W. Seeliger, G. B. Jaissle, C. Friedburg, E. Tamm, R. S. Molday, Proc Natl Acad Sci USA 2002, 99 (9), 6222, https://doi.org/10.1073/pnas.092528599; b) S. Kjellstrom, R. A. Bush, Y. Zeng, Y. Takada, P. A. Sieving, Invest Ophthalmol Vis Sci 2007, 48 (8), 3837, https://doi.org/10.1167/iovs.07-0203; c) M. Liu, J. Liu, W. Wang, G. Liu, X. Jin, B. Lei, Front Med (Lausanne) 2022, 9, 886947, https://doi.org/10.3389/fmed.2022.886947.
[4] a) J. Ou, C. Vijayasarathy, L. Ziccardi, S. Chen, Y. Zeng, D. Marangoni, J. G. Pope, R. A. Bush, Z. Wu, W. Li, P. A. Sieving, J Clin Invest 2015, 125 (7), 2891, https://doi.org/10.1172/JCI81380; b) R. A. Bush, Y. Zeng, P. Colosi, S. Kjellstrom, S. Hiriyanna, C. Vijayasarathy, M. Santos, J. Li, Z. Wu, P. A. Sieving, Hum Gene Ther 2016, 27 (5), 376, https://doi.org/10.1089/hum.2015.142; c) Y. Zeng, R. S. Petralia, C. Vijayasarathy, Z. Wu, S. Hiriyanna, H. Song, Y. X. Wang, P. A. Sieving, R. A. Bush, Invest Ophthalmol Vis Sci 2016, 57 (9), OCT277, https://doi.org/10.1167/iovs. 15-18920.
[5] C. Cukras, H. E. Wiley, B. G. Jeffrey, H. N. Sen, A. Turriff, Y. Zeng, C. Vijayasarathy, D. Marangoni, L. Ziccardi, S. J. M. T. Kjellstrom, 2018, 26 (9), 2282.
[6] a) R. S. Molday, U. Kellner, B. H. Weber, Prog Retin Eye Res 2012, 31 (3), 195, https://doi.org/10.1016/j.preteyeres.2011.12.002; b) T. Wang, C. T. Waters, A. M. Rothman, T. J. Jakins, K. Romisch, D. Trump, Hum Mol Genet 2002, 11 (24), 3097, https://doi.org/10.1093/hmg/11.24.3097; c) W. W. Wu, R. S. Molday, J Biol Chem 2003, 278 (30), 28139, https://doi.org/10.1074/jbc.M302464200.
[7] M. Georgiou, L. Finocchio, K. Fujinami, Y. Fujinami-Yokokawa, G. Virgili, O. A. Mahroo, A. R. Webster, M. Michaelides, Ophthalmology 2022, 129 (5), 542, https://doi.org/10.1016/j.ophtha.2021.11.019.
[8] W. W. Wu, J. P. Wong, J. Kast, R. S. Molday, J Biol Chem 2005, 280 (11), 10721, https://doi.org/10.1074/jbc. M413117200.
[9] a) D. Chen, T. Xu, M. Tu, J. Xu, C. Zhou, L. Cheng, R. Yang, T. Yang, W. Zheng, X. He, R. Deng, X. Ge, J. Li, Z. Song, J. Zhao, F. Gu, Front Mol Neurosci 2017, 10, 453, https://doi.org/10.3389/fnmol.2017.00453; b) Y. Liu, J. Kinoshita, E. Ivanova, D. Sun, H. Li, T. Liao, J. Cao, B. A. Bell, J. M. Wang, Y. Tang, S. Brydges, N. S. Peachey, B. T. Sagdullaev, C. Romano, Hum Mol Genet 2019, 28 (18), 3072, https://doi.org/10.1093/hmg/ddz122.
[10] a) K.-C. Huang, M.-L. Wang, S.-J. Chen, J.-C. Kuo, W.-J. Wang, P. N. N. Nguyen, K. J. Wahlin, J.-F. Lu, A. A. Tran, M. Shi, Stem cell reports 2019, 13 (5), 906; b) C. Y. Lien, T. T. Chen, E. T. Tsai, Y. J. Hsiao, N. Lee, C. E. Gao, Y. P. Yang, S. J. Chen, A. A. Yarmishyn, D. K. Hwang, S. J. Chou, W. C. Chu, S. H. Chiou, Y. Chien, Cells 2023, 12 (2), https://doi.org/10.3390/cells12020211; c) C. Y. Lee, C. H. Huang, E. Rastegari, V. Rengganaten, P. C. Liu, P. H. Tsai, Y. F. Chin, J. R. Wu, S. H. Chiou, Y. C. Teng, C. W. Lee, Y. Liang, A. Y. Chen, S. C. Hsu, Y. J. Hung, J. R. Sun, C. S. Chien, Y. Chien, Int J Mol Sci 2021, 22 (18), https://doi.org/10.3390/ijms22189869; d) C. S. Chien, Y. Chien, Y. Y. Lin, P. H. Tsai, S. J. Chou, A. A. Yarmishyn, E. Rastegari, T. X. Wang, H. B. Leu, Y. P. Yang, M. L. Wang, Y. C. Jheng, H. Lai, L. J. Ching, T. I. Huo, J. Y. Cherng, C. Y. Wang, Front Cell Dev Biol 2021, 9, 634190, https://doi.org/10.3389/fcell.2021.634190.
[11] A. Fatehullah, S. H. Tan, N. Barker, Nat Cell Biol 2016, 18 (3), 246, https://doi.org/10.1038/ncb3312.
[12] a) X. Y. Tang, L. Xu, J. Wang, Y. Hong, Y. Wang, Q. Zhu, D. Wang, X. Y. Zhang, C. Y. Liu, K. H. Fang, X. Han, S. Wang, X. Wang, M. Xu, A. Bhattacharyya, X. Guo, M. Lin, Y. Liu, J Clin Invest 2021, 131 (12), https://doi.org/10.1172/JCI135763; b) W. K. Chan, R. Griffiths, D. J. Price, J. O. Mason, Mol Autism 2020, 11 (1), 58, https://doi.org/10.1186/s13229-020-00360-3.
[13] A. Strikoudis, A. Cieslak, L. Loffredo, Y. W. Chen, N. Patel, A. Saqi, D. J. Lederer, H. W. Snoeck, Cell Rep 2019, 27 (12), 3709, https://doi.org/10.1016/j.celrep.2019.05.077.
[14] R. Sklavenitis-Pistofidis, M. P. Aranha, R. A. Redd, J. Baginska, N. J. Haradhvala, M. Hallisey, A. K. Dutta, A. Savell, S. Varmeh, D. Heilpern-Mallory, S. Ujwary, O. Zavidij, F. Aguet, N. K. Su, E. D. Lightbody, M. Bustoros, S. Tahri, T. H. Mouhieddine, T. Wu, L. Flechon, S. Anand, J. M. Rosenblatt, J. Zonder, J. J. Vredenburgh, A. Boruchov, M. Bhutani, S. Z. Usmani, J. Matous, A. J. Yee, A. Jakubowiak, J. Laubach, S. Manier, O. Nadeem, P. Richardson, A. Z. Badros, M. V. Mateos, L. Trippa, G. Getz, I. M. Ghobrial, Cancer Cell 2022, 40 (11), 1358, https://doi.org/10.1016/j.ccell.2022.10.017.
[15] F. Tang, C. Barbacioru, Y. Wang, E. Nordman, C. Lee, N. Xu, X. Wang, J. Bodeau, B. B. Tuch, A. Siddiqui, K. Lao, M. A. Surani, Nat Methods 2009, 6 (5), 377, https://doi.org/10.1038/nmeth.1315.
[16] Z. Li, M. Wang, J. Tan, L. Zhu, P. Zeng, X. Chen, L. Xie, R. Duan, B. Chen, T. Tao, R. Wang, X. Wang, W. Su, Cell Rep Med 2022, 3 (8), 100699, https://doi.org/10.1016/j.xcrm.2022.100699.
[17] X. Ma, J. Guo, K. Liu, L. Chen, D. Liu, S. Dong, J. Xia, Q. Long, Y. Yue, P. Zhao, F. Hu, Z. Xiao, X. Pan, K. Xiao, Z. Cheng, Z. Ke, Z. S. Chen, C. Zou, Mol Cancer 2020, 19 (1), 147, https://doi.org/10.1186/s12943-020-01264-9.
[18] a) R. Li, J. Liu, P. Yi, X. Yang, J. Chen, C. Zhao, X. Liao, X. Wang, Z. Xu, H. Lu, H. Li, Z. Zhang, X. Liu, J. Xiang, K. Hu, H. Qi, J. Yu, P. Yang, S. Hou, Adv Sci (Weinh) 2023, e2206623, https://doi.org/10.1002/advs.202206623; b) C. Finkbeiner, I. Ortuno-Lizaran, A. Sridhar, M. Hooper, S. Petter, T. A. Reh, Cell Rep 2022, 38 (4), 110294, ttps://doi.org/10.1016/j.celrep.2021.110294.
[19] a) A. P. Voigt, E. Binkley, M. J. Flamme-Wiese, S. Zeng, A. P. DeLuca, T. E. Scheetz, B. A. Tucker, R. F. Mullins, E. M. Stone, Cells 2020, 9 (2), https://doi.org/10.3390/cells9020438; b) A. P. Voigt, K. Mulfaul, N. K. Mullin, M. J. Flamme-Wiese, J. C. Giacalone, E. M. Stone, B. A. Tucker, T. E. Scheetz, R. F. Mullins, Proc Natl Acad Sci USA 2019, 116 (48), 24100, https://doi.org/10.1073/pnas. 1914143116; c) J. Collin, R. Queen, D. Zerti, D. H. Steel, C. Bowen, M. Parulekar, M. Lako, Invest Ophthalmol Vis Sci 2021, 62 (6), 18, https://doi.org/10.1167/iovs.62.6.18.
[20] J. Choi, J. Li, S. Ferdous, Q. Liang, J. R. Moffitt, R. Chen, Nat Commun 2023, 14 (1), 4929, https://doi.org/10.1038/s41467-023-40674-3
[21] B. Dorgau, J. Collin, A. Rozanska, D. Zerti, A. Unsworth, M. Crosier, R. Hussain, J. Coxhead, T. Dhanaseelan, A. Patel, J. C. Sowden, D. R. FitzPatrick, R. Queen, M. Lako, Nat Commun 2024, 15 (1), 3567, https://doi.org/10.1038/s41467-024-47933-x.
[22] M. Boyce, K. F. Bryant, C. Jousse, K. Long, H. P. Harding, D. Scheuner, R. J. Kaufman, D. Ma, D. M. Coen, D. Ron, J. Yuan, Science 2005, 307 (5711), 935, https://doi.org/10.1126/science.1101902.
[23] M. Matsuoka, Y. Komoike, Int J Mol Sci 2015, 16 (7), 16275, ttps://doi.org/10.3390/ijms160716275.
[24] a) N. Z. Gregori, B. L. Lam, G. Gregori, S. Ranganathan, E. M. Stone, A. Morante, F. Abukhalil, P. R. Aroucha, Ophthalmology 2013, 120 (1), 169, https://doi.org/10.1016/j.ophtha.2012.07.051; b) C. Vijayasarathy, S. P. B. Sardar Pasha, P. A. Sieving, Prog Retin Eye Res 2022, 87, 100999, https://doi.org/10.1016/j.preteyeres.2021.100999.
[25] L. L. Molday, W. W. Wu, R. S. Molday, J Biol Chem 2007, 282 (45), 32792, https://doi.org/10.1074/jbc. M706321200.
[26] a) S. S. Nikonov, A. Lyubarsky, M. E. Fina, E. S. Nikonova, A. Sengupta, C. Chinniah, X. Q. Ding, R. G. Smith, E. N. Pugh, Jr., N. Vardi, A. Dhingra, J Neurosci 2013, 33 (12), 5182, https://doi.org/10.1523/JNEUROSCI.5204-12.2013; b) G. Arno, G. E. Holder, C. Chakarova, S. Kohl, N. Pontikos, A. Fiorentino, V. Plagnol, M. E. Cheetham, A. J. Hardcastle, A. R. Webster, M. Michaelides, U. K. I. R. D. Consortium, JAMA Ophthalmol 2016, 134 (8), 924, https://doi.org/10.1001/jamaophthalmol.2016.1543.
[27] a) V. P. Sarthy, H. Sawkar, V. J. Dudley, Curr Eye Res 2015, 40 (11), 1181, https://doi.org/10.3109/02713683.2014.982828; b) S. Kassumeh, S. Leopold, R. Fuchshofer, C. N. Thomas, S. G. Priglinger, E. R. Tamm, A. Ohlmann, Cells 2020, 9 (2), https://doi.org/10.3390/cells9020277; c) Y. Kobayashi, S. Watanabe, A. L. C. Ong, M. Shirai, C. Yamashiro, T. Ogata, F. Higashijima, T. Yoshimoto, T. Hayano, Y. Asai, N. Sasai, K. Kimura, Dis Model Mech 2021, 14 (11), https://doi.org/10.1242/dmm.048962.
[28] M. Haeri, B. E. Knox, J Ophthalmic Vis Res 2012, 7 (1), 45.
[29] R. C. Wek, Cold Spring Harb Perspect Biol 2018, 10 (7), https://doi.org/10.1101/cshperspect.a032870
[30] M. C. Bell, S. E. Meier, A. L. Ingram, J. F. Abisambra, Curr Alzheimer Res 2016, 13 (2), 150, https://doi.org/10.2174/1567205013666151218145431
[31] Y. Inokuchi, Y. Nakajima, M. Shimazawa, T. Kurita, M. Kubo, A. Saito, H. Sajiki, T. Kudo, M. Aihara, K. Imaizumi, M. Araie, H. Hara, Invest Ophthalmol Vis Sci 2009, 50 (1), 334, https://doi.org/10.1167/iovs.08-2123.
[32] S. Park, Y. Lim, D. Lee, R. Elvira, J. M. Lee, M. R. Lee, J. Han, Cells 2018, 7 (12), https://doi.org/10.3390/cells7120254.
[33] a) Y. Komoike, H. Inamura, M. Matsuoka, Arch Toxicol 2012, 86 (1), 37, https://doi.org/10.1007/s00204-011-0742-x; b) W. Yang, E. Tiffany-Castiglioni, H. C. Koh, I. H. Son, Toxicol Lett 2009, 191 (2-3), 203, https://doi.org/10.1016/j.toxlet.2009.08.024; c) S. I. Lee, K. L. Kang, S. I. Shin, Y. Herr, Y. M. Lee, E. C. Kim, J Periodontal Res 2012, 47 (3), 299, https://doi.org/10.1111/j.1600-0765.2011.01432.x.
[34] B. A. Scruggs, S. Bhattarai, M. Helms, I. Cherascu, A. Salesevic, E. Stalter, J. Laird, S. A. Baker, A. V. Drack, PLoS One 2022, 17 (12), e0276298, https://doi.org/10.1371/journal.pone.0276298.
[35] a) S. H. Min, L. L. Molday, M. W. Seeliger, A. Dinculescu, A. M. Timmers, A. Janssen, F. Tonagel, N. Tanimoto, B. H. Weber, R. S. Molday, W. W. Hauswirth, Mol Ther 2005, 12 (4), 644, https://doi.org/10.1016/j.ymthe.2005.06.002; b) A. Janssen, S. H. Min, L. L. Molday, N. Tanimoto, M. W. Seeliger, W. W. Hauswirth, R. S. Molday, B. H. Weber, Mol Ther 2008, 16 (6), 1010, https://doi.org/10.1038/mt.2008.57; c) L. C. Byrne, B. E. Ozturk, T. Lee, C. Fortuny, M. Visel, D. Dalkara, D. V. Schaffer, J. G. Flannery, Gene Ther 2014, 21 (6), 585, https://doi.org/10.1038/gt.2014.31.
[36] M. M. Oliveira, E. Klann, Semin Cell Dev Biol 2022, 125, 101, https://doi.org/10.1016/j.semcdb.2021.07.009.
[37] A. Salminen, A. Kauppinen, J. M. Hyttinen, E. Toropainen, K. Kaarniranta, Mol Med 2010, 16 (11-12), 535, https://doi.org/10.2119/molmed.2010.00070.
[38] a) G. Dong, Y. Liu, L. Zhang, S. Huang, H. F. Ding, Z. Dong, Am J Physiol Renal Physiol 2015, 308 (3), F267, https://doi.org/10.1152/ajprenal.00629.2014; b) B. J. Guan, D. Krokowski, M. Majumder, C. L. Schmotzer, S. R. Kimball, W. C. Merrick, A. E. Koromilas, M. Hatzoglou, J Biol Chem 2014, 289 (18), 12593, https://doi.org/10.1074/jbc.M113.543215; c) I. A. Nikonorova, E. T. Mirek, C. C. Signore, M. P. Goudie, R. C. Wek, T. G. Anthony, J Biol Chem 2018, 293 (14), 5005, https://doi.org/10.1074/jbc.RA117.001625; d) L. S. D'Abronzo, S. Bose, M. E. Crapuchettes, R. E. Beggs, R. L. Vinall, C. G. Tepper, S. Siddiqui, M. Mudryj, F. U. Melgoza, B. P. Durbin-Johnson, R. W. deVere White, P. M. Ghosh, Oncogene 2017, 36 (46), 6359, https://doi.org/10.1038/one.2017.233; e) S. Y. Sun, L. M. Rosenberg, X. Wang, Z. Zhou, P. Yue, H. Fu, F. R. Khuri, Cancer Res 2005, 65 (16), 7052, https://doi.org/10.1158/0008-5472.CAN-05-0917; f) A. C. Hsieh, M. Costa, O. Zollo, C. Davis, M. E. Feldman, J. R. Testa, O. Meyuhas, K. M.

1. A multiomics approach, which integrate single-cell RNA-sequencing (scRNA-seq) and spatiotemporal transcriptomics (ST) offering potential for dissecting transcriptional networks and revealing cell-cell interactions involved in biomolecular pathomechanisms.

2. A multimodal system, comprising high-throughput scRNA-seq and ST to elucidate XLRS-specific transcriptomic signatures.

3. A disease model, which is a genetically engineered mice Rs1emR209C.

4. A disease model, which comprises a patient-derived retinal organoids harboring the patient-specific p.R209C mutation.

5. The disease model of claim 3, which is a X-linked retinoschisis (XLRS)-like model.

6. The disease model of claim 3, wherein the endoplasmic reticulum (ER) stress/eIF2 signaling, mTOR pathway, and the regulation of eIF4 and p70S6K pathways as chronically enriched and highly conserved disease pathways.

7. A method for treating retinal disease in a subject, which comprising administering to the subject an AAV mediated RS1 gene delivery together with a therapeutic agent targeting the endoplasmic reticulum (ER) stress/eIF2 signaling, mTOR pathway, and the regulation of eIF4 and p70S6K pathways, which ultimately improves bipolar cell integrity, postsynaptic transmission, disorganized retinal architecture and electrophysiological responses.

8. The method of claim 6, wherein the retinal disease is X-linked retinoschisis (XLRS).
